(19) United States
(12) Patent Application Publication
(10) Pub. No.: US 2023/0250439 A1
(43) Pub. Date: Aug. 10, 2023

(54) POLYNUCLEOTIDE SECONDARY STRUCTURE

(71) Applicant: ModernaTX, Inc., Cambridge, MA (US)

(72) Inventors: David Mauger, Arlington, MA (US); Iain Mcfadyen, Arlington, MA (US); Vladimir Presnyak, Manchester, NH (US)

(73) Assignee: ModernaTX, Inc., Cambridge, MA (US)

(21) Appl. No.: 18/093,119

(22) Filed: Jan. 4, 2023

Related U.S. Application Data
(62) Division of application No. 16/483,012, filed on Aug. 1, 2019, now abandoned.
(60) Provisional application No. 62/453,482, filed on Feb. 1, 2017.

(51) Int. Cl. C12N 15/67; A61K 48/00

(52) U.S. Cl. CPC C12N 15/67; A61K 48/0066

(57) ABSTRACT

The disclosure relates to synthetic thermostable polynucleotides, as well as methods of synthesizing and delivering the polynucleotides.

### RELATED APPLICATION

This application claims the benefit under 35 U.S.C. § 119(e) of U.S. provisional application No. 62/453,482, filed Feb. 1, 2017, which is incorporated by reference herein in its entirety.

### BACKGROUND

It is of great interest in the fields of therapeutics, diagnostics, reagents and for biological assays to be able to design, synthesize and deliver a nucleic acid, e.g., a ribonucleic acid (RNA) for example, a messenger RNA (mRNA) inside a cell, whether in vitro, in vivo, in situ or ex vivo, such as to effect physiologic outcomes which are beneficial to the cell, tissue or organ and ultimately to an organism. One beneficial outcome is to cause intracellular translation of the nucleic acid and production of at least one encoded peptide or polypeptide of interest. In some cases, RNA is synthesized in the laboratory in order to achieve these methods.

### SUMMARY OF INVENTION

The invention involves, at least in part, the discovery of position-dependent structure profiles that result in high rates of protein expression. Provided herein are synthetic structurally stable RNA (e.g., messenger RNA (mRNA)) with nucleotide chemistries and primary sequences which may be used to enhance protein translation.

The efficacy of mRNA therapeutics critically depends on evasion of the innate immune system and ability to robustly translate a therapeutic protein from exogenously introduced mRNA. Chemical modification of the RNA has historically been used to evade nucleic acid sensors; however, there are conflicting reports as to the levels of protein that ensue from translation of modified mRNAs. Through comprehensive functional analysis, the present disclosure demonstrates that the rules by which primary RNA sequence determine level of protein expression are not uniform across all nucleotide chemistries, and that protein expression is the result of both RNA sequence and nucleotide chemistry. Further, it was found that modification of nucleotide chemistry grossly alters both the global thermodynamic profile and the discrete structural conformation of the RNA. Further, nucleotide chemistries with intrinsic high thermodynamic stability are less sensitive to primary sequence variation and moreover for those chemistries with weak thermodynamic stability; high-expressing sequences are stabilized relative to other poorly-expressing variants. Regardless of nucleotide chemistry, high-expressing sequences contain a uniform, position-dependent structure profile defined by a flexible leader region and a high degree of structural stability throughout the remainder of the molecule. The functional correlation to this structure profile was found to be greatest for those chemistries with weak intrinsic thermodynamic stability and great sensitivity to primary sequence variation. When evaluating the mechanism by which structured mRNAs occupy a privileged expression state, structured mRNAs do not persist in the cell any longer than their unstructured counterparts, but rather associate with a greater number of ribosomes; indicating the advantage is in the translation, not stability, of a given mRNA. In sum, the present disclosure provides critical insight into important structural features which yield high therapeutically relevant levels of protein in vivo, and further presents a comprehensive model inform on the translatability of exogenously introduced mRNAs. Thus, the invention in some aspects includes high expressing mRNA useful in therapeutic indications.

The present disclosure, in some aspects, includes a synthetic thermostable mRNA comprising: a nucleic acid, ie ribonucleic acid, having a primary sequence and including at least a portion of an open reading frame (ORF), wherein each nucleotide of the nucleic acid has a defined chemistry, wherein the primary sequence and the chemistry of the nucleotides contribute to a thermostable mRNA structure having a mRNA minimum free energy (MFE) value; and wherein the mRNA MFE value is less than a median distribution MFE value of synonymous variants. The term including, also sometimes referred to as encoding, in this context means comprising.

In some embodiments, at least one nucleotide is a chemically modified nucleotide. In other embodiments, at least 50% of uracil in the nucleic acid have a chemical modification. In an embodiment, the chemical modification is N1-methyl-pseudouridine. In some embodiments, the chemical modification is pseudouridine. In some embodiments, the chemical modification is 5-methoxy-uridine.

In some embodiments, the mRNA MFE is within a top 0.1% of low MFE as defined computationally of synonymous variants.

In some embodiments, the thermostable mRNA has secondary structure capability and wherein greater than 50% of the thermostable mRNA forms secondary structure at 37° C. as defined by UV-melting analysis. In other embodiments, the thermostable mRNA has secondary structure capability and greater than 70% of the thermostable mRNA forms secondary structure at 37° C. as defined by UV-melting analysis. In another embodiment, the thermostable mRNA has secondary structure capability and greater than 90% of the thermostable mRNA forms secondary structure at 37° C. as defined by UV-melting analysis.

In some embodiments, the thermostable mRNA has a SHAPE reactivity of less than 0.8.

In some embodiments, the nucleic acid encodes the entire ORF. In some embodiments, the nucleic acid encodes the entire ORF except for the first 30 nucleotides of the ORF. In another embodiment, the nucleic acid encodes the entire ORF except for the first 60 nucleotides of the ORF.

In some embodiments, the nucleic acid further comprises a 3′ untranslated region (UTR).

In other embodiments, the nucleic acid further comprises a 5′ flexible region that comprises a 5′UTR. In an embodiment, the flexible region comprises the first 30 nucleotides of the ORF linked to the 3′ end of the 5′UTR. In some embodiments, the flexible region comprises the first 60 nucleotides of the ORF linked to the 3′ end of the 5′UTR. In other embodiments, less than 30% of the flexible region forms secondary structure at 37° C. as defined by UV-melting analysis. In some embodiments, less than 20% of the flexible region forms secondary structure at 37° C. as defined by UV-melting analysis. In another embodiment, less than 10% of the flexible region forms secondary structure at 37° C. as defined by UV-melting analysis. In some embodiments, the flexible region has a SHAPE reactivity of greater than 1.5.

In some embodiments, the primary sequence of the nucleic acid has a low U content, wherein less than 24% of the nucleotides are U.

In some embodiments, the mRNA is formulated within a lipid nanoparticle.

In other embodiments, the MFE values are normalized for 1,000 nucleotide sequences.

The disclosure, in other aspects, provides a method for producing highly expressing mRNA, the method comprising determining a flexibility value for each nucleotide within a population of synonymous RNA, determining a SHAPE reactivity for each RNA corresponding to the primary sequence and chemistry of the nucleotides based on the combined flexibility values of the nucleotides, selecting a RNA from the population having a SHAPE reactivity of less than 1.0, and synthesizing highly expressing mRNA based on the primary sequence and chemistry of the nucleotides of the selected RNA having a SHAPE reactivity of less than 1.0.

In some embodiments, the highly expressing mRNA is determined to be highly expressing relative to a corresponding wild type chemically unmodified RNA and the highly expressing mRNA produces more protein than the wild type RNA. In other embodiments, the highly expressing mRNA produces at least 10% more protein than the wild type RNA.

In another embodiment, the highly expressing mRNA has a SHAPE reactivity of less than 0.8.

In some embodiments, the primary sequence of the RNA has a low U content, wherein less than 24% of the nucleotides are U. In other embodiments, the primary sequence of the RNA is thermodynamically stable. In some embodiments, at least some of the nucleotides have a 5-methoxy-uridine chemical modification. In other embodiments, the primary sequence of the RNA is thermodynamically unstable. In some embodiments, at least some of the nucleotides have a N1-methyl-pseudouridine or pseudouridine chemical modification.

In some embodiments, the highly expressing mRNA has an mRNA minimum free energy (MFE) value within a top 0.1% of low MFE as defined computationally of synonymous variants. In other embodiments, the highly expressing mRNA has secondary structure capability and wherein greater than 50% of the mRNA forms secondary structure at 37° C. as defined by UV-melting analysis. In further embodiments, the highly expressing mRNA has secondary structure capability and wherein greater than 70% of the thermostable mRNA forms secondary structure at 37° C. as defined by UV-melting analysis. In some embodiments, the highly expressing mRNA has secondary structure capability and wherein greater than 90% of the thermostable mRNA forms secondary structure at 37° C. as defined by UV-melting analysis.

Another aspect of the present disclosure includes a thermostable mRNA comprising a flexible region comprising a first set of nucleotides having a primary sequence and including a 5′ untranslated region (UTR), wherein the first set of nucleotides including the 5′ UTR have a first flexibility value based on folding conformation propensity of the primary sequence and thermodynamic stability of nucleotide chemistry; and a thermostable region comprising a second set of nucleotides having a primary sequence and including at least a portion of an open reading frame (ORF) and a 3′ UTR, wherein the second set of nucleotides including the ORF and 3′ UTR have a second flexibility value; wherein the flexible region is linked 5′ to the thermostable region and wherein the first flexibility value is greater than the second flexibility value, indicating that the flexible region has greater flexibility than the thermostable region.

In some embodiments, the mRNA comprises at least one chemical modification. In another embodiment, at least 50% of uracil in the open reading frame have a chemical modification. In other embodiments, the chemical modification is N1-methyl-pseudouridine. In some embodiments, at least 30% of the N1-methyl-pseudouridine modifications are in the first set of nucleotides. In other embodiments, at least 30% of the N1-methyl-pseudouridine modifications are in the second set of nucleotides. In some embodiments, the chemical modification is pseudouridine. In another embodiment, at least 30% of the pseudouridine modifications are in the first set of nucleotides. In some embodiments, at least 30% of the pseudouridine modifications are in the second set of nucleotides. In another embodiment, the chemical modification is 5-methoxy-uridine. In some embodiments, at least 30% of the 5-methoxy-uridine modifications are in the first set of nucleotides. In another embodiment, at least 30% of the 5-methoxy-uridine modifications are in the second set of nucleotides.

In some embodiments, the first set of nucleotides encodes a first segment of the ORF immediately following the 5′ UTR. In another embodiment, the first segment of the ORF comprises a first 10 codons of the ORF. In other embodiments, the first segment of the ORF comprises a first 30 codons of the ORF. In some embodiments, the second set of nucleotides encodes an entire ORF.

In some embodiments, the flexible region has SHAPE reactivity value of greater than 1.5. In other embodiments, the thermostable region has SHAPE reactivity value of less than 0.8. In some embodiments, the first flexibility value is 2-10 times greater than the second flexibility value. In other embodiments, the first flexibility value is 10-70% greater than the second flexibility value. In some embodiments, 0-20% of the first set of nucleotides have a high thermodynamic stability. In another embodiment, at least 30% of the second set of nucleotides have a high thermodynamic stability.

In other embodiments, the mRNA is formulated within a lipid nanoparticle.

Another aspect of the present disclosure includes a method of synthesizing a thermostable mRNA, the method comprising binding a first polynucleotide comprising a flexible region comprising a first set of nucleotides having a primary sequence and including a 5′ untranslated region (UTR), wherein the first set of nucleotides including the 5′ UTR have a first flexibility value based on folding conformation propensity of the primary sequence and thermodynamic stability of nucleotide chemistry, wherein the first polynucleotide is conjugated to a solid support, and a second polynucleotide comprising a thermostable region comprising a second set of nucleotides having a primary sequence and including at least a portion of an open reading frame (ORF), wherein the second set of nucleotides including the ORF have a second flexibility value; ligating the 3′-terminus of the first polynucleotide to the 5′-terminus of the second polynucleotide under suitable conditions, wherein the suitable conditions comprise a DNA Ligase, thereby producing a first ligation product; ligating the 5′ terminus of a third polynucleotide comprising a 3′-UTR to the 3′-terminus of the first ligation product under suitable conditions, wherein the suitable conditions comprise an RNA Ligase, thereby producing a second ligation product; and releasing the second ligation product from the solid support, thereby producing the thermostable mRNA.

An additional aspect of the present disclosure includes a thermostable mRNA comprising an mRNA having an open reading frame including a polypeptide and a pharmaceutically acceptable carrier or excipient, wherein the mRNA is preparable by ligating a flexible region of RNA comprising a first set of nucleotides having a primary sequence and including a 5′ untranslated region (UTR) to a second polynucleotide comprising a thermostable region comprising a second set of nucleotides having a primary sequence and including at least a portion of an open reading frame (ORF) and a 3′ UTR.

The present disclosure, in another aspect, provides a method of delivering a peptide to a subject, comprising administering to a subject a thermostable mRNA, wherein the thermostable mRNA comprises a flexible region having a first flexibility value based on folding conformation propensity of the primary sequence and thermodynamic stability of nucleotide chemistry; and a thermostable region having a second flexibility value; wherein the flexible region is linked 5′ to the thermostable region and wherein the first flexibility value is greater than the second flexibility value, indicating that the flexible region has greater flexibility than the thermostable region, and wherein the mRNA produces a detectable amount of peptide in a tissue of the subject.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF THE DRAWINGS

The foregoing and other objects, features and advantages will be apparent from the following description of particular embodiments of the invention, as illustrated in the accompanying drawings in which like reference characters refer to the same parts throughout the different views. The drawings are not necessarily to scale, emphasis instead being placed upon illustrating the principles of various embodiments of the invention.

FIGS. 1A-1E show the inclusion of modified nucleotides in mRNA alters protein expression. FIG. 1A shows the chemical structures of uridine and four modified nucleosides: pseudouridine (Ψ). N′-methyl-pseudouridine (m¹Ψ), 5-methyoxy-uridine (mo⁵U), and 5-methyl-cytidine (m⁵C). FIG. 1B is a schematic of the human erythropoietin (hEpo) mRNA sequence variants. The coding sequence (wide grey boxes) is flanked by 5′ and 3′ untranslated regions (UTRs, narrow white boxes) and a 3′ 100-nucleotide poly-A tail. Eight hEpo sequences combined one of two “head” regions (dark grey box, H_{A} and He) including the first 30 amino acids (90 nucleotides) and one of four “body” regions (light grey box. E₁ through E₄) encoding the remainder of the hEpo CDS. FIG. 1C is a graph depicting eGFP expression in HeLa cells, showing that the primary sequence of the mRNA impacts the relative potency of different mRNAs. Fluorescence intensity of HeLa cells following transfection with lipofectamine alone (−) or four different eGFP sequence variants (G₁-G₄) containing uridine, m¹Ψ, Ψ, m⁵C/Ψ, or mo⁵U is shown. The mean and range of expression for each modification is shown below the graph. FIG. 1D shows an analysis of eight different synonymous hEPO variants (described in FIG. 1B, above) using N1-methyl-pseudouridine, unmodified uracil, and 5-methoxy-uridine in HeLa cells and primary hepatocytes. Levels of secreted hEpo protein measured by ELISA in ng/mL following transfection plus one “codon optimized” (E_{CO}) variant containing uridine, m¹Ψ, or mo⁵U are shown. FIG. 1E shows the serum concentrations of hEpo protein measured by ELISA in BALB-c mice (five per group) following IV injection of LNP-formulated mRNA of 6 sequence variants (described in FIG. 1B, above) plus one “codon optimized” variant (E_{CO}) (Welch et al., 2009a) containing m¹Ψ or mo⁵U. Individual animals (dots) with mean and standard error (black lines). The mean and range of expression for each modification are shown below the graph.

FIGS. 2A-2C show an exploration of two different RNA chemistries (1mψ and 5moU) across as set 42 synonymous sequence variants of firefly luciferase. FIG. 2A is a graph showing normalized luciferase activity in HeLa cells with the two different chemistries. FIG. 2B shows the production of luciferase protein in vivo measured 6 hours, post-injection, through the whole animal. The liver was found to be the main site of protein expression. FIG. 2C shows 1mψ luciferase expression in CD-1 cells (left) and 5moU luciferase expression in CD-1 cells (right).

FIGS. 3A-3B show that modified nucleotides induce global structural changes in mRNA. FIG. 3A shows the optical melting profiles of Luc sequence variants L₁₈, L₁₅, and L₃₂ containing uridine (unmodified), m¹Ψ, or mo⁵U showing the change in UV absorbance at 260 nm (y-axis) as a function of temperature (x-axis). FIG. 3B shows nearest neighbor thermodynamic parameters for Watson-crick base pairs (x-axis) containing uridine (circles, values from (Xia et al., 1998)), Ψ (diamonds), m¹Ψ (squares), or mo⁵U (triangles). The position of modified nucleotides for each nearest neighbor is highlighted in red. Parameters were derived by linear regression to UV-melting data from X short oligonucleotides containing global substitutions, as described in (Xia et al., 1998).

FIGS. 4A-4C illustrate that SHAPE data reveal a bipartite relationship between mRNA structure and protein expression. FIG. 4A shows median SHAPE reactivity values (33-nt sliding window) for hEpo sequence variants E_{CO} (top) and H_{A}E₃ (bottom) containing m¹Ψ (left) or mo⁵U (right) shown as a heatmap: highly reactive, moderately reactive (grey), and lowly reactive. hEpo serum concentrations observed in mice upon injection of LNP-formulated mRNA are shown to the right, taken from FIG. 1E. The 5′ and 3′ UTRs (thin white boxes), H_{A} coding sequence (dark grey box). E₂ coding sequence (light grey box), and poly A tail are shown in the schematics below. FIG. 4B shows structure-function relationships. Pearson correlations between median windowed SHAPE reactivity value and expression in HeLa cells (y-axis), taken from FIG. 44A plotted for windows centered at indicated nucleotide position (x-axis) for Luc sequence variants containing m¹Ψ (16 variants) or mo⁵U (12 variants). Insets, example scatterplots of SHAPE reactivity values (x-axis) versus expression (RLU, y-axis) for windows centered at position 24 (left) and 979 (right) for m¹Ψ-containing mRNAs, with linear regressions and Pearson correlations. FIG. 4C shows the same parameters as in FIG. 4A, but for firefly Luc sequence variants L₁₈, L₈, and L₃₂. Total luminescence values are also shown, taken from FIGS. 44E and 44F.

FIGS. 5A-5D show the kinetics of protein expression and mRNA degradation in AML12 cells. FIG. 5A shows luciferase expression over time in transfected AML21 liver cells using two different chemistries. FIG. 5B shows the correlation between the average rate of protein production over the first 7 hours post-transfection in AML12 cells (y-axis) and in vivo Luc expression 6 hours post-injection (x-axis) for 11 firefly Luc sequence variants containing m¹Ψ (left) or mo⁵U (right), with linear regression line and Pearson correlations. FIG. 5C shows a time course (1 to 7 hours post-transfection, x-axis) of expression (luminescence, RLU, y-axis) for 11 Luc sequence variants containing m¹Ψ (left) or mo⁵U (right) in AML12 cells. FIG. 5D shows the levels of mRNA remaining (y-axis) in AML12 cells over time in hours (x-axis) following electroporation of mRNA variants containing either m¹Ψ (left chart) or mo⁵U (right chart). RNA levels as measured by bDNA assay are shown for three Luc constructs displaying a range of expression phenotypes (L₁₈, L₇, L₂₄) and a negative control lacking the polyA tail (Tailless) that is subject to rapid degradation, with exponential decay trend lines.

FIG. 6 illustrates that traditional metrics of primary sequence are poor predictors of chemistry-specific expression.

FIG. 7 shows that biochemical data (SHAPE reactivity scores) can reveal a structure-function relationship between mRNA and protein expression.

FIG. 8 shows that structure-function relationships are dependent on the position within the RNA.

FIG. 9 is two graphs providing confirmation of the expression pattern of luciferase sequences across production batches and processes. Significant process changes (alpha v. equimolar, RP-HPLC) were introduced between synthesis dates.

FIG. 10 shows that in vitro assays are moderately predictive of expression in vivo.

FIG. 11 shows that sequences that display different chemistry-dependent expression differ in their UV melting profiles.

FIG. 12 shows that high-expressing mo⁵U sequences adopt a physical profile more similar to m¹Ψ.

FIG. 13 shows that high- and low-expressing sequences of uniform chemistry can be differentiated by their melting profiles.

FIG. 14 shows that the structure-function relationships are consistent across reporter proteins (m¹Ψ hEPO).

FIG. 15 shows that the structure-function relationships are consistent across reporter proteins (mo⁵U hEPO).

FIG. 16 is a schematic depicting the “thumb” model.

FIG. 17 shows the thermodynamic landscape for modified nucleotides, as demonstrated by AU nearest-neighbor parameters for uracil derivatives.

FIG. 18 shows that the distribution of MFEs for random hEPO sequences space shift as a function of nucleotide chemistry.

FIG. 19 shows the propensity for generating high-expressing mRNA sequences can be explained by distribution shift.

FIGS. 20A-20C show that the structure near the start codon impacts expression of m¹Ψ. FIG. 20A is a schematic of 3 original Luc variants (left, L₇, L₁₈, and L₂₇) and 2 chimeric constructs (right, L₁₈A-L₂₇B and L₁₈A-L₇B) which combine regions near the start codon (designated ‘A’) and remainder of CDS (designated ‘B’). FIG. 20B shows the expression in primary mouse hepatocytes (RLU, x-axis) for 2 original Luc variants (L₇ and L₂₇) and 2 chimeric constructs (y-axis) containing m¹Ψ. FIG. 20C shows median SHAPE reactivity values (y-axis, 33-nt sliding window) for Luc sequence (L₁₈A-L₂₇B and L₂₇ top. L₁₈A-L₇B and L₇ bottom) containing m¹Ψ for the 60-nucleotide region (x-axis) within ‘A’ centered around the start codon (indicated by lower rectangle).

FIG. 21 is a schematic depicting massively-parallel screening of open reading frame variants.

FIG. 22 is a schematic depicting Selective 2′-Hydroxyl Acylation analyzed by Primer Extension (SHAPE) and the process for probing RNA structure flexibility.

FIG. 23 depicts chemistry-sensitive sequence variants.

FIG. 24 shows an in vivo validation of the structure-based design scheme.

FIG. 25 shows dosing studies for the in vivo validation of the structure-based design scheme.

FIG. 26 demonstrates that sequences that express well in each chemistry have similar UV melting profiles.

FIG. 27 demonstrates that sequences that express poorly in each chemistry have similar UV melting profiles.

FIG. 28 shows that, with respect to mo⁵U chemistry, high-expressing sequences are more thermostable than their lower-expressing counterparts.

FIG. 29 shows the total folding energy of luciferase variants with different chemistries. Similar to hEPO, high-expressing variants (m¹Ψ chemistry) occupy the most structured portion of the MFE space.

FIG. 30 demonstrates that high-expressing luciferase variants have low MFE independent of GC content.

FIG. 31 shows that GC and MFE correlated for both m¹Ψ and mo⁵U chemistries.

FIG. 32 shows the expression of luciferase variants cannot be explained by the selection of codons with modified nucleotides.

FIG. 33 shows that the selection of the most frequently used codons does not drive luciferase expression, as evidenced by serine.

FIG. 34 demonstrates that deterministic codon selection has an inconsistent impact on protein expression.

FIG. 35 shows expression and activity data from engineered sequences (ELP-01). Mouse hepatocytes were transfected with mRNAs through electroporation and assayed at 24 hours.

FIG. 36 shows expression and activity data from designs specific to mo⁵U (ELP-01).

FIG. 37 shows that, with respect to m¹Ψ chemistry, high-expressing sequences are more thermostable than their low-expressing counterparts.

FIGS. 38A-38G show SHAPE structure probing, revealing widespread conformation changes induced by m¹Ψ or moU substitution of uridine. FIG. 38A is a schematic of SHAPE-MaP methodology. The SHAPE reagent 1M6 reacts with the 2′ hydroxyl position of flexible nucleotides, creating a bulky covalent adduct which results in increased mutation rates in the cDNA read-out by NGS. FIG. 38B shows mutation rates for untreated (light grey, −) and treated (dark grey. +) samples for hEpo sequence variant H_{A}E3 containing uridine, m¹Ψ or mo⁵U, as indicated below the graph. FIG. 38C shows SHAPE reactivity per nucleotide (y-axis) for hEpo sequence variant H_{A}E₃ containing m¹Ψ: highly reactive, moderately reactive, or lowly reactive. Nucleotides with insufficient NGS data are indicated with grey lines under the x-axis. The 5′ and 3′ UTRs (thin white boxes). H_{A} coding sequence (dark grey box), E3 coding sequence (light grey box), poly-A tail, and the position of nucleotides in subfigure D (518-595) are shown in the schematic below. FIG. 38D shows median SHAPE reactivity values (33-nt sliding window) for hEpo sequence variant H_{A}E₂ containing uridine (top), m¹Ψ (middle), or mo⁵U (bottom) shown as a heatmap: highly reactive, moderately reactive (grey), and lowly reactive. The 5′ and 3′ UTRs (thin white boxes), H_{A} coding sequence (dark grey box). E₃ coding sequence (light grey box), and poly A tail are shown in the schematic above. FIG. 38E shows SHAPE reactivities for a region of hEpo sequence variant H_{A}E₃ that undergoes modification induced structural rearrangement (nucleotides 518-595) for mRNAs containing uridine, m¹Ψ, or mo⁵U. FIG. 38F is a diagram of SHAPE-directed minimum free energy secondary structure for hEpo sequence variant H_{A}E₃ containing uridine, m¹Ψ, or mo⁵U. Location of the 5′ end of the mRNA is indicated. FIG. 38G illustrates the distribution of common and unique base pairs between the SHAPE-directed minimum free energy predictions for hEpo sequence variant H_{A}E₃ containing uridine, m¹Ψ, or mo⁵U, which is shown as a Venn diagram.

FIGS. 39A-39E show that the ribosomal association of modified mRNAs drive expression differences. FIGS. 39A-39B show individual gradient sedimentation profiles as heat maps for 10 Luc sequence variants (vertical axis) containing m¹Ψ (FIG. 39A) or mo⁵U (FIG. 39B). Darker shades indicate higher relative concentration of mRNA in the gradient fraction indicated. Gradient fractions were monitored by UV absorbance (260 nm) (black line) to identify fractions containing free RNA, monosomes, and polysomes. FIGS. 39C and 39D show average gradient sedimentation profiles for 11 Luc sequence variants containing m¹Ψ (FIG. 39C) or mo⁵U (FIG. 39D). Gradient fractions were monitored by UV absorbance (260 nm) (black line) to identify fractions containing free RNA, monosomes, and polysomes (indicated below the plot). FIG. 39E shows the correlation between the percentage of mRNA associated with ribosomes (monosomes and polysomes fractions in AML12 cells (x-axis) and in vivo Luc expression (RLU, y-axis) for 11 firefly Luc sequence variants containing m¹Ψ, with linear regression line and Pearson correlation.

FIGS. 40A-40D show the inclusion of modified nucleotides in mRNA alters protein expression. FIG. 40A shows the correlation between the GC % of mRNA (x-axis) and eGFP protein production in HeLa cells (y-axis) for unmodified mRNA. FIG. 40B demonstrates the correlation between the GC % of mRNA (x-axis) and hEpo protein production in HeLa cells (y-axis) for unmodified mRNA. FIG. 40C depicts the correlation of secreted hEpo protein production in primary mouse hepatocytes (x-axis) and HeLa cells (y-axis) as measured by ELISA in ng/mL following transfection of cells with 8 sequence variants (described in FIG. 40B above) plus one “codon optimized” variant (E_{CO}) (Welch et al., 2009) containing uridine (left panel), m¹Ψ (middle panel), or mo⁵U (right panel). FIG. 40D shows the correlation of secreted hEpo protein production in primary mouse HeLa cells (right graph) and primary mouse hepatocytes (left graph) to mean serum concentrations (y-axis) of hEpo protein in BALB-c mice following IV injection of LNP-formulated mRNA of 6 sequence variants plus one “codon optimized” variant (E_{CO}) (Welch et al., 2009). Data is shown for mRNA containing m¹Ψ (left panel) and mo⁵U (right panel).

FIGS. 41A-41C show that the inclusion of modified nucleotides in mRNA alters Luc expression. FIG. 41A shows correlations between U % (x-axis, left column), GC % (x-axis, middle column), or codon adaptive index (CAI) (x-axis, right column) vs. Luc expression in HeLa cells (RLU) (y-axis) for 39 Luc sequence variants containing U (top row), m¹Ψ (middle row), and mo⁵U (bottom row), with linear regressions and Pearson correlations. Values are the same as in FIG. 44A. FIG. 41B shows the distribution of expression levels across all variants for each nucleotide as a violin plot with the median (while circle) and inter-quartile range (black lines) of expression values indicated for uridine, m¹Ψ, and mo⁵U. Distribution shown for expression levels in both AML12 cells (top panel) and primary mouse hepatocytes (bottom panel). FIG. 41C shows the correlation of Luc protein production in primary mouse HeLa (right graph) and AML12 (left graph) cells to mean total luminescence of in vivo protein expression (RLU, y-axis) in CD-1 following IV injection of 1.5 mg/kg LNP-formulated mRNA for 10 Luc sequence variants containing m¹Ψ (left panel) or mo⁵U (right panel).

FIG. 42 shows the codon effects of inclusion of modified nucleotides on Luc expression. Grid comparisons of protein expression for 39 Luc sequence variants by global codon usage (rows) for mRNA containing uridine (left grid), m¹Ψ (middle grid), or mo⁵U (right grid) are shown. Each row is ordered by frequency of codons in human genome with the most frequent appearing on the left. Codons for which global usage does not significantly impact protein expression relative to other codons are colored grey. Significant differences by two-way ANOVA comparisons are indicated using lines and the codon with the higher median expression value is colored green. P-values are noted by an increasing number of asterisks for P≤0.05 (*), ≤0.01 (**), ≤0.001 (***), and ≤0.0001 (****).

FIG. 43 shows that mRNA half-life poorly correlates to expression differences. The correlation between the mRNA half-life in AML12 cells (y-axis, taken from the exponential decay lines in C above) and in vivo Luc expression (x-axis, RLU) for 11 variant mRNAs containing m¹Ψ (left) and mo⁵U (right) with linear regression lines and Pearson correlations is shown.

FIGS. 44A-44D demonstrate that the inclusion of modified nucleotides in mRNA alters Luc expression. FIG. 44A, left panel shows the expression in HeLa cells (RLU, y-axis) for 39 firefly Luc sequence variants (L₁ through L₃₉, x-axis) containing uridine (top), m¹Ψ (middle), or mo⁵U (bottom). FIG. 44A, right panel shows the distribution of expression levels across all variants for each nucleotide as a violin plot with the median (while circle) and inter-quartile range (black lines) of expression values indicated for uridine, m¹Ψ, and mo⁵U. FIG. 44B shows a comparison of expression in HeLa cells (RLU) for 39 firefly Luc sequence variants containing m¹Ψ vs. uridine (top), mo⁵U vs. uridine (middle), and m¹Ψ vs. mo⁵U (bottom). Values are the same as in FIG. 44A. FIG. 44C shows the Luc expression in HeLa cells characterized by the codon used for all instances of serine (top), phenylalanine (middle), and threonine (bottom) for 39 Luc sequence variants containing uridine (left), m¹Ψ (middle), or mo⁵U (right). Codons are presented from left to right in order of frequency of occurrence in the human transcriptome. Individual values (dots) with mean and standard errors (black lines). Significant differences by two-way ANOVA comparisons are indicated using lines above each plot, and p-values are noted by an increasing number of asterisks for P≤0.05, ≤0.01, ≤0.001, and ≤0.0001. Values are the same as in FIG. 44A. FIG. 44D shows the total luminescence of in vivo protein expression (RLU, y-axis) in CD-1 mice (five per group) following IV injection of 1.5 mg/kg LNP-formulated mRNA for 10 Luc sequence variants (x-axis) containing m¹Ψ (left) or mo⁵U (right). Individual animals (dots) are shown with the median.

### DETAILED DESCRIPTION

Embodiments of the present disclosure provide synthetic structurally stable RNA (e.g., mRNA). methods of synthesizing the RNA, and methods of delivering the RNA, and its resulting peptide, to a subject.

mRNA-based therapeutics have gained widespread attention as a potential novel clinical platform for treating a wide-array of clinical diseases. Incorporation of modified nucleotides into mRNAs provides a strategy for bypassing components of the innate immune response, but how those modifications impacted the process of protein translation was poorly understood.

The invention relates in some aspects to the mechanism underlying mRNA processing and how those are tied to the structure of mRNA. In order to model how single-atom changes affect bonding between nucleosides and how those impact mRNA expression methods for correlating the structure and function have been developed. An algorithm that predicts, for a given protein, what mRNA sequence would produce the structure that is most appealing to a ribosome and thus most efficiently expressed was developed. In tests of numerous mRNA drug candidates, several structures having a several-fold increase in protein production were observed. New structure design rules were developed for maximizing expression levels.

As shown in the examples, sixty distinct RNAs encoding three unique functional proteins were examined across up to five different chemical modifications in order to develop the first comprehensive picture of how modified nucleotides impact protein translation. This work demonstrates that the chemistry of the nucleotides interacts with the primary sequence of the RNA in order to determine the efficiency of translation. The finding that changing the nucleotide chemistry, but not the primary sequence of the mRNA, changes the process of translation has widespread implications not only for therapeutics based on exogenous RNAs, but also for general principles by which codon changes impact translation.

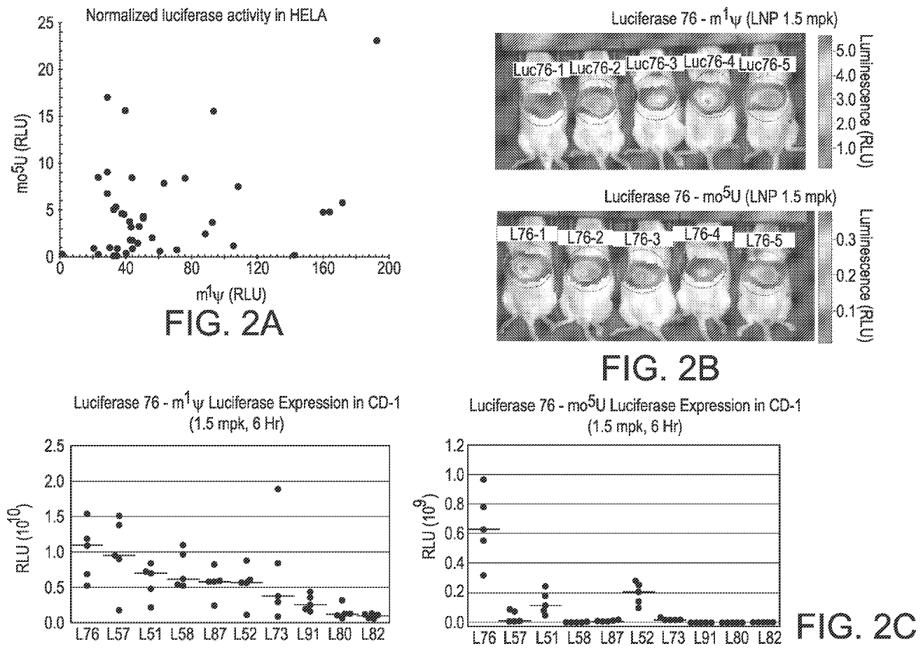

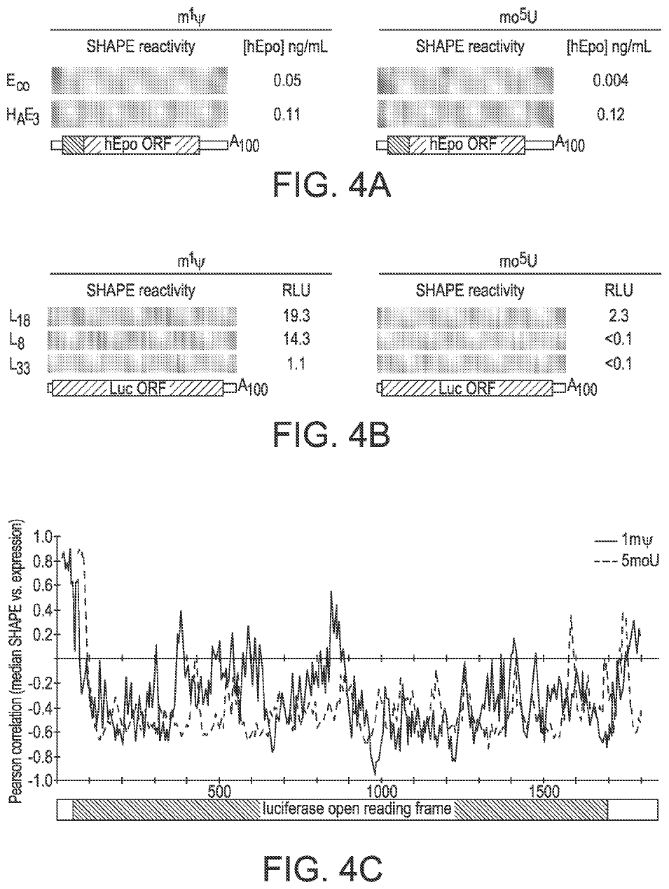

While investigating how the primary sequences of mRNAs translation across multiple nucleotide chemistries, the global structural properties of the mRNA emerged as one of the critical factors influencing translation. Chemical modification had dramatic impact on the thermodynamics of RNA basepairing, often approaching differences of up to 1 kcal/mole for each basepair in the RNA secondary structure (FIG. 2B). These differences combined to give drastic differences in both the thermodynamic stability and the accessible structural conformations of RNAs (FIGS. 2A and 2D). Using single-nucleotide resolution structural probing across a large number of RNAs, a position-dependent, bipartite functional relationship within the mRNA was detected. Highly expressed mRNAs as tested were characterized by a combination of increased flexibility within the 5′ UTR and about the first codons of the open reading frame as well as a general increase in structural stability across the rest of the open reading frame (FIG. 4B). The thermodynamic stability imparted by the modified nucleotides thus synergizes with primary sequence to satisfy these two constraints, with the primary sequence of the mRNA allowing flexibility for stabilizing chemical modifications and imparting stability within the ORF for destabilizing modifications.

The present disclosure demonstrates that the structure of mRNAs directly impacts the process of translation. Chemical modification of the RNA provides a unique opportunity to assay the impact of secondary structure without changing many of the inter-related properties of the mRNA. Surprisingly, the data shown herein demonstrate that secondary structure within the open reading frame enhances protein production by increasing the association of structure mRNAs with polysomes. This directly contradicts current models that suggest secondary structure within the mRNA should decrease protein production by inhibiting of ribosomal processivity. One of the most interesting features of a model where RNA secondary structure is beneficial to translation is the degree of synergy in mRNA regulation.

### Selective 2′-Hydroxyl Acylation Analyzed by Primer Extension (SHAPE)

In some embodiments. RNA structure and flexibility may be analyzed by Selective 2′-Hydroxyl Acylation analyzed by Primer Extension (SHAPE). SHAPE is a technique used to measure flexibility at the single nucleotide level (Smola et al., 2015). Nucleotide sequences are probed with specific SHAPE reagents, which preferentially react with the 2′-hydroxyl groups of conformationally flexible RNA nucleotides, as compared to conformationally constrained RNA nucleotides. SHAPE reagents include, but are not limited to, 1-methyl-7-nitroisatoic anhydride (1M7), 1-methyl-6-nitroisatoic anhydride (1M6), and N-methyl-isatoic anhydride (NMIA). SHAPE reagents also are self-quenching, using a hydrolysis mechanism. The resulting products are analyzed by primer extension using reverse transcription. During this step, polymerase reads through the nucleotides, recording the adduct-induced mutations to be recorded as nucleotide sites non-complementary to the original sequence in the cDNA. The cDNA is then subjected to PCR or second-strand synthesis to construct high-quality libraries for sequencing. The resulting sequencing library then undergoes massively parallel sequencing, and the results are aligned with their respective target sequences. Then, mutation rates can be calculated and SHAPE reactivity profiles may be created. In some embodiments. SHAPE may be used to determine or quantify the flexibility of a given region of a polynucleotide.

In some embodiments, the median SHAPE reactivity of the RNA (e.g., mRNA) is less than 4.0. In some embodiments, the median SHAPE reactivity of the RNA (e.g., mRNA) is within the range of 0.4-0.8, 0.4-1.0, 0.4-1.2, 0.4-1.4, 0.4-1.6, 0.4-1.8, 0.4-2.0, 0.4-2.2, 0.4-2.4, 0.4-2.6, 0.4-2.8, 0.4-3.0, 0.4-0.8, 0.4-1.0, 0.4-1.2, 0.4-1.4, 0.4-1.6, 0.4-1.8, 0.4-2.0, 0.4-2.2, 0.4-2.4, 0.4-2.6, 0.4-2.8, 0.4-3.0, 0.5-0.8, 0.5-1.0, 0.5-1.2, 0.5-1.4, 0.5-1.6, 0.5-1.8, 0.5-2.0, 0.5-2.2, 0.5-2.4, 0.5-2.6, 0.5-2.8, 0.5-3.0, 0.6-0.8, 0.6-1.0, 0.6-1.2, 0.6-1.4, 0.6-1.6, 0.6-1.8, 0.6-2.0, 0.6-2.2, 0.6-2.4, 0.6-2.6, 0.6-2.8, 0.6-3.0, 0.7-0.8, 0.7-1.0, 0.7-1.2, 0.7-1.4, 0.7-1.6, 0.7-1.8, 0.7-2.0, 0.7-2.2, 0.7-2.4, 0.7-2.6, 0.7-2.8, 0.7-3.0, 0.8-1.0, 0.8-1.2, 0.8-1.4, 0.8-1.6, 0.8-1.8, 0.8-2.0, 0.8-2.2, 0.8-2.4, 0.8-2.6, 0.8-2.8, 0.8-3.0, 0.9-1.0, 0.9-1.2, 0.9-1.4.0.9-1.6, 0.9-1.8, 0.9-2.0, 0.9-2.2, 0.9-2.4, 0.9-2.6, 0.9-2.8, 0.9-3.0, 1.0-1.5, 1.0-2.0, 1.5-2.5, 1.5-3.0, 1.5-3.5, 1.5-4.0, 2.0-2.5, 2.5-3.0, 2.5-3.5, 2.5-4.0, 3.0-3.5, 3.5-4.0. In some embodiments, the median SHAPE reactivity of the RNA (e.g., mRNA) is less than 3.8, less than 3.6, less than 3.4, less than 3.2, less than 3.0, less than 2.8, less than 2.6, less than 2.4, less than 2.2, less than 2.0, less than 1.8, less than 1.6, less than 1.4, less than 1.2, less than 1.0, less than 0.8, less than 0.6, or less than 0.4, for example. In some embodiments, the RNA (e.g., mRNA) has a first flexible region with a relatively higher SHAPE reactivity score and a second, more constrained region, as evidenced by a lower SHAPE reactivity score. In some embodiments, the flexible first region of the RNA may include the 5′ UTR as well as the first 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides of the open reading frame (ORF). In further embodiments, the structured second region of the RNA may include the entire ORF, or less than the entire ORF, as well as the 3′ UTR.

### Thermodynamics and UV-Melting Analysis

In some embodiments, the RNA of the present disclosure may be analyzed according to thermodynamic properties. In some embodiments, the primary sequence is thermodynamically unstable. In other embodiments, the primary sequence is thermodynamically stable. Polynucleotides have innate thermodynamic stability or instability, owing to their specific nucleotide chemistry. In some embodiments, the incorporation of modified nucleotides may alter the innate thermodynamic stability. In some embodiments, global thermostability is measured using UV-melting analysis. The RNA is heated, and the normalized first derivative of the UV-absorbance quantifies the amount of RNA structure that melts at a given temperature.

In some embodiments, greater than 50% of the thermostable mRNA forms secondary structure at 37° C. In other embodiments, the percentage of the thermostable mRNA forming secondary structure at 37° C. is 55%, 60%, 65%, 70%, 72%, 74%, 75%, 76%, 78%, 80%, 82%, 84%, 85%, 86%, 88%, 90%, 92%, 94%, 95%, 96%, 98%, 99%, or 100%. In still other embodiments, the polynucleotide may contain any percentage of thermostable mRNA (e.g., from 50% to 60%, from 50% to 70%, from 50% to 80%, from 50% to 90%, from 50% to 95%, from 505 to 100%, from 60% to 70%, from 60% to 80%, from 60% to 90%, from 60% to 95%, from 60% to 100%, from 70% to 80%, from 70% to 90%, from 70% to 95%, from 70% to 100%, from 80% to 90%, from 80% to 95%, from 80% to 100%, from 85% to 90%, from 85% to 95%, from 85% to 100%, from 90% to 95%, and from 95% to 100%).

In other embodiments, the 5′ region of the mRNA (the flexible region) is more flexible than the subsequent open reading frame (ORF) and 3′ UTR (the structurally stable region). The 5′ region may include the first 5, 6, 7, 8, 9, 10, 12, 14, 16, 18, 20, 22, 24, 26, 28, 30, 32, 34, 36, 38, 40, 45, 50, 55, 60, 65, or 70 nucleotides of the 5′ end of the ORF and the 5′ UTR. It is understood that the remaining ORF nucleotides together with the 3′ UTR form the structurally stable region.

In some embodiments, less than 30% of the flexible 5′ region may form secondary structure at 37° C., as defined by UV-melting analysis. In other embodiments, the percentage of thermostable mRNA forming secondary structure at 37° C. in the flexible 5′ region is 1%, 2%, 3%, 4%, 5%, 6%, 7%, 8%, 9%, 10%, 12%, 14%, 16%, 18%, 20%, 22%, 24%, 26%, 28%, 30%, 32%, 34%, 36%, 38%, 40%, or 45%. In still other embodiments, the flexible 5′ region may contain any percentage of thermostable mRNA (e.g., from 1% to 20%, from 1% to 25%, from 1% to 50%, from 5% to 20%, from 5% to 25%, from 5% to 50%, from 10% to 20%, from 10% to 25%, from 10% to 50%, from 20% to 25%, from 20% to 50%, from 30% to 40%, from 30% to 50%, and from 40% to 45%).

In some embodiments, greater than 50% of the structurally stable mRNA region forms secondary structure at 37° C. In other embodiments, the percentage of the thermostable mRNA of the structurally stable region forming secondary structure at 37° C. is 55%, 60%, 65%, 70%, 72%, 74%, 75%, 76%, 78%, 80%, 82%, 84%, 85%, 86%, 88%, 90%, 92%, 94%, 95%, 96%, 98%, 99%, or 100%. In still other embodiments, the structurally stable region may contain any percentage of thermostable mRNA (e.g., from 50% to 60%, from 50% to 70%, from 50% to 80%, from 50% to 90%, from 50% to 95%, from 505 to 100%, from 60% to 70%, from 60% to 80%, from 60% to 90%, from 60% to 95%, from 60% to 100%, from 70% to 80%, from 70% to 90%, from 70% to 95%, from 70% to 100%, from 80% to 90%, from 80% to 95%, from 80% to 100%, from 85% to 90%, from 85% to 95%, from 85% to 100%, from 90% to 95%, and from 95% to 100%).

### Minimum Free Energy and Synonymous Variants

In some embodiments, the RNA of the present disclosure has a minimum free energy (MFE) value less than that of a median distribution MFE value of synonymous variants. The MFE indicates the lowest free energy value secondary structure of a given sequence. Generally, lower MFE values represent more thermodynamically stable structures, as stabilizing structures, such as Watson-Crick base pairs, yield negative free energy, while destabilizing structures, such as unpaired bases and destabilizing loops have positive free energy. Synonymous variants are nucleotide sequences containing one or more nucleotide substitutions that do not change the amino acid sequence of the resulting protein.

In some embodiments, the RNA of the present disclosure has a MFE value within the top 0.1% of low MFE, as defined computationally of synonymous variants. In other embodiments, the RNA of the present disclosure has a MFE value within the top 1%, 0.9%, 0.8%, 0.7%, 0.6%, 0.5%, 0.4%, 0.3%, 0.2%, 0.05%, or 0.01% of low MFE, as defined computationally of synonymous variants.

### Nucleic Acids/Polynucleotides

Nucleic acids (also referred to as polynucleotides) may be or may include, for example, RNAs, deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) or chimeras or combinations thereof.

In some embodiments, polynucleotides of the present disclosure function as messenger RNA (mRNA). “Messenger RNA” (mRNA) refers to any polynucleotide that encodes a (at least one) polypeptide (a naturally-occurring, non-naturally-occurring, or modified polymer of amino acids) and can be translated to produce the encoded polypeptide in vitro, in vivo, in situ or ex vivo.

The basic components of an mRNA molecule typically include at least one coding region, a 5′ untranslated region (UTR), a 3′ UTR, a 5′ cap and a poly-A tail. Polynucleotides of the present disclosure may function as mRNA but can be distinguished from wild-type mRNA in their functional and/or structural design features which serve to overcome existing problems of effective polypeptide expression using nucleic-acid based therapeutics.

Polynucleotides of the present disclosure, in some embodiments, are codon optimized. Codon optimization methods are known in the art and may be used as provided herein. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g. glycosylation sites); add, remove or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. Codon optimization tools, algorithms and services are known in the art—non-limiting examples include services from GeneArt (Life Technologies), DNA2.0 (Menlo Park Calif.) and/or proprietary methods. In some embodiments, the open reading frame (ORF) sequence is optimized using optimization algorithms.

In some embodiments, a codon optimized sequence shares less than 95% sequence identity to a naturally-occurring or wild-type sequence (e.g., a naturally-occurring or wild-type mRNA sequence encoding a polypeptide or protein of interest (e.g., an antigenic protein or polypeptide. In some embodiments, a codon optimized sequence shares less than 90% sequence identity to a naturally-occurring or wild-type sequence (e.g., a naturally-occurring or wild-type mRNA sequence encoding a polypeptide or protein of interest (e.g., an antigenic protein or polypeptide. In some embodiments, a codon optimized sequence shares less than 85% sequence identity to a naturally-occurring or wild-type sequence (e.g., a naturally-occurring or wild-type mRNA sequence encoding a polypeptide or protein of interest (e.g., an antigenic protein or polypeptide. In some embodiments, a codon optimized sequence shares less than 80% sequence identity to a naturally-occurring or wild-type sequence (e.g., a naturally-occurring or wild-type mRNA sequence encoding a polypeptide or protein of interest (e.g., an antigenic protein or polypeptide. In some embodiments, a codon optimized sequence shares less than 75% sequence identity to a naturally-occurring or wild-type sequence (e.g., a naturally-occurring or wild-type mRNA sequence encoding a polypeptide or protein of interest (e.g., an antigenic protein or polypeptide).

In some embodiments, a codon optimized sequence shares between 65% and 85% (e.g., between about 67% and about 85% or between about 67% and about 80%) sequence identity to a naturally-occurring or wild-type sequence (e.g., a naturally-occurring or wild-type mRNA sequence encoding a polypeptide or protein of interest (e.g., an antigenic protein or polypeptide. In some embodiments, a codon optimized sequence shares between 65% and 75 or about 80% sequence identity to a naturally-occurring or wild-type sequence (e.g., a naturally-occurring or wild-type mRNA sequence encoding a polypeptide or protein of interest (e.g., an antigenic protein or polypeptide).

In some embodiments a codon optimized RNA may, for instance, be one in which the levels of G/C are enhanced. The G/C-content of nucleic acid molecules may influence the stability of the RNA. RNA having an increased amount of guanine (G) and/or cytosine (C) residues may be functionally more stable than nucleic acids containing a large amount of adenine (A) and thymine (T) or uracil (U) nucleotides. WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

### Chemical Modifications

Structurally stable RNA (e.g., mRNA) of the present disclosure may comprise at least one ribonucleic acid (RNA) polynucleotide having an open reading frame that comprises at least one chemical modification.

In some embodiments, nucleotides and nucleosides of the present disclosure comprise modified nucleotides or nucleosides. Such modified nucleotides and nucleosides can be naturally-occurring modified nucleotides and nucleosides or non-naturally occurring modified nucleotides and nucleosides. Such modifications can include those at the sugar, backbone, or nucleobase portion of the nucleotide and/or nucleoside as are recognized in the art.

In some embodiments, a naturally-occurring modified nucleotide or nucleotide of the disclosure is one as is generally known or recognized in the art. Non-limiting examples of such naturally occurring modified nucleotides and nucleotides can be found, inter alia, in the widely recognized MODOMICS database.

In some embodiments, a non-naturally occurring modified nucleotide or nucleoside of the disclosure is one as is generally known or recognized in the art. Non-limiting examples of such non-naturally occurring modified nucleotides and nucleosides can be found, inter alia, in published US application Nos. PCT/US2012/058519; PCT/US2013/075177; PCT/US2014/058897; PCT/US2014/058891; PCT/US2014/070413; PCT/US2015/36773; PCT/US2015/36759; PCT/US2015/36771; or PCT/IB2017/051367 all of which are incorporated by reference herein.

Hence, nucleic acids of the disclosure (e.g., DNA nucleic acids and RNA nucleic acids, such as mRNA nucleic acids) can comprise standard nucleotides and nucleosides, naturally-occurring nucleotides and nucleosides, non-naturally-occurring nucleotides and nucleosides, or any combination thereof.

Nucleic acids of the disclosure (e.g., DNA nucleic acids and RNA nucleic acids, such as mRNA nucleic acids), in some embodiments, comprise various (more than one) different types of standard and/or modified nucleotides and nucleosides. In some embodiments, a particular region of a nucleic acid contains one, two or more (optionally different) types of standard and/or modified nucleotides and nucleosides.

In some embodiments, a modified RNA nucleic acid (e.g., a modified mRNA nucleic acid), introduced to a cell or organism, exhibits reduced degradation in the cell or organism, respectively, relative to an unmodified nucleic acid comprising standard nucleotides and nucleosides.

In some embodiments, a modified RNA nucleic acid (e.g., a modified mRNA nucleic acid), introduced into a cell or organism, may exhibit reduced immunogenicity in the cell or organism, respectively (e.g., a reduced innate response) relative to an unmodified nucleic acid comprising standard nucleotides and nucleosides.

Nucleic acids (e.g., RNA nucleic acids, such as mRNA nucleic acids), in some embodiments, comprise non-natural modified nucleotides that are introduced during synthesis or post-synthesis of the nucleic acids to achieve desired functions or properties. The modifications may be present on internucleotide linkages, purine or pyrimidine bases, or sugars. The modification may be introduced with chemical synthesis or with a polymerase enzyme at the terminal of a chain or anywhere else in the chain. Any of the regions of a nucleic acid may be chemically modified.

The present disclosure provides for modified nucleosides and nucleotides of a nucleic acid (e.g., RNA nucleic acids, such as mRNA nucleic acids). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Nucleic acids can comprise a region or regions of linked nucleosides. Such regions may have variable backbone linkages. The linkages can be standard phosphodiester linkages, in which case the nucleic acids would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures, such as, for example, in those nucleic acids having at least one chemical modification. One example of such non-standard base pairing is the base pairing between the modified nucleotide inosine and adenine, cytosine or uracil. Any combination of base/sugar or linker may be incorporated into nucleic acids of the present disclosure.

In some embodiments, modified nucleobases in nucleic acids (e.g., RNA nucleic acids, such as mRNA nucleic acids) comprise 1-methyl-pseudouridine (m1ψ), 1-ethyl-pseudouridine (e1ψ), 5-methoxy-uridine (mo5U), 5-methyl-cytidine (m5C), and/or pseudouridine (ψ). In some embodiments, modified nucleobases in nucleic acids (e.g., RNA nucleic acids, such as mRNA nucleic acids) comprise 5-methoxymethyl uridine, 5-methylthio uridine, 1-methoxymethyl pseudouridine, 5-methyl cytidine, and/or 5-methoxy cytidine. In some embodiments, the polyribonucleotide includes a combination of at least two (e.g., 2, 3, 4 or more) of any of the aforementioned modified nucleobases, including but not limited to chemical modifications.

In some embodiments, a RNA nucleic acid of the disclosure comprises 1-methyl-pseudouridine (m1ψ) substitutions at one or more or all uridine positions of the nucleic acid.

In some embodiments, a RNA nucleic acid of the disclosure comprises 1-methyl-pseudouridine (m1ψ) substitutions at one or more or all uridine positions of the nucleic acid and 5-methyl cytidine substitutions at one or more or all cytidine positions of the nucleic acid.

In some embodiments, a RNA nucleic acid of the disclosure comprises pseudouridine (ψ) substitutions at one or more or all uridine positions of the nucleic acid.

In some embodiments, a RNA nucleic acid of the disclosure comprises pseudouridine (ψ) substitutions at one or more or all uridine positions of the nucleic acid and 5-methyl cytidine substitutions at one or more or all cytidine positions of the nucleic acid.

In some embodiments, a RNA nucleic acid of the disclosure comprises uridine at one or more or all uridine positions of the nucleic acid.

In some embodiments, nucleic acids (e.g., RNA nucleic acids, such as mRNA nucleic acids) are uniformly modified (e.g., fully modified, modified throughout the entire sequence) for a particular modification. For example, a nucleic acid can be uniformly modified with 1-methyl-pseudouridine, meaning that all uridine residues in the mRNA sequence are replaced with 1-methyl-pseudouridine. Similarly, a nucleic acid can be uniformly modified for any type of nucleoside residue present in the sequence by replacement with a modified residue such as those set forth above.

The nucleic acids of the present disclosure may be partially or fully modified along the entire length of the molecule. For example, one or more or all or a given type of nucleotide (e.g., purine or pyrimidine, or any one or more or all of A, G, U, C) may be uniformly modified in a nucleic acid of the disclosure, or in a predetermined sequence region thereof (e.g., in the mRNA including or excluding the polyA tail). In some embodiments, all nucleotides X in a nucleic acid of the present disclosure (or in a sequence region thereof) are modified nucleotides, wherein X may be any one of nucleotides A, G, U, C, or any one of the combinations A+G, A+U. A+C, G+U, G+C, U+C, A+G+U, A+G+C, G+U+C or A+G+C.

The nucleic acid may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The nucleic acids may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as a modified uracil or cytosine. In some embodiments, at least 5%, at least 10%, at least 25%, at least 50%, at least 80%, at least 90% or 100% of the uracil in the nucleic acid is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the nucleic acid is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

Thus, in some embodiments, the RNA (e.g., mRNA) comprises a 5′UTR element, an optionally codon optimized open reading frame, and a 3′UTR element, a poly(A) sequence and/or a polyadenylation signal wherein the RNA is not chemically modified.

In some embodiments, the mRNA of the present disclosure is highly expressing. Highly expressing mRNA means that the mRNA expresses more protein relative to a corresponding wild-type chemically unmodified RNA. In some embodiments, the highly expressing mRNA produces at least 10% more protein than the wild-type RNA. In other embodiments, the highly expressing mRNA produces at least 5%, at least 15%, at least 20%, at least 25%, at least 30%, at least 35%, at least 40%, at least 45%, at least 50%, at least 60%, at least 70%, at least 80%, at least 90%, at least 100% or at least 110% more protein than wild-type RNA.

In Vitro Transcription of RNA (e.g., mRNA)

Structurally stable polynucleotides of the present disclosure comprise at least one RNA polynucleotide, such as an mRNA (e.g., modified mRNA). mRNA, for example, is transcribed in vitro from template DNA, referred to as an “in vitro transcription template.” In some embodiments, an in vitro transcription template encodes a 5′ untranslated (UTR) region, contains an open reading frame, and encodes a 3′ UTR and a polyA tail. The particular nucleic acid sequence composition and length of an in vitro transcription template will depend on the mRNA encoded by the template.

In some embodiments, a polynucleotide includes 200 to 3.000 nucleotides. For example, a polynucleotide may include 200 to 500, 200 to 1000, 200 to 1500, 200 to 3000, 500 to 1000, 500 to 1500, 500 to 2000, 500 to 3000, 1000 to 1500, 1000 to 2000, 1000 to 3000, 1500 to 3000, or 2000 to 3000 nucleotides).

In other aspects, the invention relates to a method for preparing an RNA composition by IVT methods. In vitro transcription (IVT) methods permit template-directed synthesis of RNA molecules of almost any sequence. The size of the RNA molecules that can be synthesized using IVT methods range from short oligonucleotides to long nucleic acid polymers of several thousand bases. IVT methods permit synthesis of large quantities of RNA transcript (e.g., from microgram to milligram quantities) (Beckert et al., Synthesis of RNA by in vitro transcription, Methods Mol Biol. 703:29-41(2011); Rio et al. RNA: A Laboratory Manual. Cold Spring Harbor: Cold Spring Harbor Laboratory Press. 2011, 205-220.: Cooper. Geoffery M. The Cell: A Molecular Approach. 4th ed. Washington D.C.: ASM Press. 2007, 262-299). Generally, IVT utilizes a DNA template featuring a promoter sequence upstream of a sequence of interest. The promoter sequence is most commonly of bacteriophage origin (ex, the T7, T3 or SP6 promoter sequence) but many other promotor sequences can be tolerated including those designed de novo. Transcription of the DNA template is typically best achieved by using the RNA polymerase corresponding to the specific bacteriophage promoter sequence. Exemplary RNA polymerases include, but are not limited to T7 RNA polymerase, T3 RNA polymerase, or SP6 RNA polymerase, among others. IVT is generally initiated at a dsDNA but can proceed on a single strand.

It will be appreciated that immunomodulatory therapeutic compositions of the present disclosure, e.g., mRNAs encoding the activating oncogene mutation peptide, may be made using any appropriate synthesis method. For example, in some embodiments, immunomodulatory therapeutic compositions of the present disclosure are made using IVT from a single bottom strand DNA as a template and complementary oligonucleotide that serves as promotor. The single bottom strand DNA may act as a DNA template for in vitro transcription of RNA, and may be obtained from, for example, a plasmid, a PCR product, or chemical synthesis. In some embodiments, the single bottom strand DNA is linearized from a circular template. The single bottom strand DNA template generally includes a promoter sequence, e.g., a bacteriophage promoter sequence, to facilitate IVT. Methods of making RNA using a single bottom strand DNA and a top strand promoter complementary oligonucleotide are known in the art. An exemplary method includes, but is not limited to, annealing the DNA bottom strand template with the top strand promoter complementary oligonucleotide (e.g., T7 promoter complementary oligonucleotide. T3 promoter complementary oligonucleotide, or SP6 promoter complementary oligonucleotide), followed by IVT using an RNA polymerase corresponding to the promoter sequence, e.g., aT7 RNA polymerase, a T3 RNA polymerase, or an SP6 RNA polymerase.

IVT methods can also be performed using a double-stranded DNA template. For example, in some embodiments, the double-stranded DNA template is made by extending a complementary oligonucleotide to generate a complementary DNA strand using strand extension techniques available in the art. In some embodiments, a single bottom strand DNA template containing a promoter sequence and sequence encoding one or more epitopes of interest is annealed to a top strand promoter complementary oligonucleotide and subjected to a PCR-like process to extend the top strand to generate a double-stranded DNA template. Alternatively or additionally, a top strand DNA containing a sequence complementary to the bottom strand promoter sequence and complementary to the sequence encoding one or more epitopes of interest is annealed to a bottom strand promoter oligonucleotide and subjected to a PCR-like process to extend the bottom strand to generate a double-stranded DNA template. In some embodiments, the number of PCR-like cycles ranges from 1 to 20 cycles. e.g., 3 to 10 cycles. In some embodiments, a double-stranded DNA template is synthesized wholly or in part by chemical synthesis methods. The double-stranded DNA template can be subjected to in vitro transcription as described herein.

In another aspect, immunomodulatory therapeutic compositions of the present disclosure, e.g., mRNAs encoding the activating oncogene mutation peptide, may be made using two DNA strands that are complementary across an overlapping portion of their sequence, leaving single-stranded overhangs (i.e., sticky ends) when the complementary portions are annealed. These single-stranded overhangs can be made double-stranded by extending using the other strand as a template, thereby generating double-stranded DNA. In some cases, this primer extension method can permit larger ORFs to be incorporated into the template DNA sequence, e.g., as compared to sizes incorporated into the template DNA sequences obtained by top strand DNA synthesis methods. In the primer extension method, a portion of the 3′-end of a first strand (in the 5″-3′ direction) is complementary to a portion the 3′-end of a second strand (in the 3′-5′ direction). In some such embodiments, the single first strand DNA may include a sequence of a promoter (e.g., T7, T3, or SP6), optionally a 5′-UTR, and some or all of an ORF (e.g., a portion of the 5′-end of the ORF). In some embodiments, the single second strand DNA may include complementary sequences for some or all of an ORF (e.g., a portion complementary to the 3′-end of the ORF), and optionally a 3′-UTR, a stop sequence, and/or a poly(A) tail. Methods of making RNA using two synthetic DNA strands may include annealing the two strands with overlapping complementary portions, followed by primer extension using one or more PCR-like cycles to extend the strands to generate a double-stranded DNA template. In some embodiments, the number of PCR-like cycles ranges from 1 to 20 cycles. e.g., 3 to 10 cycles. Such double-stranded DNA can be subjected to in vitro transcription as described herein.

In another aspect, RNA compositions of the present disclosure, e.g., chemically-modified mRNAs, may be made using synthetic double-stranded linear DNA molecules, such as gBlocks® (Integrated DNA Technologies, Coralville, Iowa), as the double-stranded DNA template. An advantage to such synthetic double-stranded linear DNA molecules is that they provide a longer template from which to generate mRNAs. For example, gBlocks® can range in size from 45-1000 (e.g., 125-750 nucleotides). In some embodiments, a synthetic double-stranded linear DNA template includes a full length 5′-UTR, a full length 3′-UTR, or both. A full length 5′-UTR may be up to 100 nucleotides in length, e.g., about 40-60 nucleotides. A full length 3′-UTR may be up to 300 nucleotides in length, e.g., about 100-150 nucleotides.

To facilitate generation of longer constructs, two or more double-stranded linear DNA molecules and/or gene fragments that are designed with overlapping sequences on the 3′ strands may be assembled together using methods known in art. For example, the Gibson Assembly™ Method (Synthetic Genomics, Inc., La Jolla, Calif.) may be performed with the use of a mesophilic exonuclease that cleaves bases from the 5′-end of the double-stranded DNA fragments, followed by annealing of the newly formed complementary single-stranded 3′-ends, polymerase-dependent extension to fill in any single-stranded gaps, and finally, covalent joining of the DNA segments by a DNA ligase.

In another aspect, immunomodulatory therapeutic compositions of the present disclosure, e.g., mRNAs encoding the activating oncogene mutation peptide, may be made using chemical synthesis of the RNA. Methods, for instance, involve annealing a first polynucleotide comprising an open reading frame encoding the polypeptide and a second polynucleotide comprising a 5′-UTR to a complementary polynucleotide conjugated to a solid support. The 3′-terminus of the second polynucleotide is then ligated to the 5′-terminus of the first polynucleotide under suitable conditions. Suitable conditions include the use of a DNA Ligase. The ligation reaction produces a first ligation product. The 5′ terminus of a third polynucleotide comprising a 3′-UTR is then ligated to the 3′-terminus of the first ligation product under suitable conditions. Suitable conditions for the second ligation reaction include an RNA Ligase. A second ligation product is produced in the second ligation reaction. The second ligation product is released from the solid support to produce an mRNA encoding a polypeptide of interest. In some embodiments the mRNA is between 30 and 1000 nucleotides.

An mRNA encoding a polypeptide of interest may also be prepared by binding a first polynucleotide comprising an open reading frame encoding the polypeptide to a second polynucleotide comprising 3′-UTR to a complementary polynucleotide conjugated to a solid support. The 5′-terminus of the second polynucleotide is ligated to the 3′-terminus of the first polynucleotide under suitable conditions. The suitable conditions include a DNA Ligase. The method produces a first ligation product. A third polynucleotide comprising a 5′-UTR is ligated to the first ligation product under suitable conditions to produce a second ligation product. The suitable conditions include an RNA Ligase, such as T4 RNA. The second ligation product is released from the solid support to produce an mRNA encoding a polypeptide of interest.

In some embodiments the first polynucleotide features a 5′-triphosphate and a 3′-OH. In other embodiments the second polynucleotide comprises a 3′-OH. In yet other embodiments, the third polynucleotide comprises a 5′-triphosphate and a 3′-OH. The second polynucleotide may also include a 5′-cap structure. The method may also involve the further step of ligating a fourth polynucleotide comprising a poly-A region at the 3′-terminus of the third polynucleotide. The fourth polynucleotide may comprise a 5′-triphosphate.

The method may or may not comprise reverse phase purification. The method may also include a washing step wherein the solid support is washed to remove unreacted polynucleotides. The solid support may be, for instance, a capture resin. In some embodiments the method involves dT purification.

In accordance with the present disclosure, template DNA encoding the compositions of the present disclosure includes an open reading frame (ORF) encoding one or more target peptides. In some embodiments, the template DNA includes an ORF of up to 1000 nucleotides, e.g., about 10-350, 30-300 nucleotides or about 50-250 nucleotides. In some embodiments, the template DNA includes an ORF of about 150 nucleotides. In some embodiments, the template DNA includes an ORF of about 200 nucleotides.

In some embodiments. IVT transcripts are purified from the components of the IVT reaction mixture after the reaction takes place. For example, the crude IVT mix may be treated with RNase-free DNase to digest the original template. The mRNA can be purified using methods known in the art, including but not limited to, precipitation using an organic solvent or column based purification method. Commercial kits are available to purify RNA, e.g., MEGACLEAR™ Kit (Ambion, Austin, Tex.). The mRNA can be quantified using methods known in the art, including but not limited to, commercially available instruments. e.g., NanoDrop. Purified mRNA can be analyzed, for example, by agarose gel electrophoresis to confirm the RNA is the proper size and/or to confirm that no degradation of the RNA has occurred.

### Untranslated Regions (UTRs)

A “5′ untranslated region” (UTR) refers to a region of an mRNA that is directly upstream (i.e., 5′) from the start codon (i.e., the first codon of an mRNA transcript translated by a ribosome) that does not encode a polypeptide.

A “3′ untranslated region” (UTR) refers to a region of an mRNA that is directly downstream (i.e., 3′) from the stop codon (i.e., the codon of an mRNA transcript that signals a termination of translation) that does not encode a polypeptide.

An “open reading frame” is a continuous stretch of DNA beginning with a start codon (e.g., methionine (ATG)), and ending with a stop codon (e.g., TAA, TAG or TGA) and encodes a polypeptide.

A “polyA tail” is a region of mRNA that is downstream, e.g., directly downstream (i.e., 3′), from the 3′ UTR that contains multiple, consecutive adenosine monophosphates. A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, export of the mRNA from the nucleus and translation.

In some embodiments, a polynucleotide includes 200 to 3,000 nucleotides. For example, a polynucleotide may include 200 to 500, 200 to 1000, 200 to 1500, 200 to 3000, 500 to 1000, 500 to 1500, 500 to 2000, 500 to 3000, 1000 to 1500, 1000 to 2000, 1000 to 3000, 1500 to 3000, or 2000 to 3000 nucleotides).

### Stabilizing Elements

Naturally-occurring eukaryotic mRNA molecules have been found to contain stabilizing elements, including, but not limited to untranslated regions (UTR) at their 5′-end (5′UTR) and/or at their 3′-end (3′UTR), in addition to other structural features, such as a 5′-cap structure or a 3′-poly(A) tail. Both the 5′UTR and the 3′UTR are typically transcribed from the genomic DNA and are elements of the premature mRNA. Characteristic structural features of mature mRNA, such as the 5′-cap and the 3′-poly(A) tail are usually added to the transcribed (premature) mRNA during mRNA processing. The 3′-poly(A) tail is typically a stretch of adenine nucleotides added to the 3′-end of the transcribed mRNA. It can comprise up to about 400 adenine nucleotides. In some embodiments the length of the 3′-poly(A) tail may be an essential element with respect to the stability of the individual mRNA.

In some embodiments the RNA may include one or more stabilizing elements. Stabilizing elements may include for instance a histone stem-loop. A stem-loop binding protein (SLBP), a 32 kDa protein has been identified. It is associated with the histone stem-loop at the 3′-end of the histone messages in both the nucleus and the cytoplasm. Its expression level is regulated by the cell cycle; it is peaks during the S-phase, when histone mRNA levels are also elevated. The protein has been shown to be essential for efficient 3′-end processing of histone pre-mRNA by the U7 snRNP. SLBP continues to be associated with the stem-loop after processing, and then stimulates the translation of mature histone mRNAs into histone proteins in the cytoplasm. The RNA binding domain of SLBP is conserved through metazoa and protozoa; its binding to the histone stem-loop depends on the structure of the loop. The minimum binding site includes at least three nucleotides 5′ and two nucleotides 3′ relative to the stem-loop.

In some embodiments, the RNA include a coding region, at least one histone stem-loop, and optionally, a poly(A) sequence or polyadenylation signal. The poly(A) sequence or polyadenylation signal generally should enhance the expression level of the encoded protein. The encoded protein, in some embodiments, is not a histone protein, a reporter protein (e.g. Luciferase. GFP, EGFP, β-Galactosidase, EGFP), or a marker or selection protein (e.g. alpha-Globin, Galactokinase and Xanthine:guanine phosphoribosyl transferase (GPT)).

In some embodiments, the combination of a poly(A) sequence or polyadenylation signal and at least one histone stem-loop, even though both represent alternative mechanisms in nature, acts synergistically to increase the protein expression beyond the level observed with either of the individual elements. It has been found that the synergistic effect of the combination of poly(A) and at least one histone stem-loop does not depend on the order of the elements or the length of the poly(A) sequence.

In some embodiments, the RNA does not comprise a histone downstream element (HDE). “Histone downstream element” (HDE) includes a purine-rich polynucleotide stretch of approximately 15 to 20 nucleotides 3′ of naturally occurring stem-loops, representing the binding site for the U7 snRNA, which is involved in processing of histone pre-mRNA into mature histone mRNA.

In some embodiments, the RNA of the present disclosure may or may not contain an enhancer and/or promoter sequence, which may be modified or unmodified or which may be activated or inactivated. In some embodiments, the histone stem-loop is generally derived from histone genes, and includes an intramolecular base pairing of two neighbored partially or entirely reverse complementary sequences separated by a spacer, consisting of a short sequence, which forms the loop of the structure. The unpaired loop region is typically unable to base pair with either of the stem loop elements. It occurs more often in RNA, as is a key component of many RNA secondary structures, but may be present in single-stranded DNA as well. Stability of the stem-loop structure generally depends on the length, number of mismatches or bulges, and base composition of the paired region. In some embodiments, wobble base pairing (non-Watson-Crick base pairing) may result. In some embodiments, the at least one histone stem-loop sequence comprises a length of 15 to 45 nucleotides.

In other embodiments the RNA may have one or more AU-rich sequences removed. These sequences, sometimes referred to as AURES are destabilizing sequences found in the 3′UTR. The AURES may be removed from the RNA. Alternatively the AURES may remain in the RNA.

### Lipid Nanoparticles (LNPs)

In some embodiments, RNA (e.g., mRNA) of the disclosure are formulated in a lipid nanoparticle (LNP). Lipid nanoparticles typically comprise ionizable cationic lipid, non-cationic lipid, sterol and PEG lipid components along with the nucleic acid cargo of interest. The lipid nanoparticles of the disclosure can be generated using components, compositions, and methods as are generally known in the art, see for example PCT/US2016/052352; PCT/US2016/068300; PCT/US2017/037551; PCT/US2015/027400; PCT/US2016/047406; PCT/US2016000129; PCT/US2016/014280: PCT/US2016/014280; PCT/US2017/038426; PCT/US2014/027077; PCT/US2014/055394; PCT/US2016/52117; PCT/US2012/069610; PCT/US2017/027492; PCT/US2016/059575 and PCT/US2016/069491 all of which are incorporated by reference herein in their entirety.

RNA of the present disclosure may be formulated in lipid nanoparticle. In some embodiments, the lipid nanoparticle comprises at least one ionizable cationic lipid, at least one non-cationic lipid, at least one sterol, and/or at least one polyethylene glycol (PEG)-modified lipid.

In some embodiments, the lipid nanoparticle comprises a molar ratio of 20-60% ionizable cationic lipid. For example, the lipid nanoparticle may comprise a molar ratio of 20-50%, 20-40%, 20-30%, 30-60%, 30-50%, 30-40%, 40-60%, 40-50%, or 50-60% ionizable cationic lipid. In some embodiments, the lipid nanoparticle comprises a molar ratio of 20%, 30%, 40%, 50, or 60% ionizable cationic lipid.

In some embodiments, the lipid nanoparticle comprises a molar ratio of 5-25% non-cationic lipid. For example, the lipid nanoparticle may comprise a molar ratio of 5-20%, 5-15%, 5-10%, 10-25%, 10-20%, 10-25%, 15-25%, 15-20%, or 20-25% non-cationic lipid. In some embodiments, the lipid nanoparticle comprises a molar ratio of 5%, 10%, 15%, 20%, or 25% non-cationic lipid.

In some embodiments, the lipid nanoparticle comprises a molar ratio of 25-55% sterol. For example, the lipid nanoparticle may comprise a molar ratio of 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% sterol. In some embodiments, the lipid nanoparticle comprises a molar ratio of 25%, 30%, 35%, 40%, 45%, 50%, or 55% sterol.

In some embodiments, the lipid nanoparticle comprises a molar ratio of 0.5-15% PEG-modified lipid. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10-15%. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG-modified lipid.

In some embodiments, the lipid nanoparticle comprises a molar ratio of 20-60% ionizable cationic lipid, 5-25% non-cationic lipid, 25-55% sterol, and 0.5-15% PEG-modified lipid.

In some embodiments, an ionizable cationic lipid of the disclosure comprises a compound of Formula (I):

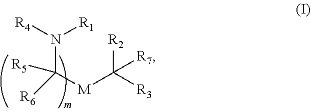

or a salt or isomer thereof, wherein:

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —O(CH₂)ₙOR, —N(R)C(═NR₉)N(R)₂, —N(R)C(═CHR₉)N(R₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

R₈ is selected from the group consisting of C₃₋₆ carbocycle and heterocycle;

R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄, alkenyl;

each R* is independently selected from the group consisting of C₁₋₁₂ alkyl and C₂₋₁₂ alkenyl;

each Y is independently a C3 carbocycle;

each X is independently selected from the group consisting of F, Cl, Br, and I; and

m is selected from 5, 6, 7, 8, 9, 10, 11, 12, and 13.

In some embodiments, a subset of compounds of Formula (I) includes those in which when R₄ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

In some embodiments, another subset of compounds of Formula (I) includes those in which

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₁₋₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and a 5- to 14-membered heterocycloalkyl having one or more heteroatoms selected from N, O, and S which is substituted with one or more substituents selected from oxo (═O), O, amino, mono- or di-alkylamino, and C₁₋₃ alkyl, and each n is independently selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁, alkyl, C₂₋₃ alkenyl, and H;

R₈ is selected from the group consisting of C₃₋₆ carbocycle and heterocycle;

R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13,

or salts or isomers thereof.

In some embodiments, another subset of compounds of Formula (I) includes those in which

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heterocycle having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5; and when Q is a 5- to 14-membered heterocycle and (i) R₄ is —(CH₂)ₙQ in which n is 1 or 2, or (ii) R₄ is —(CH₂)ₙCHQR in which n is 1, or (iii) R⁴ is —CHQR, and —CQ(R)₂, then Q is either a 5- to 14-membered heteroaryl or 8- to 14-membered heterocycloalkyl;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group:

R₇ is selected from the group consisting of C₁₋₃ alkyl, C₂₋₃ alkenyl, and H;

R₈ is selected from the group consisting of C₃₋₆ carbocycle and heterocycle;

R₉ is selected from the group consisting of H, CN, NO₂, C₁₋₆, alkyl, —OR, —S(O)₂R, —S(O)₂N(R)₂, C, alkenyl, C3.6 carbocycle and heterocycle;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13.

or salts or isomers thereof.

In some embodiments, another subset of compounds of Formula (I) includes those in which

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₄)N(R)₂, —C(═NR₉)R, —C(O)N(R)OR, and —C(═NR₉)N(R)₂, and each n is independently selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

R₈ is selected from the group consisting of C₃₋₆ carbocycle and heterocycle;

R₉ is selected from the group consisting of H, CN, NO₂, C₁, alkyl, —OR, —S(O)₂R, —S(O)₂N(R)₂, C₂₋₆ alkenyl, C₃₋₆, carbocycle and heterocycle;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13,

or salts or isomers thereof.

In some embodiments, another subset of compounds of Formula (I) includes those in which

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₂₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is —(CH₂)ₙQ or —(CH₂)ₙCHQR, where Q is —N(R)₂, and n is selected from 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

each R is independently selected from the group consisting of C₁₋₃ alkyl, C₂₋₃ alkenyl, and H:

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₁₋₁₂ alkenyl;

each Y is independently a C₃₋₆ carbocycle;

each X is independently selected from the group consisting of F, Cl. Br, and I; and

m is selected from 5, 6, 7, 8, 9, 10, 11, 12, and 13,

or salts or isomers thereof.

In some embodiments, another subset of compounds of Formula (I) includes those in which

R₁ is selected from the group consisting of C₅₋₃₀ alkyl, C₅₋₂₀ alkenyl, —R*YR″, —YR″, and —R″M′R′;

R₂ and R₃ are independently selected from the group consisting of C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of —(CH₂)ₙQ, —(CH₂)ₙCHQR, —CHQR, and —CQ(R)₂, where Q is —N(R)₂, and n is selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₁₋₁₂ alkenyl;

each Y is independently a C₃₋₆ carbocycle;

each X is independently selected from the group consisting of F. Cl, Br, and I; and

m is selected from 5, 6, 7, 8, 9, 10, 11, 12, and 13,

or salts or isomers thereof.

In some embodiments, a subset of compounds of Formula (I) includes those of Formula (IA):

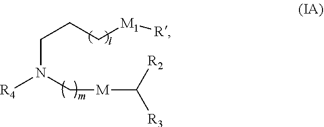

or a salt or isomer thereof, wherein 1 is selected from 1, 2, 3, 4, and 5; m is selected from 5, 6, 7, 8, and 9; M₁ is a bond or M′; R₄ is unsubstituted C₁₋₃ alkyl, or —(CH₂)ₙQ, in which Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, a subset of compounds of Formula (I) includes those of Formula (II):

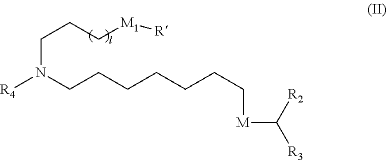

or a salt or isomer thereof, wherein 1 is selected from 1, 2, 3, 4, and 5; M, is a bond or M′; R₄ is unsubstituted C₁₋₃ alkyl, or —(CH₂)ₙQ, in which n is 2, 3, or 4, and Q is OH, —NHC(S)N(R)₂, —NHC(O)N(R)₂, —N(R)C(O)R, —N(R)S(O)₂R, —N(R)Rx, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, a subset of compounds of Formula (I) includes those of Formula (IIa), (IIb), (IIc), or (IIe):

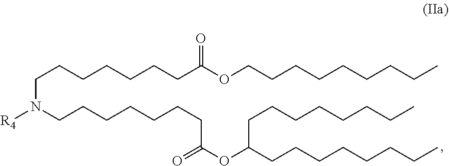

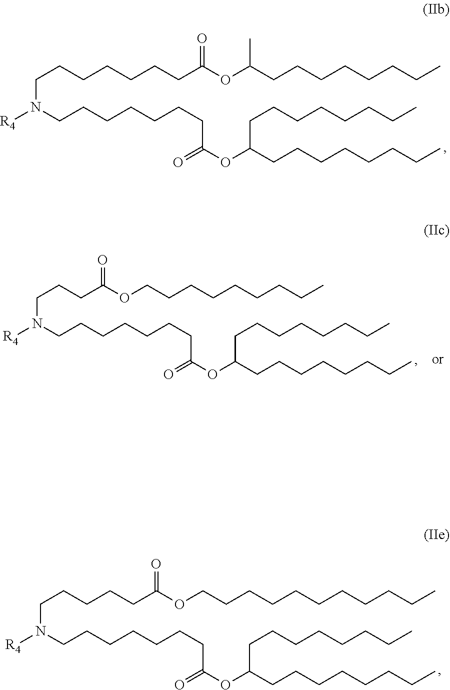

or a salt or isomer thereof, wherein R₄ is as described herein.

In some embodiments, a subset of compounds of Formula (I) includes those of Formula (IId):

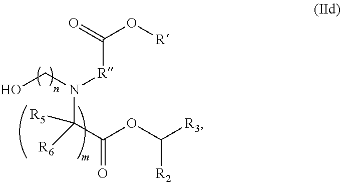

or a salt or isomer thereof, wherein n is 2, 3, or 4 and m, R′, R″, and R₂ through R₆ are as described herein. For example, each of R₂ and R₃ may be independently selected from the group consisting of C₅₋₁₄ alkyl and C₅₋₁₄ alkenyl.

In some embodiments, an ionizable cationic lipid of the disclosure comprises a compound having structure:

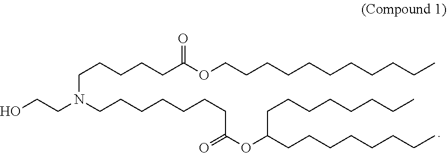 [SMILES: CCCCCCCCCCCOC(=O)CCCCCN(CCO)CCCCCCCC(=O)OC(CCCCCCCC)CCCCCCCC]

In some embodiments, an ionizable cationic lipid of the disclosure comprises a compound having structure:

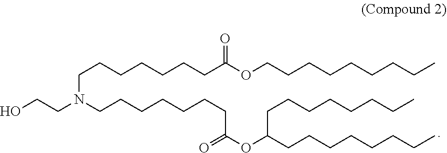 [SMILES: CCCCCCCCCOC(=O)CCCCCCCN(CCO)CCCCCCCC(=O)OC(CCCCCCCC)CCCCCCCC]

In some embodiments, a non-cationic lipid of the disclosure comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine,1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-dislearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In some embodiments, a PEG modified lipid of the disclosure comprises a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG and/or PEG-DPG.

In some embodiments, a sterol of the disclosure comprises cholesterol, fecosterol, sitosterol, ergosterol, campesterol, stigmasterol, brassicasterol, tomatidine, ursolic acid, alpha-tocopherol, and mixtures thereof.

In some embodiments, a LNP of the disclosure comprises an ionizable cationic lipid of Compound 1, wherein the non-cationic lipid is DSPC, the structural lipid that is cholesterol, and the PEG lipid is PEG-DMG.

In some embodiments, a LNP of the disclosure comprises an N:P ratio of from about 2:1 to about 30:1.

In some embodiments, a LNP of the disclosure comprises an N:P ratio of about 6:1.

In some embodiments, a LNP of the disclosure comprises an N:P ratio of about 3:1.

In some embodiments, a LNP of the disclosure comprises a wt/wt ratio of the ionizable cationic lipid component to the RNA of from about 10:1 to about 100:1.

In some embodiments, a LNP of the disclosure comprises a wt/wt ratio of the ionizable cationic lipid component to the RNA of about 20:1.

In some embodiments, a LNP of the disclosure comprises a wt/wt ratio of the ionizable cationic lipid component to the RNA of about 10:1.

In some embodiments, a LNP of the disclosure has a mean diameter from about 50 nm to about 150 nm.

In some embodiments, a LNP of the disclosure has a mean diameter from about 70 nm to about 120 nm.

### Preparation of High Purity RNA

In order to enhance the purity of synthetically produced RNA, modified in vitro transcription (IVT) processes which produce RNA preparations having vastly different properties from RNA produced using a traditional IVT process may be used. The RNA preparations produced according to these methods have properties that enable the production of qualitatively and quantitatively superior compositions. Even when coupled with extensive purification processes, RNA produced using traditional IVT methods is qualitatively and quantitatively distinct from the RNA preparations produced by the modified IVT processes. For instance, the purified RNA preparations are less immunogenic in comparison to RNA preparations made using traditional IVT. Additionally, increased protein expression levels with higher purity are produced from the purified RNA preparations.

Traditional IVT reactions are performed by incubating a DNA template with an RNA polymerase and equimolar quantities of nucleotide triphosphates, including GTP, ATP, CTP, and UTP in a transcription buffer. An RNA transcript having a 5′ terminal guanosine triphosphate is produced from this reaction. These reactions also result in the production of a number of impurities such as double stranded and single stranded RNAs which are immunostimulatory and may have an additive impact. The purity methods described herein prevent formation of reverse complements and thus prevent the innate immune recognition of both species. In some embodiments the modified IVT methods result in the production of RNA having significantly reduced T cell activity than an RNA preparation made using prior art methods with equimolar NTPs. The prior art attempts to remove these undesirable components using a series of subsequent purification steps. Such purification methods are undesirable because they involve additional time and resources and also result in the incorporation of residual organic solvents in the final product, which is undesirable for a pharmaceutical product. It is labor and capital intensive to scale up processes like reverse phase chromatography (RP): utilizing for instance explosion proof facilities. HPLC columns and purification systems rated for high pressure, high temperature, flammable solvents etc. The scale and throughput for large scale manufacture are limited by these factors. Subsequent purification is also required to remove alkylammonium ion pair utilized in RP process. In contrast the methods described herein even enhance currently utilized methods (e.g. RP). Lower impurity load leads to higher purification recovery of full length RNA devoid of cytokine inducing contaminants e.g. higher quality of materials at the outset.

The modified IVT methods involve the manipulation of one or more of the reaction parameters in the TVT reaction to produce a RNA preparation of highly functional RNA without one or more of the undesirable contaminants produced using the prior art processes. One parameter in the IVT reaction that may be manipulated is the relative amount of a nucleotide or nucleotide analog in comparison to one or more other nucleotides or nucleotide analogs in the reaction mixture (e.g., disparate nucleotide amounts or concentration). For instance, the IVT reaction may include an excess of a nucleotides, e.g., nucleotide monophosphate, nucleotide diphosphate or nucleotide triphosphate and/or an excess of nucleotide analogs and/or nucleoside analogs. The methods produce a high yield product which is significantly more pure than products produced by traditional IVT methods.

Nucleotide analogs are compounds that have the general structure of a nucleotide or are structurally similar to a nucleotide or portion thereof. In particular, nucleotide analogs are nucleotides which contain, for example, an analogue of the nucleic acid portion, sugar portion and/or phosphate groups of the nucleotide. Nucleotides include, for instance, nucleotide monophosphates, nucleotide diphosphates, and nucleotide triphosphates. A nucleotide analog, as used herein is structurally similar to a nucleotide or portion thereof but does not have the typical nucleotide structure (nucleobase-ribose-phosphate). Nucleoside analogs are compounds that have the general structure of a nucleoside or are structurally similar to a nucleoside or portion thereof. In particular, nucleoside analogs are nucleosides which contain, for example, an analogue of the nucleic acid and/or sugar portion of the nucleoside.

The nucleotide analogs useful in the methods are structurally similar to nucleotides or portions thereof but, for example, are not polymerizable by T7. Nucleotide/nucleoside analogs as used herein (including C, T, A, U, G, dC, dT, dA, dU, or dG analogs) include for instance, antiviral nucleotide analogs, phosphate analogs (soluble or immobilized, hydrolyzable or non-hydrolyzable), dinucleotide, trinucleotide, tetranucleotide, e.g., a cap analog, or a precursor/substrate for enzymatic capping (vaccinia, or ligase), a nucleotide labelled with a functional group to facilitate ligation/conjugation of cap or 5′ moiety (IRES), a nucleotide labelled with a 5′ PO4 to facilitate ligation of cap or 5′ moiety, or a nucleotide labelled with a functional group/protecting group that can be chemically or enzymatically cleavable. Antiviral nucleotide/nucleoside analogs include but are not limited to Ganciclovir, Entecavir, Telbivudine. Vidarabine and Cidofovir.

The IVT reaction typically includes the following: an RNA polymerase. e.g., a T7 RNA polymerase at a final concentration of, e.g., 1000-12000 U/mL, e.g., 7000 U/mL; the DNA template at a final concentration of, e.g., 10-70 nM, e.g., 40 nM; nucleotides (NTPs) at a final concentration of e.g., 0.5-10 mM, e.g., 7.5 mM each; magnesium at a final concentration of, e.g., 12-60 mM, e.g., magnesium acetate at 40 mM; a buffer such as, e.g., HEPES or Tris at a pH of, e.g., 7-8.5. e.g. 40 mM Tris HCl. pH 8. In some embodiments 5 mM dithiothreitol (DTT) and/or 1 mM spermidine may be included. In some embodiments, an RNase inhibitor is included in the IVT reaction to ensure no RNase induced degradation during the transcription reaction. For example, murine RNasc inhibitor can be utilized at a final concentration of 1000 U/mL. In some embodiments a pyrophosphatase is included in the IVT reaction to cleave the inorganic pyrophosphate generated following each nucleotide incorporation into two units of inorganic phosphate. This ensures that magnesium remains in solution and does not precipitate as magnesium pyrophosphate. For example, an E. coli inorganic pyrophosphatase can be utilized at a final concentration of 1 U/mL.

Similar to traditional methods, the modified method may also be produced by forming a reaction mixture comprising a DNA template, and one or more NTPs such as ATP, CTP, UTP, GTP (or corresponding analog of aforementioned components) and a buffer. The reaction is then incubated under conditions such that the RNA is transcribed. However, the modified methods utilize the presence of an excess amount of one or more nucleotide, and/or nucleotide analogs that can have significant impact on the end product. These methods involve a modification in the amount (e.g., molar amount or quantity) of nucleotides and/or nucleotide analogs in the reaction mixture. In some aspects, one or more nucleotides and/or one or more nucleotide analogs may be added in excess to the reaction mixture. An excess of nucleotides and/or nucleotide analogs is any amount greater than the amount of one or more of the other nucleotides such as NTPs in the reaction mixture. For instance, an excess of a nucleotide and/or nucleotide analog may be a greater amount than the amount of each or at least one of the other individual NTPs in the reaction mixture or may refer to an amount greater than equimolar amounts of the other NTPs.

In the embodiment when the nucleotide and/or nucleotide analog that is included in the reaction mixture is an NTP, the NTP may be present in a higher concentration than all three of the other NTPs included in the reaction mixture. The other three NTPs may be in an equimolar concentration to one another. Alternatively one or more of the three other NTPs may be in a different concentration than one or more of the other NTPs.

Thus, in some embodiments the IVT reaction may include an equimolar amount of nucleotide triphosphate relative to at least one of the other nucleotide triphosphates.

In some embodiments the RNA is produced by a process or is preparable by a process comprising

(a) forming a reaction mixture comprising a DNA template and NTPs including adenosine triphosphate (ATP), cytidine triphosphate (CTP), uridine triphosphate (UTP), guanosine triphosphate (GTP) and optionally guanosine diphosphate (GDP), and (e.g. buffer containing T7 co-factor e.g. magnesium).

(b) incubating the reaction mixture under conditions such that the RNA is transcribed, wherein the concentration of at least one of GTP, CFP, ATP, and UTP is at least 2× greater than the concentration of any one or more of ATP, CTP or UTP or the reaction further comprises a nucleotide analog and wherein the concentration of the nucleotide analog is at least 2× greater than the concentration of any one or more of ATP, CTP or UTP.

In some embodiments the ratio of concentration of GTP to the concentration of any one ATP, CTP or UTP is at least 2:1, at least 3:1, at least 4:1, at least 5:1 or at least 6:1. The ratio of concentration of GTP to concentration of ATP, CTP and UTP is, in some embodiments 2:1, 4:1 and 4:1, respectively. In other embodiments the ratio of concentration of GTP to concentration of ATP, CTP and UTP is 3:1, 6:1 and 6:1, respectively. The reaction mixture may comprise GTP and GDP and wherein the ratio of concentration of GTP plus GDP to the concentration of any one of ATP, CTP or UTP is at least 2:1, at least 3:1, at least 4:1, at least 5:1 or at least 6:1 In some embodiments the ratio of concentration of GTP plus GDP to concentration of ATP, CTP and UTP is 3:1, 6:1 and 6:1, respectively.

In some embodiments the method involves incubating the reaction mixture under conditions such that the RNA is transcribed, wherein the effective concentration of phosphate in the reaction is at least 150 mM phosphate, at least 160 mM, at least 170 mM, at least 180 mM, at least 190 mM, at least 200 mM, at least 210 mM or at least 220 mM. The effective concentration of phosphate in the reaction may be 180 mM. The effective concentration of phosphate in the reaction in some embodiments is 195 mM. In other embodiments the effective concentration of phosphate in the reaction is 225 mM.

In other embodiments the RNA is produced by a process or is preparable by a process comprising wherein a buffer magnesium-containing buffer is used when forming the reaction mixture comprising a DNA template and ATP, CTP, UTP, GTP. In some embodiments the magnesium-containing buffer comprises Mg2+ and wherein the molar ratio of concentration of ATP plus CTP plus UTP pus GTP to concentration of Mg2+ is at least 1.0, at least 1.25, at least 1.5, at least 1.75, at least 1.85, at least 3 or higher. The molar ratio of concentration of ATP plus CTP plus UTP pus GTP to concentration of Mg2+ may be 1.5. The molar ratio of concentration of ATP plus CTP plus UTP pus GTP to concentration of Mg2+ in some embodiments is 1.88. The molar ratio of concentration of ATP plus CTP plus UTP pus GTP to concentration of Mg2+ in some embodiments is 3.

In some embodiments the composition is produced by a process which does not comprise an dsRNase (e.g., RNaseIII) treatment step. In other embodiments the composition is produced by a process which does not comprise a reverse phase (RP) chromatography purification step. In yet other embodiments the composition is produced by a process which does not comprise a high-performance liquid chromatography (HPLC) purification step.

In some embodiments the ratio of concentration of GTP to the concentration of any one ATP, CTP or UTP is at least 2:1, at least 3:1, at least 4:1, at least 5:1 or at least 6:1 to produce the RNA.

The purity of the products may be assessed using known analytical methods and assays. For instance, the amount of reverse complement transcription product or cytokine-inducing RNA contaminant may be determined by high-performance liquid chromatography (such as reverse-phase chromatography, size-exclusion chromatography), Bioanalyzer chip-based electrophoresis system. ELISA, flow cytometry, acrylamide gel, a reconstitution or surrogate type assay. The assays may be performed with or without nuclease treatment (PI, RNase II, RNase H etc.) of the RNA preparation. Electrophoretic/chromatographic/mass spec analysis of nuclease digestion products may also be performed.

In some embodiments the purified RNA preparations comprise contaminant transcripts that have a length less than a full length transcript, such as for instance at least 100, 200, 300, 400, 500, 600, 700, 800, or 900 nucleotides less than the full length. Contaminant transcripts can include reverse or forward transcription products (transcripts) that have a length less than a full length transcript, such as for instance at least 100, 200, 300, 400, 500, 600, 700, 800, or 900 nucleotides less than the full length. Exemplary forward transcripts include, for instance, abortive transcripts. In certain embodiments the composition comprises a tri-phosphate poly-U reverse complement of less than 30 nucleotides. In some embodiments the composition comprises a tri-phosphate poly-U reverse complement of any length hybridized to a full length transcript. In other embodiments the composition comprises a single stranded tri-phosphate forward transcript. In other embodiments the composition comprises a single stranded RNA having a terminal tri-phosphate-G. In other embodiments the composition comprises single or double stranded RNA of less than 12 nucleotides or base pairs (including forward or reverse complement transcripts). In any of these embodiments the composition may include less than 50%, 45%, 40%, 35%, 30%, 25%, 20%, 15%, 10%, 9%, 8%, 7%, 6%, 5%, 4%, 3%, 2%, 1%, or 0.5% of any one of or combination of these less than full length transcripts.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having.” “containing,” “involving.” and variations thereof herein, is meant to encompass the items listed thereafter and equivalents thereof as well as additional items.

### EXAMPLES

### Example 1: Manufacture of Polynucleotides

According to the present disclosure, the manufacture of polynucleotides and or parts or regions thereof may be accomplished utilizing the methods taught in International Application WO2014/152027 entitled “Manufacturing Methods for Production of RNA Transcripts”, the contents of which is incorporated herein by reference in its entirety.

Purification methods may include those taught in International Application WO2014/152030 and WO2014/152031, each of which is incorporated herein by reference in its entirety.

Detection and characterization methods of the polynucleotides may be performed as taught in WO2014/144039, which is incorporated herein by reference in its entirety.

Characterization of the polynucleotides of the disclosure may be accomplished using a procedure selected from the group consisting of polynucleotide mapping, reverse transcriptase sequencing, charge distribution analysis, and detection of RNA impurities, wherein characterizing comprises determining the RNA transcript sequence, determining the purity of the RNA transcript, or determining the charge heterogeneity of the RNA transcript. Such methods are taught in, for example, WO2014/144711 and WO2014/144767, the contents of each of which is incorporated herein by reference in its entirety.

### Example 2: Chimeric Polynucleotide Synthesis

### Introduction

According to the present disclosure, two regions or parts of a chimeric polynucleotide may be joined or ligated using triphosphate chemistry.

According to this method, a first region or part of 100 nucleotides or less is chemically synthesized with a 5′ monophosphate and terminal 3′desOH or blocked OH. If the region is longer than 80 nucleotides, it may be synthesized as two strands for ligation.

If the first region or part is synthesized as a non-positionally modified region or part using in vitro transcription (IVT), conversion the 5′monophosphate with subsequent capping of the 3′ terminus may follow.

Monophosphate protecting groups may be selected from any of those known in the art.

The second region or part of the chimeric polynucleotide may be synthesized using either chemical synthesis or IVT methods. IVT methods may include an RNA polymerase that can utilize a primer with a modified cap. Alternatively, a cap of up to 130 nucleotides may be chemically synthesized and coupled to the IVT region or part.

It is noted that for ligation methods, ligation with DNA T4 ligase, followed by treatment with DNAse should readily avoid concatenation.

The entire chimeric polynucleotide need not be manufactured with a phosphate-sugar backbone. If one of the regions or parts encodes a polypeptide, then it is preferable that such region or part comprise a phosphate-sugar backbone.

Ligation is then performed using any known click chemistry, orthoclick chemistry, solulink, or other bioconjugate chemistries known to those in the art.

### Synthetic Route

The chimeric polynucleotide is made using a series of starting segments. Such segments include:

(a) Capped and protected 5′ segment comprising a normal 3′OH (SEG. 1)

(b) 5′ triphosphate segment which may include the coding region of a polypeptide and comprising a normal 3′OH (SEG. 2)

(c) 5′ monophosphate segment for the 3′ end of the chimeric polynucleotide (e.g., the tail) comprising cordycepin or no 3′OH (SEG. 3)

After synthesis (chemical or IVT), segment 3 (SEG. 3) is treated with cordycepin and then with pyrophosphatase to create the 5′monophosphate.

Segment 2 (SEG. 2) is then ligated to SEG. 3 using RNA ligase. The ligated polynucleotide is then purified and treated with pyrophosphatase to cleave the diphosphate. The treated SEG.2-SEG. 3 construct is then purified and SEG. 1 is ligated to the 5′ terminus. A further purification step of the chimeric polynucleotide may be performed.

Where the chimeric polynucleotide encodes a polypeptide, the ligated or joined segments may be represented as: 5′UTR (SEG. 1), open reading frame or ORF (SEG. 2) and 3′UTR+PolyA (SEG. 3).

The yields of each step may be as much as 90-95%.

### Example 3: PCR for cDNA Production

PCR procedures for the preparation of cDNA are performed using 2×KAPA HIFI™ HotStart ReadyMix by Kapa Biosystems (Woburn. Mass.). This system includes 2×KAPA ReadyMix12.5 μl; Forward Primer (10 μM) 0.75 μl; Reverse Primer (10 μM) 0.75 μl; Template cDNA −100 ng; and dH₂O diluted to 25.0 μl. The reaction conditions are at 95° C. for 5 min, and 25 cycles of 98° C. for 20 sec, then 58° C. for 15 sec, then 72° C. for 45 sec. then 72° C. for 5 min, then 4° C. to termination.

The reaction is cleaned up using Invitrogen's PURELINK™ PCR Micro Kit (Carlsbad. Calif.) per manufacturer's instructions (up to 5 μg). Larger reactions will require a cleanup using a product with a larger capacity. Following the cleanup, the cDNA is quantified using the NANODROP™ and analyzed by agarose gel electrophoresis to confirm the cDNA is the expected size. The cDNA is then submitted for sequencing analysis before proceeding to the in vitro transcription reaction.

### Example 4: In Vitro Transcription (IVT)

The in vitro transcription reaction generates polynucleotides containing uniformly modified polynucleotides. Such uniformly modified polynucleotides may comprise a region or part of the polynucleotides of the disclosure. The input nucleotide triphosphate (NTP) mix is made in-house using natural and un-natural NTPs.

A typical in vitro transcription reaction includes the following:
1 Template cDNA 1.0 μg
2 10× transcription buffer (400 mM Tris-HCl pH 8.0, 190 mM MgCl₂, 50 mM DTT, 10 mM Spermidine) 2.0 μl
3 Custom NTPs (25 mM each) 7.2 μl
4 RNase Inhibitor 20 U
5 T7 RNA polymerase 3000 U
6 dH₂0 Up to 20.0 μl, and
7 Incubation at 37° C. for 3 hr-5 hrs.

The crude IVT mix may be stored at 4° C. overnight for cleanup the next day. 1 U of RNase-free DNase is then used to digest the original template. After 15 minutes of incubation at 37° C., the mRNA is purified using Ambion's MEGACLEAR™ Kit (Austin. Tex.) following the manufacturer's instructions. This kit can purify up to 500 μg of RNA. Following the cleanup, the RNA is quantified using the NanoDrop and analyzed by agarose gel electrophoresis to confirm the RNA is the proper size and that no degradation of the RNA has occurred.

### Example 5: Enzymatic Capping

Capping of a polynucleotide is performed as follows where the mixture includes: IVT RNA 60 μg-180 μg and dH₂0 up to 72 μl. The mixture is incubated at 65° C. for 5 minutes to denature RNA, and then is transferred immediately to ice.

The protocol then involves the mixing of 10× Capping Buffer (0.5 M Tris-HCl (pH 8.0), 60 mM KCl, 12.5 mM MgCl₂) (10.0 μl); 20 mM GTP (5.0 μl); 20 mM S-Adenosyl Methionine (2.5 μl); RNase Inhibitor (100 U); 2′-O-Methyltransferase (400U); Vaccinia capping enzyme (Guanylyl transferase) (40 U); dH₂0 (Up to 28 μl); and incubation at 37° C. for 30 minutes for 60 μg RNA or up to 2 hours for 180 μg of RNA.

The polynucleotide is then purified using Ambion's MEGACLEAR™ Kit (Austin, Tex.) following the manufacturer's instructions. Following the cleanup, the RNA is quantified using the NANODROP™ (ThermoFisher, Waltham, Mass.) and analyzed by agarose gel electrophoresis to confirm the RNA is the proper size and that no degradation of the RNA has occurred. The RNA product may also be sequenced by running a reverse-transcription-PCR to generate the cDNA for sequencing.

### Example 6: PolyA Tailing Reaction

Without a poly-T in the cDNA, a poly-A tailing reaction must be performed before cleaning the final product. This is done by mixing Capped IVT RNA (100 μl); RNase Inhibitor (20 U): 10× Tailing Buffer (0.5 M Tris-HCl (pH 8.0), 2.5 M NaCl, 100 mM MgCl₂)(12.0 μl); 20 mM ATP (6.0 μl): Poly-A Polymerase (20 L); dH₂O up to 123.5 μl and incubation at 37° C. for 30 min. If the poly-A tail is already in the transcript, then the tailing reaction may be skipped and proceed directly to cleanup with Ambion's MEGACLEAR™ kit (Austin, Tex.) (up to 500 μg). Poly-A Polymerase is preferably a recombinant enzyme expressed in yeast.

It should be understood that the processivity or integrity of the polyA tailing reaction may not always result in an exact size polyA tail. Hence polyA tails of approximately between 40-200 nucleotides, e.g., about 40, 50, 60, 70, 80, 90, 91, 92, 93, 94, 95, 96, 97, 98, 99, 100, 101, 102, 103, 104, 105, 106, 107, 108, 109, 110, 150-165, 155, 156, 157, 158, 159, 160, 161, 162, 163, 164 or 165 are within the scope of the invention.

### Example 7: Natural 5′ Caps and 5′ Cap Analogues

5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′) G [the ARCA cap];G(5′)ppp(5′)A; G(5′)ppp(5′)G; m7G(5′)ppp(5′)A; m7G(5′)ppp(5′)G (New England BioLabs, Ipswich. Mass.). 5′-capping of modified RNA may be completed post-transcriptionally using a Vaccinia Virus Capping Enzyme to generate the “Cap 0” structure: m7G(5′)ppp(5′)G (New England BioLabs, Ipswich, Mass.). Cap 1 structure may be generated using both Vaccinia Virus Capping Enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase. Enzymes are preferably derived from a recombinant source.

When transfected into mammalian cells, the modified mRNAs have a stability of between 12-18 hours or more than 18 hours. e.g., 24, 36, 48, 60, 72 or greater than 72 hours.

### Example 8: Capping Assays

A. Protein Expression Assay

Polynucleotides encoding a polypeptide, containing any of the caps taught herein can be transfected into cells at equal concentrations, 6, 12, 24 and 36 hours post-transfection the amount of protein secreted into the culture medium can be assayed by ELISA. Synthetic polynucleotides that secrete higher levels of protein into the medium would correspond to a synthetic polynucleotide with a higher translationally-competent Cap structure.

B. Purity Analysis Synthesis

Polynucleotides encoding a polypeptide, containing any of the caps taught herein can be compared for purity using denaturing Agarose-Urea gel electrophoresis or HPLC analysis. Polynucleotides with a single, consolidated band by electrophoresis correspond to the higher purity product compared to polynucleotides with multiple bands or streaking bands. Synthetic polynucleotides with a single HPLC peak would also correspond to a higher purity product. The capping reaction with a higher efficiency would provide a more pure polynucleotide population.

C. Cytokine Analysis

Polynucleotides encoding a polypeptide, containing any of the caps taught herein can be transfected into cells at multiple concentrations. 6, 12, 24 and 36 hours post-transfection the amount of pro-inflammatory cytokines such as TNF-alpha and IFN-beta secreted into the culture medium can be assayed by ELISA. Polynucleotides resulting in the secretion of higher levels of pro-inflammatory cytokines into the medium would correspond to a polynucleotides containing an immune-activating cap structure.

D. Capping Reaction Efficiency

Polynucleotides encoding a polypeptide, containing any of the caps taught herein can be analyzed for capping reaction efficiency by LC-MS after nuclease treatment. Nuclease treatment of capped polynucleotides would yield a mixture of free nucleotides and the capped 5′-5-triphosphate cap structure detectable by LC-MS. The amount of capped product on the LC-MS spectra can be expressed as a percent of total polynucleotide from the reaction and would correspond to capping reaction efficiency. The cap structure with higher capping reaction efficiency would have a higher amount of capped product by LC-MS.

### Example 9: Agarose Gel Electrophoresis of Modified RNA or RT PCR Products

Individual polynucleotides (200-400 ng in a 20 μl volume) or reverse transcribed PCR products (200-400 ng) are loaded into a well on a non-denaturing 1.2% Agarose E-Gel (Invitrogen, Carlsbad, Calif.) and run for 12-15 minutes according to the manufacturer protocol.

### Example 10: Nanodrop Modified RNA Quantification and UV Spectral Data

Modified polynucleotides in TE buffer (1 μl) are used for Nanodrop UV absorbance readings to quantitate the yield of each polynucleotide from a chemical synthesis or in vitro transcription reaction.

### Example 11: Formulation of Modified mRNA Using Lipidoids

Polynucleotides are formulated for in vitro experiments by mixing the polynucleotides with the lipidoid at a set ratio prior to addition to cells. In vivo formulation may require the addition of extra ingredients to facilitate circulation throughout the body. To test the ability of these lipidoids to form particles suitable for in vivo work, a standard formulation process used for siRNA-lipidoid formulations may used as a starting point. After formation of the particle, polynucleotide is added and allowed to integrate with the complex. The encapsulation efficiency is determined using a standard dye exclusion assays.

### Example 12: Modified Nucleotides that Stabilize Coding Region Structure Enhance Protein Expression

### RNA Sequence and Nucleotide Modifications Combine to Determine Protein Expression

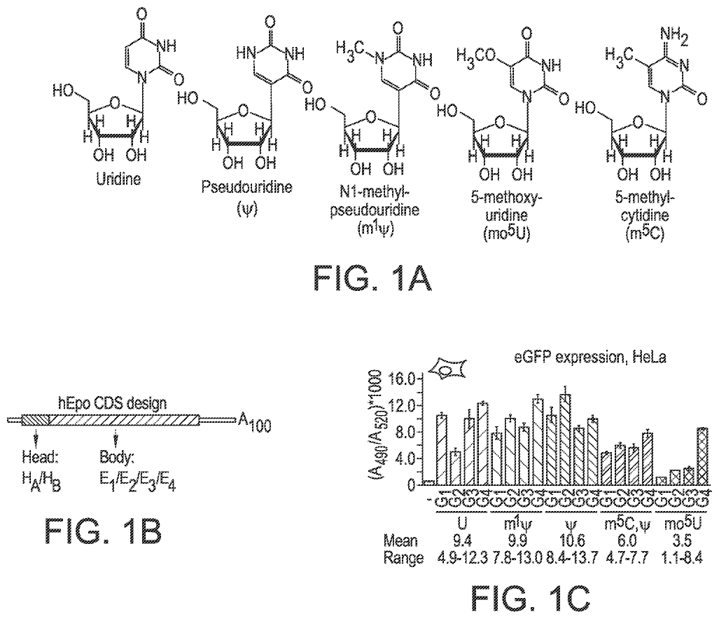

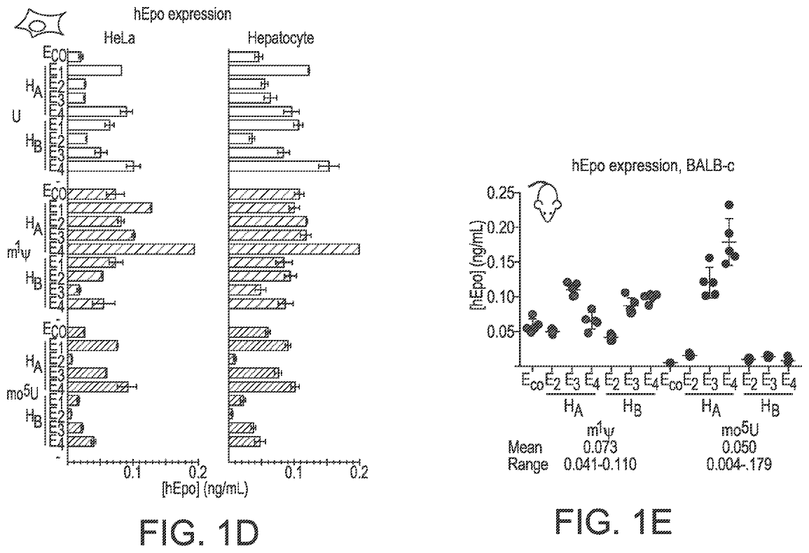

To probe the functional relationships between nucleotide modifications and primary RNA sequence, the effects of multiple base modifications in the context of a diverse set of synonymous CDS sequences encoding three different proteins: enhanced green fluorescent protein (eGFP; four variants), human erythropoietin (hEpo; nine variants) and firefly luciferase (Luc; thirty-nine variants) were studied. All mRNAs contained identical 5′ and 3′ UTRs, eGFP variants (G₁-G₄) were stochastically generated using only frequently used codons. For hEpo, one mammalian codon optimized sequence variant (E_{CO}) (Welch et al., 2009) was obtained, and eight variants were generated by combining two unique head sequences encoding the first 30 amino-acids (H_{A}, H_{B}) with four different variants of the remainder of the CDS (E₁, E₂, E₃, E₄) (FIG. 1B). A distinct, larger set of Luc variants deterministically encoded each amino acid with a single codon. All mRNAs were transcribed in vitro using either unmodified nucleotides or global substitutions of uridine (U) with the modified uridine analogs pseudouridine (Ψ), N′-methyl-pseudouridine (m¹Ψ), or 5-methyoxy-uridine (mo⁵U) (FIG. 1A). For eGFP. mRNA was also made substituting U and cytidine (C) with Ψ and 5-methyl-cytidine (m⁵C), respectively. These four modified nucleotides are known to reduce immunogenicity and therefore have direct application for therapeutic mRNAs (Andries et al., 2015; Kariko et al., 2008; Thess et al., 2015). All mRNAs carried a 7-methylguanylate cap (m⁷0-5′ppp5′-Gm) and a 100-nucleotide poly(A) tail.

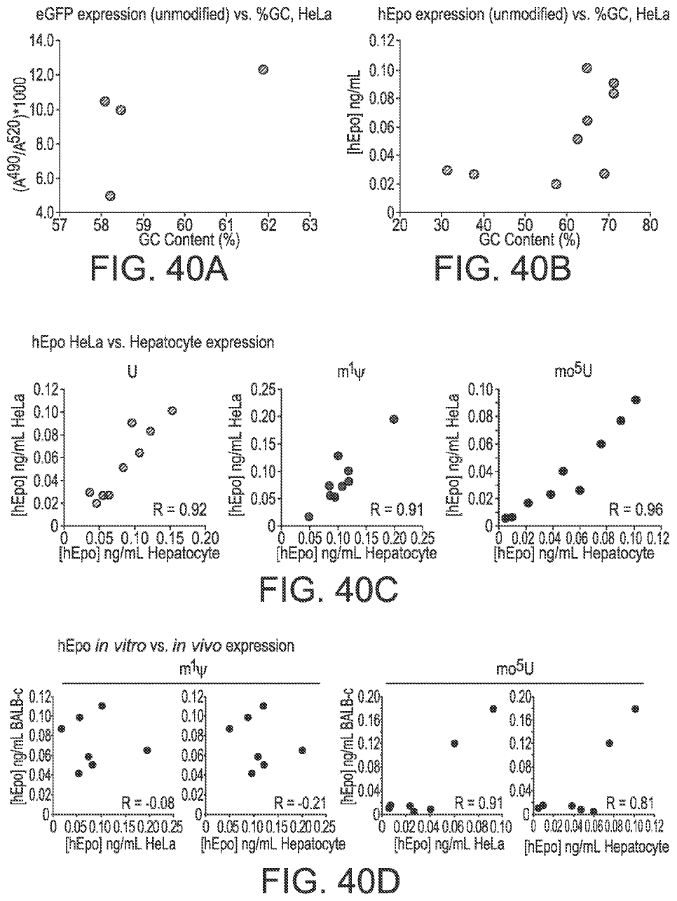

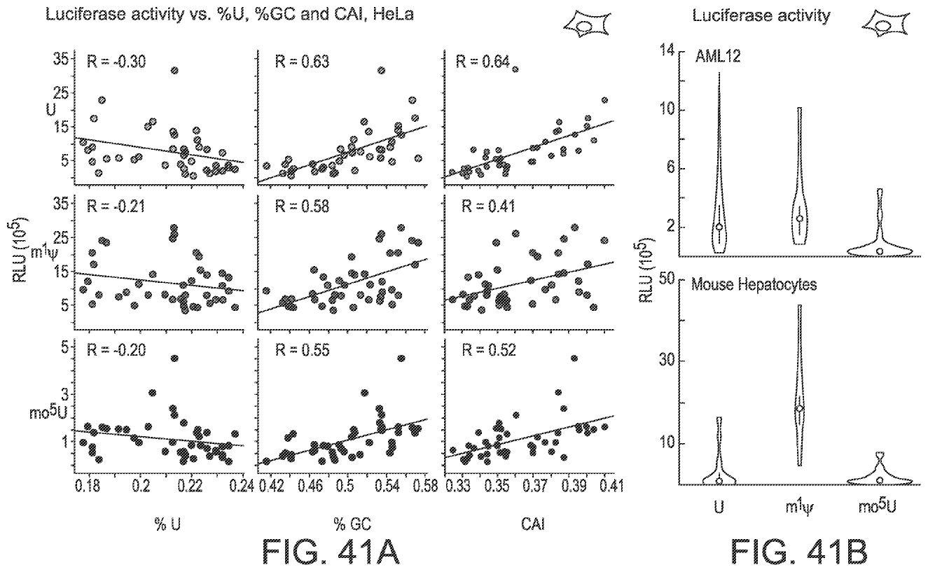

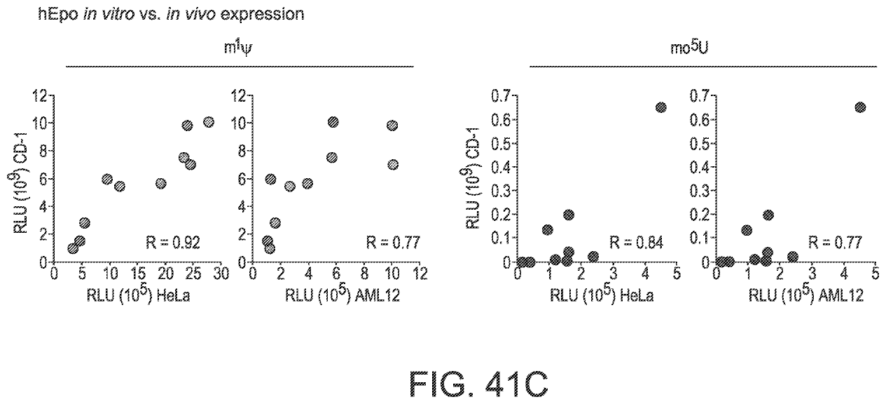

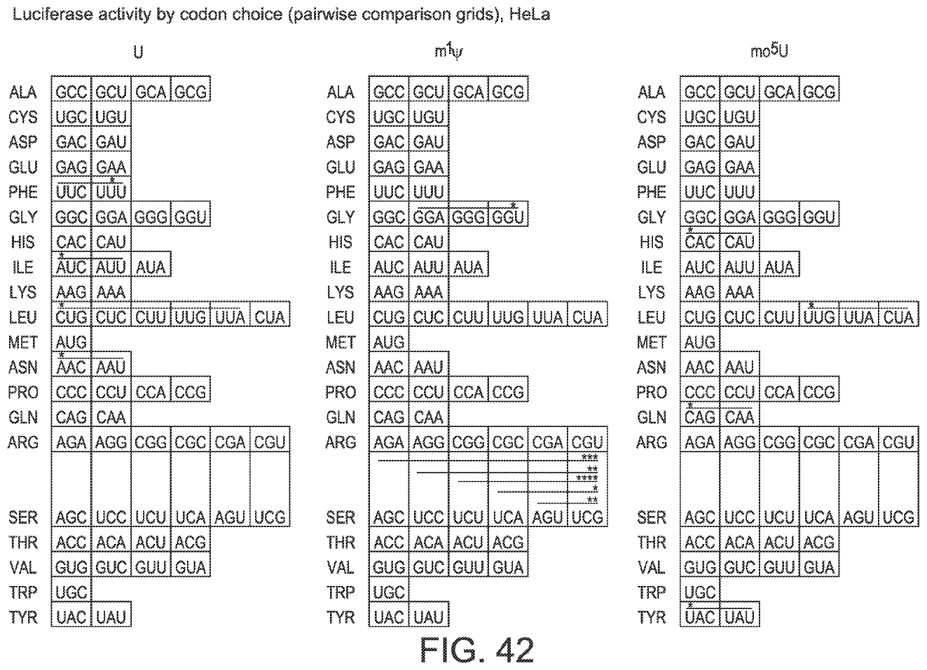

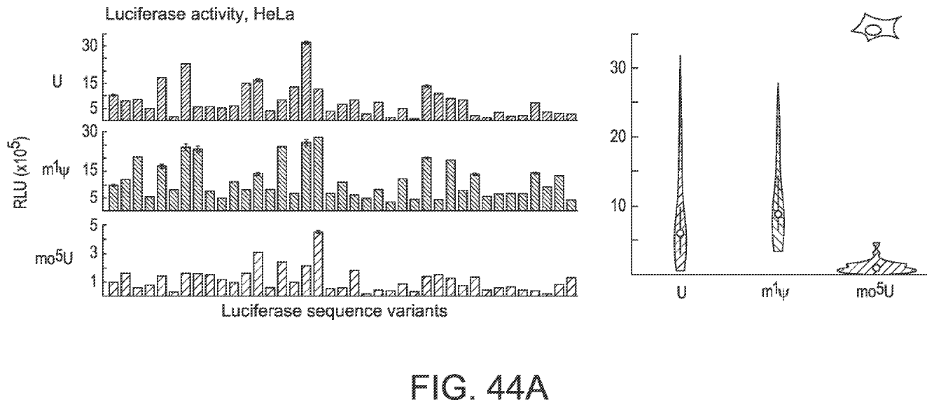

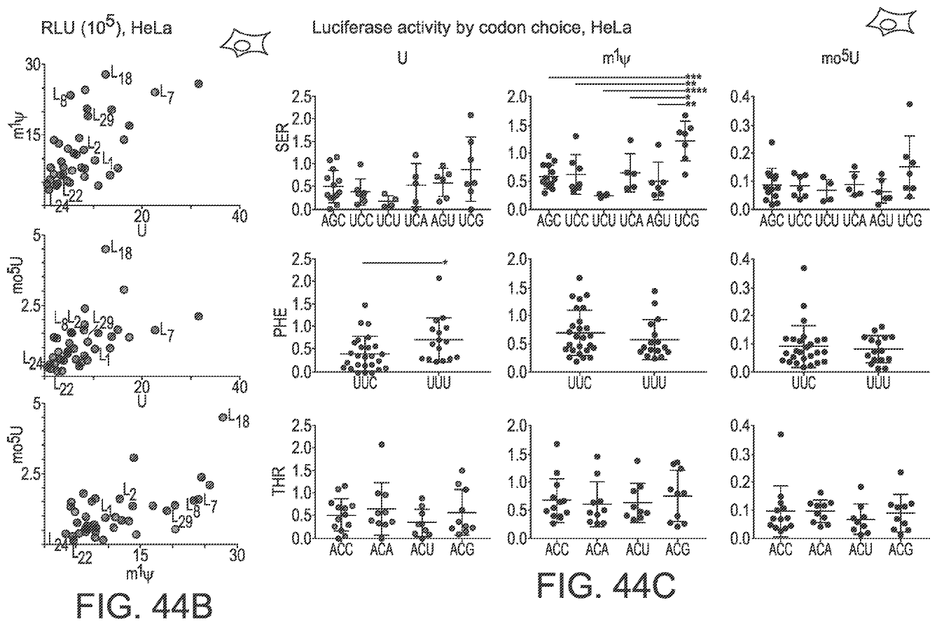

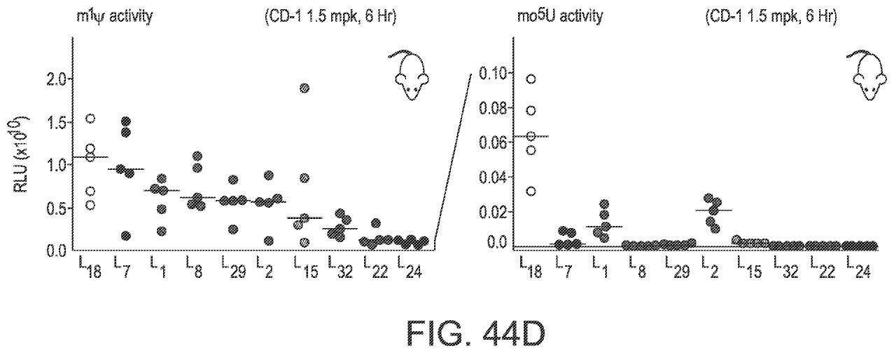

Consistent with previous reports (Gustafsson et al., 2004; Hinnebusch et al., 2016; Horstick et al., 2015; Pop et al., 2014), the CDS sequence were observed to greatly impact protein expression. Inclusion of modified nucleotides changed both the average level of protein expression and the range of expression caused by changes to the primary sequence to as measured by the ratio of the highest to lowest expressing mRNA. For mRNAs transcribed with unmodified nucleotides, cellular protein expression ranged >2.5-fold for eGFP (FIG. 1C, grey) and >4-fold for hEpo (FIG. 1D, grey), despite all sequences containing only frequent codons. For the 39 unmodified Luc variants expression ranged >10-fold (FIG. 44A). Consistent with previous reports (Plotkin and Kudla, 2011), highly expressed mRNAs tended to have increased GC content, but not all high GC CDSs were high expressers (FIGS. 40A, 40B, 41A, grey). For the 39 unmodified Luc variants using a greater diversity of codons, expression was moderately correlated with both GC-content and Codon Adaptation Index (CAI) (Pearson correlations 0.63 and 0.64, respectively. FIG. 41A, grey). The set of 39 unmodified Luc variants using only a single codon for each instance of a given amino acid allowed us to assess the impact of individual codons on protein expression. Only 4 out of a total of 87 pairwise comparisons between synonymous codons yielded statistical significant differences by ANOVA (p<0.05, FIG. 42, grey). For example, inclusion of codon Phe^{UUU} was associated with a slight increase in expression over Phe^{UUC} (FIG. 44C). Surprisingly, even consensus non-optimal codons, such as Ser^{UCG}, had negligible impacts on Luc expression in unmodified RNA (FIG. 44C) suggesting that multiple factors combine to regulate protein translation.

Next, the effect of global inclusion of different modified nucleotides on protein expression was examined. For eGFP encoding mRNAs, incorporation of modified nucleotides changed the expression of individual variants as well as the expression mean and range for the entire variant set. Compared to unmodified mRNA, the mean expression was slightly higher for Ψ and m¹Ψ mRNA. For mo⁵U and Ψ/m⁵C modified mRNAs; however, mean expression was 3-fold and 1.5-fold lower, respectively (FIG. 1C). Protein levels produced by unmodified RNA were relatively low, but this is likely to be caused by induction of the cells innate immune response, which was monitored by detection of secreted interferon beta in BJ fibroblasts. The relative sensitivity of the modified nucleotides to the RNA sequence was consistent with the previous results from eGFP mRNAs. Relative to unmodified mRNA, the number of poorly expressing eGFP variants decreased for Ψ and m¹Ψ mRNA but increased for mo⁵U containing mRNA. Of note, the identities of the best and worst expressing sequences changed with different modified nucleotides. For example, sequence G₂ yielded high expression in Ψ and m¹Ψ, poor expression in mo⁵U, and moderate expression in U and Ψ/m⁵C (FIG. 1C). Similar trends were observed for hEpo mRNA, with m¹Ψ yielding a 1.5-fold greater mean expression than U, which was 2-fold higher than mo⁵U (FIG. 1D). Again, hEpo variants (e.g., E_{CO} and H_{A}E₂ in HeLa) that expressed well with m¹Ψ mRNA but not U or mo⁵U-containing mRNA were observed (FIG. 1D). Although some variation in the expression of specific RNAs was observed, the general expression trends were highly similar in primary mouse hepatocytes (FIGS. 1D, 40C).

In order to confirm that protein expression levels observed in cell lines translate to expression in vivo, seven of the hEPO RNAs were formulated in two different chemistries (m¹Ψ and mo⁵U) in lipid nanoparticles (LNP) and delivered intravenously to BALB/C mice. Levels of circulating human EPO protein were assessed by ELISA 24 hours later. Similar to the results in cultured cells, levels of expressed protein were dependent upon both the primary sequence and the chemistry of the nucleotide used to encode the mRNA (FIG. 1D). The sensitivity of the modified mRNAs to the primary sequence was maintained in vivo, with mRNA containing m¹Ψ highly expressed across all sequence variants and mRNA containing mo⁵U hyper-sensitive to the primary sequence on the RNA. Consistent with the cell culture data, the codon optimized variant was highly expressed in the m¹Ψ RNA, but poorly expressed in the mo⁵U RNA, and the superior expression of m¹Ψ RNA in cell culture diminished in vivo. Importantly, protein expression from mo⁵U mRNA variants L1E2 and L1E3 matched or exceeded expression level of its respective counterpart in m¹It RNA. Further, the most potent hEpo mRNA was the L1E3 variant with mo⁵U which produced almost twice as much protein as the next best mRNA. These data illustrate the complex functional relationships between mRNA sequence and nucleotide chemistry in cells and in vivo.

To extend this analysis, 39 synonymous Luc sequences containing m¹Ψ or mo⁵U mRNA were examined in multiple cell lines. Compared to unmodified mRNA, the mean expression increased 1.5-fold for m¹Ψ mRNA but decreased 5-fold for mo⁵U (FIG. 44A). Although the distribution of protein expression from unmodified mRNA was consistently intermediate to m¹Ψ and mo⁵U mRNA across cell lines, it was closer to m¹Ψ mRNA in HeLa and AML12 cells but closer to mo⁵U mRNA in primary hepatocytes (FIGS. 44A, 41B). Relative protein expression from individual mRNA sequences harboring one modified nucleotide poorly predicted expression from mRNAs containing other nucleotides (FIG. 44B). For example, several sequences (e.g. L₂₄, and L₂₂) universally produced low levels of protein across all chemistries (FIG. 44B). However, many variants (e.g. L₁₈, L₇, L₂, L₈, and L₂₉) had differential relative expression that favored specific chemistries over others. Taken together, these data indicate that CDS sequence and nucleotide modifications make distinct contributions to determine the overall level of protein expression.

The expression differences observed could be simply explained by modified nucleotides directly influencing decoding. This model predicts that expression should correlate, either positively or negatively, with the total percent of modified nucleotides or alternatively, with inclusion or exclusion of specific codons with modified nucleotides. However, the total percentage of modified bases had no clear correlation with protein expression for any modified nucleotide (FIG. 41A). Additionally, only 10 out of 174 total pairwise comparisons between synonymous codons yielded statistically significant differences by ANOVA (p<0.05 (FIG. 42)). More specifically, use of codons containing modified uridines did not significantly impact protein expression, except for an unexpected increase in protein production with Ser^{UCG} in m¹Ψ mRNA (FIGS. 44C, 42). Thus, the modification-specific differences in protein expression observed were not due to the inclusion or avoidance of individual codons containing modified nucleotides.

Gene expression from an individual mRNA can vary both between cell lines and also between different tissues within the body. As the liver is one of the most bioavailable tissues for delivery of RNA therapeutics (Zhao. 2014), ten luciferase RNA variants were remade with the goal of testing in more clinically relevant experimental systems. AML12 and primary human hepatocytes. mRNAs representing a wide range of expression levels were selected from the original set of 42 and remade in both 5moU and 1mψ. Overall, the levels of expression with both of these cell lines correlated with the protein levels observed in HELA cells with the exception of some variability observed in moderately expressed.

This set of ten luciferase RNA were subsequently formulated in lipid-based nanoparticles (LNPs) and delivered the modified mRNAs by intravenous injection into CD-1 mice. Production of luciferase protein in vivo was measured at 6 hours, post-injection thorough whole animal imaging. As expected, the liver was the main site of protein expression for (FIG. 2B). Interestingly, the hyper-variability in protein expression observed in cell culture was exaggerated in the 5moU containing mRNA constructs. Luc76 mRNA was one of the few mRNAs that expressed luciferase protein, along with Luc5l and Luc52 to a much lesser amount (FIG. 2C). Seven of the ten sequence variants produced little if any RNA in the 5moU containing RNA. When combined with the previous data from eGFP and hEPO, these studies reveal that the chemical modification of RNA nucleotides in combination with the mRNA primary sequence determine the level of protein expression, and that protein expression from some modified nucleotides are hyper-sensitive to the primary sequence.

To compare protein expression in cell culture to protein expression in vivo, protein expression from formulated hEpo and Luc mRNA variants containing two nucleotide modifications with reduced immunogenicity (m¹IF and mo⁵U) was examined (Kariko et al., 2005). Unmodified mRNAs were excluded from the in vivo analysis because translational phenotypes are often obscured by strong activation of innate immunity. For some hEpo mRNAs, such as m¹Ψ H_{B}E₃, different levels of expression were observed between the cell lines and in vivo (FIG. 40D). These differences were larger than the differences observed between cell lines and more pronounced for m¹Ψ hEPO mRNA than for mo⁵U hEPO mRNA (FIG. 40D). They likely reflect differences in translation factors between the cell lines and the tissue. Moreover, the general trends like the sensitivity of the modified mRNAs to the primary sequence was maintained in vivo (FIGS. 1D, 1E). mRNAs containing m¹Ψ expressed well across all sequence variants (FIG. 1E). In contrast, mo⁵U mRNA expressed in only a few variants (FIG. 1E). The codon optimized variant E_{CO} expressed well with m¹Ψ but poorly in mo⁵U. Importantly, the best expressing RNAs in vivo were mo⁵U mRNA variants H_{A}E₄ and H_{A}E₃. The mo⁵U H_{A}E₄ mRNA produced almost twice as much protein as the second highest expressing variant (FIG. 1E).

Protein expression from ten Luc variants, selected because they exhibited a wide range of protein expression in cell culture, was tested in vivo. As expected (Kauffman et al., 2016), the liver was the main site of protein expression (FIG. 2B). mRNAs containing m¹Ψ were highly expressed in viva, particularly L₁₈ and L₇ (FIG. 44E, left panel). The variability in protein expression with mo⁵U was exaggerated in vivo as 7 of the 10 variants produced little to no protein (FIG. 44E, right panel). L₁₈ was an exception, but still produced >10-fold lower levels of Luc than the same sequence with m¹Ψ (FIG. 44E, right panel). Variants L₁ and L₂ with mo⁵U produced limited but detectable amounts of protein (FIG. 44E, right panel). Notably, L₇, which produced large amounts of protein with m¹Ψ produced barely detectable levels of protein with mo⁵U. These data suggest that expression differences observed in cell culture persist and can be more pronounced in the context of exogenous RNAs delivered in vivo (FIG. 41D).

Given the dramatic effect that chemical modification has on the relative amount of protein produced from a given mRNA sequence, the large set of 39 luciferase sequences were examined for primary sequence features that could explain chemistry-dependent expression differences. First, the total percentage of modified positions (U's) for both 1mψ and 5moU were examined and negligible correlations were found with expression (−0.02 and −0.24 respectively). Since the luciferase variants were designed using a single codon for each amino acid, whether use of any particular codon for each amino acid was associated with changes in protein expression was examined. A pair-wise comparison between synonymous codons failed to detect any changes in expression level based on the inclusion of individual codons that rose to the level of statistical significance (p<0.05). Notably, no expression defects in mRNAs containing modified nucleotides were observed when compared to synonymous codons containing unmodified nucleotides. This provides further confirmation that translational decoding is highly permissive of small modifications on the Hoogsteen edge of the nucleobase across all three codon positions. Combined, these functional expression data suggest that chemical modification of RNA impacts protein expression on a level that is distinct from that of the primary sequence. Therefore, the impact of modified nucleotides on the structural stability and secondary structure of mRNA were examined.

Protein Expression Differences Correlate with mRNA Thermodynamic Stability

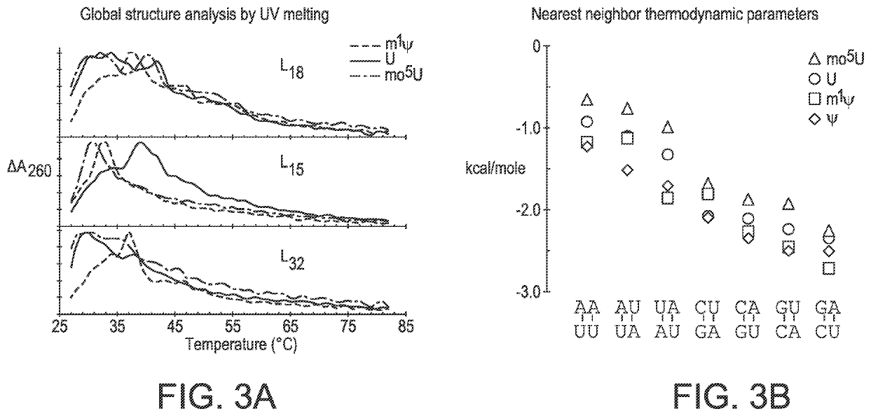

Analysis of the expression data suggested that modified nucleotides impact protein expression on a level above that of primary sequence. Therefore, how the modified nucleotides might affect mRNA structure was examined. Optical melting data was used to examine the structural stability of double-stranded features within three differentially expressed Luc mRNAs containing three different nucleotides (U, m¹Ψ, and mo⁵U). As the RNA is heated, the normalized first derivative of the UV-absorbance is a measure of the amount of RNA structure that melts at a given temperature. Two RNAs, Lis and L₃₂, had high and low relative expression respectively across all chemistries, and one RNA, L₁₅, expressed highly only in m¹Ψ. The highly expressing sequence variant (L₁₈) exhibits a major peak and multiple minor peaks between 35° C. to 65° C. in all chemistries tested (FIG. 3A, top panel). L₁₈ containing m¹Ψ, which expressed highly in vivo, had no peaks below 35° C. L₁₅ mRNA, which expressed poorly with mo⁵U but well with m¹Ψ, displayed a dramatic, modification-dependent shift in the UV-melting profile with only the m¹Ψ version having a major peak above 35° (FIG. 3A, middle panel). L₃₂ RNA, which expressed poorly across all nucleotides, had no major peak above 35° C. (FIG. 3A, bottom panel). Thus, the highly-expressed mRNA exhibited more secondary structure, in contrast to predictions that RNA structure would reduce translational efficiency (Gorochowski et al., 2015). These results provide a direct link between intrinsic RNA stability and modification-dependent protein expression in vivo.

Observations of global RNA structure were extended with optical melting experiments on 35 synthetic short RNA duplexes containing global substitutions of U with Ψ, m¹Ψ, and mo⁵U. The optical melting data for each set of modified duplexes were processed using established methodologies (Xia et al., 1998) to obtain the thermodynamic parameters for the nearest neighbor free energy of base pairing. Nearest neighbors containing Ψ (FIG. 3B, diamonds) and m¹Ψ (FIG. 3B, squares) are stabilized when compared to published values for uridine (FIG. 3B, circles; (Xia et al., 1998)) by 0.25 and 0.18 kcal/mol on average, respectively (FIG. 3B. Table 1). In contrast, nearest neighbors containing mo⁵U (FIG. 3B, triangles) are destabilized by 0.28 kcal/mol when compared to uridine (FIG. 3B, Table 1). For mo⁵U versus T, the differences average −0.5 kcal/mol per nearest neighbor, or −1.0 kcal/mol per base pair. The absolute energy differences between modified nucleotides deviates for some nearest neighbor pairs: for example, CU/GA is destabilized by both mo⁵U and Ψ compared to uracil (FIG. 3B) The cumulative differences from hundreds of base pairs containing modified nucleotides readily explain the global folding energy differences observed in the UV melting data and how sequence context defines the overall impact on structure. These data confirm that folding energy as determined by nucleotide modification inversely correlates with average protein expression.

TABLE 1
Nearest neighbor base pairing energies for modified nucleotides

| | Uridine | | | |
|---|---|---|---|---|
| Parameter | (Xia et al., 1998) | m¹Ψ | mo⁵U | Ψ |
| AA/UU | −0.93 | −1.18 | −0.66 | −1.23 |
| AU/UA | −1.1 | −1.13 | −0.77 | −1.52 |
| UA/AU | −1.33 | −1.86 | −1 | −1.71 |
| CU/GA | −2.08 | −1.8 | −1.69 | −2.1 |
| CA/GU | −2.11 | −2.27 | −1.88 | −2.35 |
| GU/CA | −2.24 | −2.46 | −1.93 | −2.5 |
| GA/CU | −2.35 | −2.72 | −2.26 | −2.51 |

Nearest-neighbor thermodynamic parameters for Watson-crick base pairs containing unmodified uridine (values from (Xia et al., 1998)), Ψ, m¹Ψ, or mo⁵U. The modified nucleotide(s) for each nearest neighbor pair is bolded. Parameters were derived by linear regression of UV-melting data from X short oligonucleotides containing global substitutions, as described in (Xia et al., 1998).

Modified Nucleotides Induce Global Rearrangement of mRNA Structure

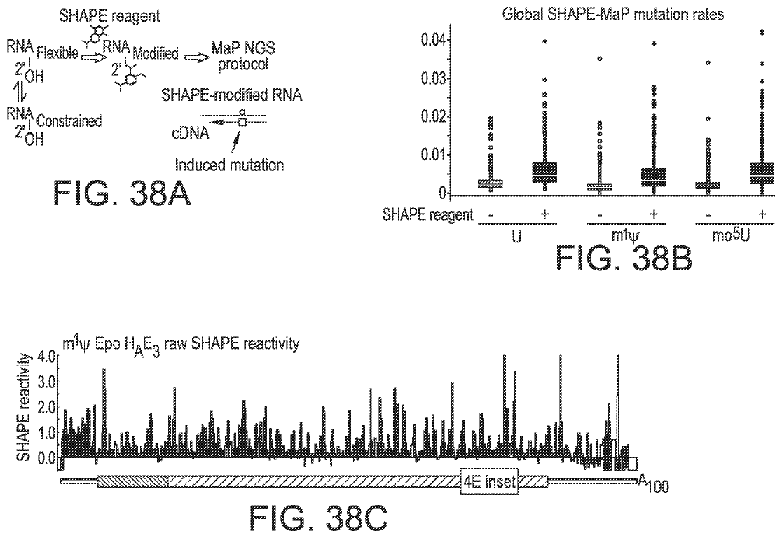

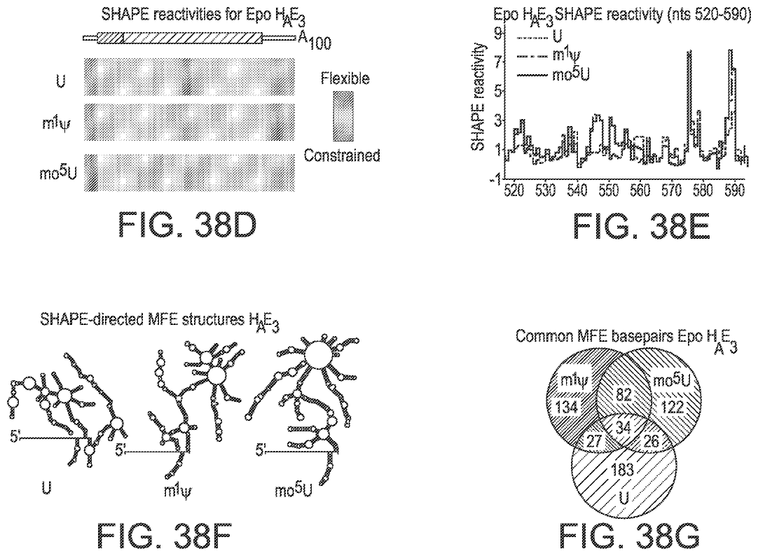

To investigate the mRNA structure-function relationships at single nucleotide resolution. SHAPE-MaP structure probing technology was used (Siegfried et al., 2014). SHAPE-MaP selectively modifies the RNA backbone with covalent adducts at the 2′ hydroxyl of flexible nucleotides. Adduct positions are subsequently detected by increases in mutation rate using Next-Generation Sequencing (FIG. 38A) (Smola et al., 2015). Detection of structural data using SHAPE depends on disruption to primer extension upon encountering a chemical adduct within the RNA. Since this is the first reported use of SHAPE on globally substituted m¹Ψ and mo⁵U RNAs, the methodology was validated first. There was no evidence of increased background NGS error rates for either m¹Ψ or mo⁵U RNA in the absence of SHAPE reagent, 1-methyl-6-nitroisatoic anhydride (FIG. 38B). Treatment with the SHAPE reagent uniformly increased the mutation rates across all RNA chemistries, consistent with previously reported values for this method (FIG. 38B) (Smola et al., 2015). It was concluded that SHAPE-MaP technology could be used effectively on globally modified mRNAs.

Using SHAPE-MaP, the presence of RNA structure across the experimentally tested variants of hEpo containing unmodified U, m¹Ψ, or moU nucleotides was measured. SHAPE-MaP produced single-nucleotide resolution structural information across the entire RNA, with stable structural elements indicated by low SHAPE reactivities (FIG. 38C). SHAPE data for hEpo mRNA H_{A}E₃ revealed modification-dependent, local structural differences across individual regions of the mRNA (FIGS. 38D, 38E). In many RNAs, such as hEpo H_{A}E₃, the mRNA flexibility as measured by SHAPE showed that m¹Ψ stabilized and mo⁵U destabilized structure (FIG. 38D), consistent with biophysical measurements described above. In addition to these global trends, regions where the flexibility of the bases changed greatly depending on the chemistry of the nucleotides but within the same sequence were observed (FIG. 3C), indicative of large-scale regional rearrangements in the structure. SHAPE reactivities values obtained from the chemically modified mRNAs were used as pseudo-free energy constraints to model RNA secondary structure utilizing a previously validated methodology to improve the accuracy of structural predictions (Deigan et al., 2009). The data-directed secondary structure models indicate that modified nucleotides induce wide-spread secondary structure rearrangements in many regions of the RNA (FIG. 38F). The minimum-free energy models of H_{A}E₃ predict that less than 13% of base pairs exist across all RNAs, and most predicted base pairs are unique to just one nucleotide chemistry (FIG. 38G). These findings indicate that incorporation of modified nucleotides induce widespread changes in the structural conformations of RNAs.

Postilion-Dependent Structural Context Defines Highly Expressed mRNAs

Using SHAPE-MaP, synonymous variants that displayed a range of expression phenotypes for of hEpo (8 variants with m¹Ψ and mo⁵U) and Luc (16 variants with m¹Ψ; 12 variants with moU) were characterized in order to establish a position-dependent functional relationship. Regions with structural differences were identified with median reactivities as previously described (Watts et al., 2009). Consistent with results described above, mRNA variants that were highly expressed in vivo had lower median SHAPE reactivities, indicating increased structure, across the CDS when compared to poorly expressing variants. This was true for both modified nucleotides and both proteins (FIGS. 4A, 4B). In mRNAs that expressed poorly specifically in mo⁵U, such as E_{CO} and L₈, a widespread increase in median SHAPE reactivity was observed, indicating disruption of structure, across the CDS only with mo⁵U (FIGS. 4A, 4B). In contrast to the CDS, the 5′ UTR was highly reactive across most variants tested, indicating that the common 5′ UTR was largely unstructured (FIGS. 4A-4B).

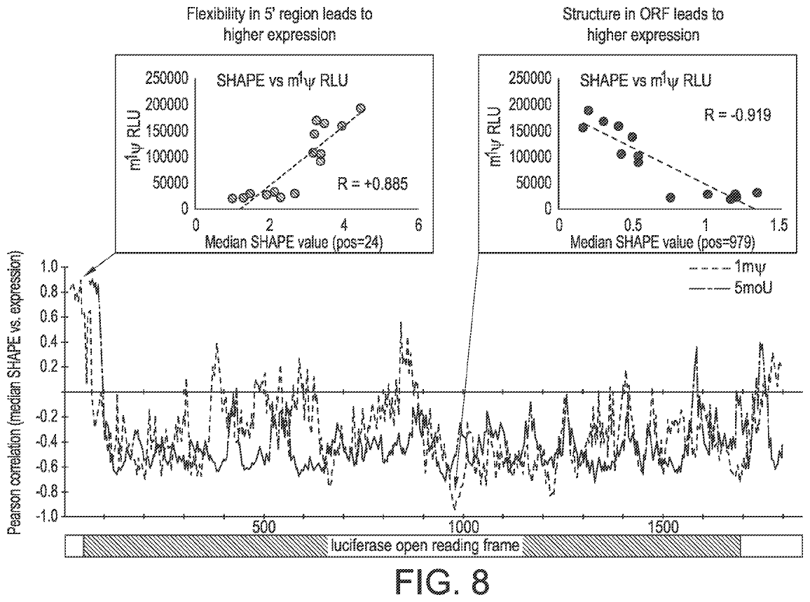

A Pearson correlation analysis was used to model and quantify the directionality and strength of the regional structure-function relationships across the Luc mRNA with m¹Ψ and mo⁵U (FIG. 8). The analysis revealed a striking, position-dependent structure-function relationship between mRNA structure and expression in HeLa cells that was consistent between mRNA with m¹Ψ and mo⁵U. A region encompassing the 47-nt 5′ UTR and the first −30 nucleotides of the CDS was defined by a very strong positive correlation (r≈0.8) between SHAPE reactivity and protein expression (FIG. 8, left inset). Flexibility within this first region strongly facilitated protein production, possibly through more efficient ribosome recruitment. This relationship dramatically inverted around nucleotide position 30 of the CDS to a moderate inverse correlation (r=≈0.6) for the remainder of the CDS and 3′ UTR with both m¹Ψ and mo⁵U (FIG. 8, right inset). When averaged over this second region, increased secondary structure correlated with improved protein expression, consistent with the global structural properties measured by optical melting. The strength of the structure-function correlation fluctuates across Luc mRNA, with strong negative correlations in specific regions, such as near position 950. Unexpectedly, the negative correlation between structure and protein expression was maintained near the stop codon (FIG. 8). However, the three sequential stop codons in these mRNAs likely enforce efficient termination. The observed structure-function correlations explain how structural changes induced by modified nucleotides could impact the protein expression of specific sequence variants.

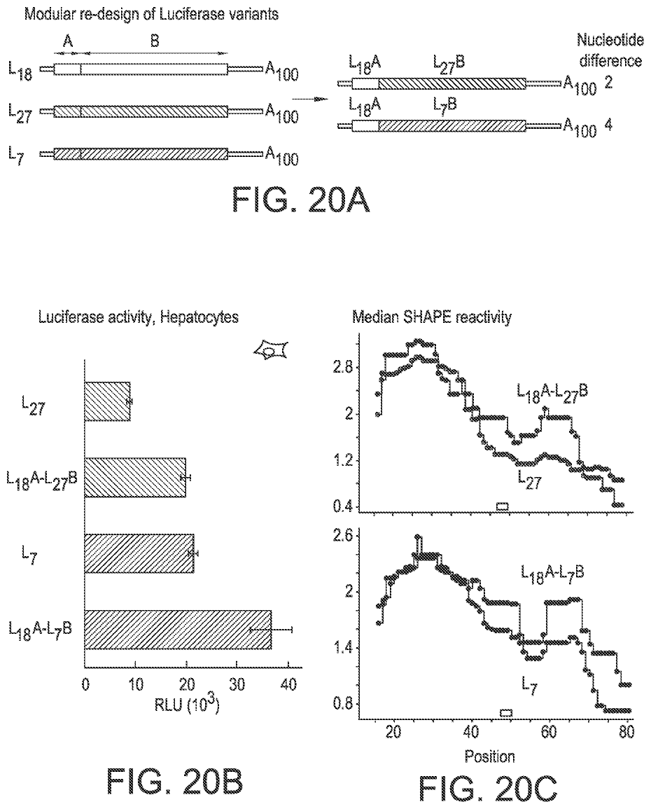

To test the importance of flexibility at the 5′ end, two m¹Ψ mRNAs with moderate expression, shown by SHAPE to contain similar degrees of structure within the CDS, but noticeably lower SHAPE reactivities (L₇ and L₁₇) around the start codon were selected. Chimeric sequences that combined the first 30 nucleotides of the L₁₈ variant containing flexible RNA around the start codon with the rest of the CDS from variants L₇ and L₂₇ (FIG. 20A) were designed. Both chimeric RNAs (L₁₈L₇ and L₁₈L₂₇) were shown by SHAPE to have increased RNA flexibility within region 1 (FIG. 20C). The chimera L₁₈L₇, which changed only two individual nucleotides relative to L₇, increased expression 1.5-fold, and chimera L₁₈L₂₇, which changed only four nucleotides, increased expression 2-fold (FIG. 20B). These data confirm that mRNAs that satisfy the two-part structural context described above express highly.

Structured mRNAs Primarily Impact Ribosome Association Rather than mRNA Half-Life

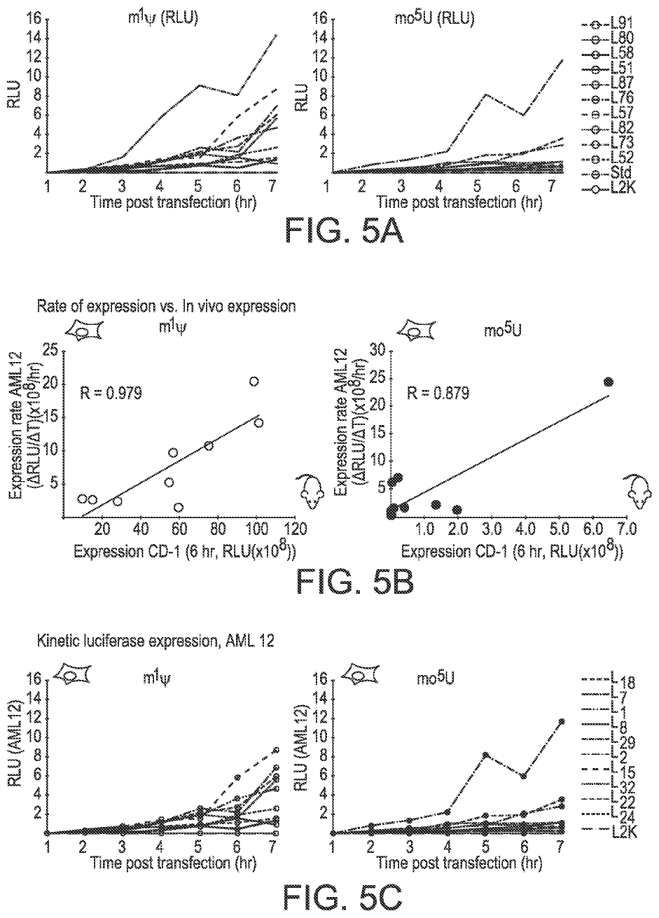

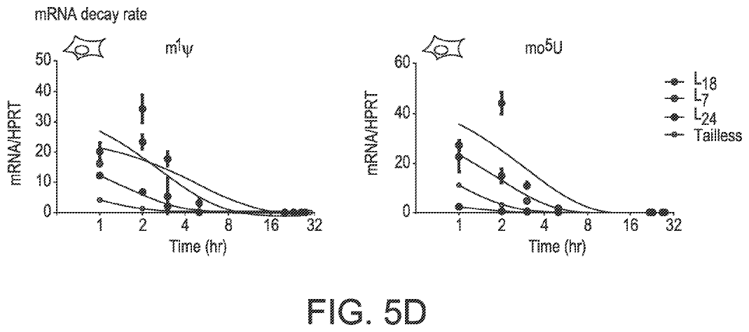

To investigate the causes of the above expression differences, the kinetics of both protein production and RNA degradation were examined across Luc variants. Eleven differentially expressed Luc mRNAs containing m¹Ψ or mo⁵U were transfected into AML12 cells and assayed for protein expression every hour for seven hours. Protein production occurred through the first 7 hours and by 24 hours the RNA had been degraded (FIGS. 5C, 5D). The average rate of protein expression through seven hours for mRNA variants in AML12 cells strongly correlated with protein expression in CD-1 mice in vivo for both m¹Ψ and mo⁵U mRNAs, with Pearson correlations of 0.979 and 0.879, respectively (FIG. 5B). These results suggest that the average rate of protein production within the first few hours after RNA delivery is the strong determinant of protein expression for exogenous mRNAs.

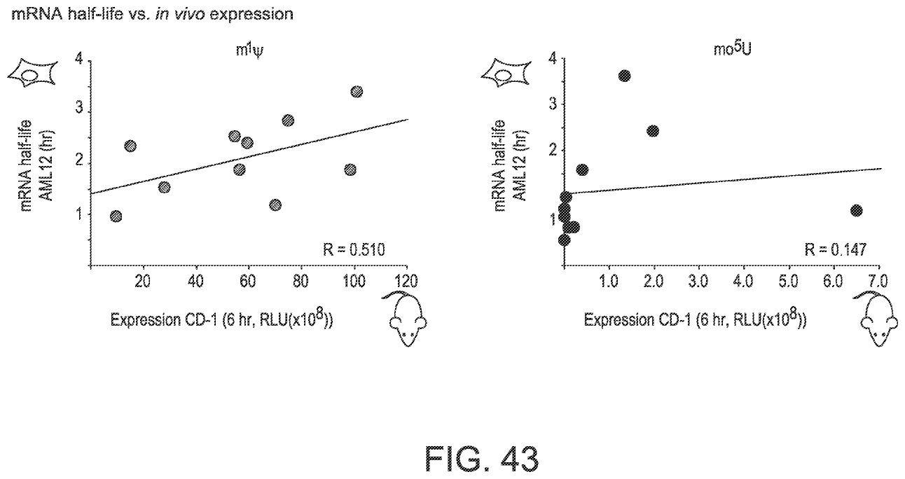

Next, mRNA decay kinetics were examined to determine mRNA half-lives across different sequences and chemistries. Luc mRNAs with m¹Ψ and mo⁵U mRNAs and a negative control mRNA lacking a poly(A) tail were electroporated into AML12 cells and RNA abundance was assayed for the next 32 hours (FIG. 5D). By 7 hours, most of the RNA was degraded and by 24 hours. RNA had returned to background levels (FIG. 5B). Half-lives were calculated for each RNA variant using exponential decay curves. Whereas the tail-less control RNA degraded rapidly (t_{1/2}=30 min), Luc mRNAs half-lives ranged from 0.9 to 3.7 hours for m¹Ψ and 0.5 to 4.1 hours for moU (Table 2 and FIG. 5B). There was a moderate correlation between half-life and expression in vivo (r=0.51) (FIG. 43 and FIG. 5C), in mRNAs containing m¹Ψ, but no such correlation was observed for mRNAs containing mo⁵U (r=0.15) (FIG. 5C). Notably, the range of mRNA half-lives in cells for the m¹Ψ and mo⁵U mRNAs largely overlapped despite their >10-fold range in in vivo protein expression (FIG. 5D). Thus, mRNA stability is unable to account for most of the differences in protein expression between Luc mRNAs with m¹Ψ and mo⁵U.

TABLE 2
Half-lives of Luc mRNAs in AML12 cells

| | m¹Ψ | mo⁵U |
|---|---|---|
| | half-life | half-life |
| mRNA | (hours) | (hours) |
| Tail-less RNA | 0.4844 | 0.5787 |
| (control) | | |
| L₁ | 2.394 | 4.118 |
| L₂ | 2.524 | 2.917 |
| L₇ | 1.874 | 2.075 |
| L₈ | 2.841 | 1.471 |
| L₁₅ | 1.191 | 0.8183 |
| L₁₈ | 3.398 | 1.182 |
| L₂₂ | 2.335 | 1.046 |
| L₂₄ | 0.962 | 0.5303 |
| L₂₉ | 1.878 | 0.8096 |
| L₃₂ | 1.540 | 1.271 |
| Average | 1.947 | 1.624 |

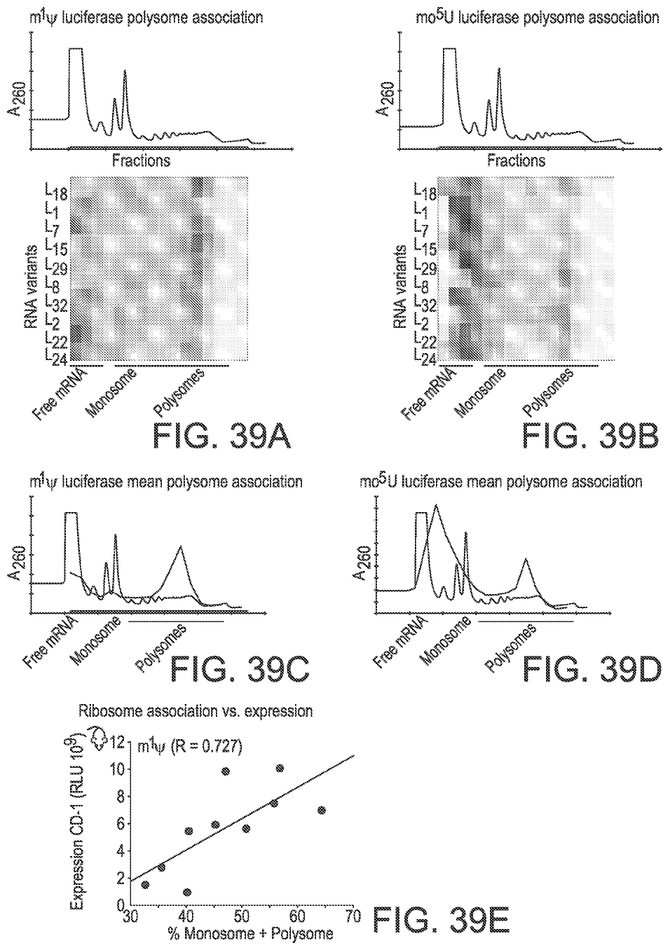

To investigate whether the observed protein expression differences were due to differential engagement of the translation machinery, polysomes profiles were generated. Equimolar pools of ten Luc mRNAs in both m¹Ψ and mo⁵U were transfected into AML12 cells, and 6 hours after transfection, cytoplasmic lysates were fractionated over a sucrose gradient. The relative quantity of each individual mRNA was determined for each gradient fraction using qRT-PCR. Of those mRNAs that were associated with ribosomes, a polysome size of ˜10 was typical across both m¹Ψ and mo⁵U (FIGS. 39A-39B). A trend emerged across different sequence variants with the same modified nucleotide, where polysomes were of similar size across different sequenced variants, but the fraction of mRNAs that associated with ribosomes varied. Within the set of m¹Ψ containing mRNA, highly expressed variants (L₁₈ and L₇) associate with polysomes more than variants that produced less protein, such as L₂₄ (FIG. 39A). A similar trend was observed with the best expressing mo⁵U containing mRNA variant, L₁₈ (FIG. 39B). Averaged over all ten Luc variants, m¹Ψ mRNAs (FIG. 39C) were more frequently associated with ribosomes than were mo⁵U mRNAs (FIG. 39D), with an average of 46.7% of m¹Ψ mRNAs ribosome-associated compared to 31.9% for mo⁵U (p=0.0036, paired Student's t-test). The percent of each m¹Ψ mRNA associated with ribosomal fraction (including monosomes and polysomes) was calculated. These values correlated strongly (R=0.727) with levels of protein expression seen in vivo for the m¹Ψ Luc variants (FIG. 39E), indicating that ribosomal association, particularly in the context of heavy polysomes, largely determines the amount of protein produced by exogenous mRNAs.

### Discussion

mRNA-based therapeutics have gained widespread attention as a novel treatment modality, but a deeper understanding of the principles that dictate their performance is needed. Multiple facets of an mRNA sequence impact protein expression, including codon usage, secondary structure, co-translational protein folding, and many more. This is true for endogenous transcripts (Rodnina, 2016) as well as exogenously delivered mRNAs (Welch et al., 2009). The detailed roles of these factors have been extremely difficult to tease apart because any change to the mRNA sequence affects multiple correlated factors including GC content, codon usage (including codon pairs), and secondary structure. Here, modified nucleotides provide a tool to observe the effects of changes in mRNA secondary structure on protein expression independent of any effects due solely primary sequence changes. It was found that the primary determinants for maximal protein expression are an unstructured region upstream and downstream of the start codon followed by a highly structured ORF.

In the constructs described herein, optimal protein expression was observed when the entire 47 nt 5′ UTR and the first 30 nts of the CDS had minimal structure. The results are consistent with a large body of previous evidence regarding the effects of secondary structure near the start codon. Across all kingdoms of life, regions close to the translation initiation site tend to be relatively free of secondary structure especially in highly expressed genes (Ding et al., 2012; Ding et al., 2014; Gu et al., 2010; Kertesz et al., 2010; Ringner and Krogh, 2005: Robbins-Pianka et al., 2010; Shah et al., 2013; Tuller and Zur, 2015; Wan et al., 2014). Consistent with this, introduction of stable stem loops in the 5′ UTR or encompassing the start codon have been shown to decrease protein expression by interfering with pre-initiation complex scanning (Kozak. 1986) and/or start codon recognition (Kozak, 1989). Further, increasing predicted secondary structure strength toward the 5′ end of a CDS using synonymous substitutions generally decreases protein expression (Allert et al., 2010; Babendure et al., 2006; Goodman et al., 2013; Kudla et al., 2009).

In contrast the 5′ UTR and area around the start codon, the role of secondary structure in the remainder of the CDS is less well studied, with previous data proving somewhat contradictory (Mortimer et al., 2014). On the one hand, transcriptome-wide secondary structure probing data and computational predictions indicate that, when averaged across all transcripts in each species, human, fly, and worm CDSs are slightly less structured than their flanking UTRs (Li et al., 2012. Wan. 2014). This is consistent with data from bacteria indicating a negative correlation between CDS secondary structure and protein output (Li et al., 2012; Supek et al., 2010; Tuller et al., 2010). Secondary structure has been shown, in vitro, to decrease the rate of elongation by increasing ribosome pausing (Chen et al., 2013; Wen et al., 2008). In extreme cases, very large stem-loops in the CDS can trigger No-Go Decay in synthetic constructs (Doma and Parker, 2006; Shoemaker and Green, 2012); such structures, however, are rarely found in natural mRNAs. Thus, it makes intuitive sense that minimizing CDS secondary structure should increase protein output.

Contradicting these findings, however, a small but growing number of studies suggest that CDS secondary structure can be beneficial for functional protein production. In contrast to the examples above, structure probing studies indicate that S. cerevisiae and Arabidopsis CDSs are more structured on average than their flanking UTRs (Kerecz et al., 2010; Li et al., 2012). Additionally, transcriptome-wide comparisons between computational folding and protein expression reveal a positive correlation CDS secondary structure and protein expression in S. cerevisiae (Park et al., 2013 2014; Zur and Tuller. 2012). An early conservation analysis comparing human to mouse mRNAs suggested that wobble positions are under selective pressure to increase basepairing interactions within the CDS, not decrease it as would be expected if CDS secondary structure were solely inhibitory (Shabalina et al., 2006). Finally, recent work has reported a positive correlation between CDS structure and expression of viral, secreted, and membrane proteins (Jungfleish et al. 2017).

The global incorporation of modified nucleotides such as m¹Ψ and mo⁵U was used to modulate CDS secondary structure without altering sequence. By serving to alter secondary structure strength, modified nucleotides thus provide a unique window through which one can specifically interrogate the role of mRNA structure in modulating the efficiency of protein expression without changing the sequence of the mRNA. The present results clearly indicate that increased secondary structure content within the CDS correlate with increased protein expression, at least for the constructs tested here. This increased protein expression from more structured CDSs is not due to increased mRNA half-life (FIG. 43). Also, since the data are based on exogenously delivered mRNA, there is no confounding transcriptional effect that can compromise studies with DNA-based experiments (Newman et al., 2016). It is further demonstrated that, while the primary sequence rules (i.e., codon usage) governing protein expression are non-uniform across modified nucleotides, the positive correlation between high CDS structure and high protein remains constant. The data thus provide a biochemical explanation for the recent finding that m¹Ψ-containing mRNAs produce more protein despite slower translation elongation rates (Svitkin et al., 2017).

Unexpectedly, the polysome profiling data (FIGS. 39A-39E) revealed a relationship between ribosome engagement and CDS structure. That is, protein expression. CDS structure and polysome association are all positively correlated. How increased CDS secondary structure leads to increased ribosome association is an open question. One model suggests that the mRNA structure formed by optimal codons acts to even out translational kinetics governed by tRNA abundance (Gorochowski et al., 2015), thus preventing ribosome traffic jams and permitting optimal elongation rates (Mao et al., 2014). Other mathematical models predict that the optimal ribosome density for productive translation is about one half of the maximum possible density (Zarai et al., 2016). Considering the present findings, it seems reasonable that increased secondary structure within the CDS could help achieve the optimal ribosome density for efficient protein production. Alternatively, regulating the speed of the ribosomes by way of mRNA structure may aid co-translational protein folding, preventing the production of misfolded, inactive protein (Chaney and Clark. 2015). It is also conceivable that a high degree of secondary structure serves to bring the 5′ and 3′ ends of the mRNA into proximity, thereby aiding initiation and reinitiation complex formation (Clote et al., 2012; Yoffe et al., 2011). Finally, mRNAs preferentially associated with the double-stranded RNA-binding protein Staufen1 have both high GC-content (i.e., high CDS structure) and higher ribosome densities than the general population (Ricci et al., 2014).

Determining the biological mechanism(s) determining the correlation between mRNA secondary structure and translational efficiency will require further study. The use of modified nucleotides to manipulate mRNA secondary structure independent of mRNA primary sequence changes has been shown herein to offer a powerful new tool to elucidate basic principles governing protein expression.

### Materials and Methods

mRNA Preparation

Three different proteins, human erythropoietin (hEpo), enhanced green fluorescent protein (eGFP) and firefly luciferase (Luc) were selected and then sequence variants were synthesized in vitro using all unmodified nucleotides or global substitutions of uridine (U) for the modified uridine analogs pseudouridine (Ψ), N¹-methyl-pseudouridine (m¹Ψ), 5-methyoxy-uridine (mo⁵U), or a combination of Ψ and 5-methyl-cytidine (m⁵C) as indicated. These proteins vary in their fundamental properties including biological function, protein structure, amino acid composition, length of coding sequence (from 579 to 1,653 nucleotides), and subcellular localization (intracellular or secreted). In all cases, the coding sequence was flanked by identical 5′ and 3′ untranslated regions (UTRs) capable of supporting high levels of protein expression (FIG. 1B). Thus, total protein expression from these exogenous RNAs is determined by the combined impact of the primary coding sequence and the nucleotides used.

For simplicity and ease of analysis, mRNA sequences based on simple one-to-one codon sets (i.e. each amino acid is encoded by the same codon at every instance of the amino acid that disfavored the use of rare codons) were designed. Regions of increased rare codon frequency have been shown to decrease protein expression and mRNA stability (Presnyak et al., 2015; Weinberg et al., 2016). The hEpo protein contains a 9 amino acid (27 nucleotide) signal peptide sequence that is removed from the mature protein after targeting the protein to the endoplasmic reticulum (ER) for secretion. To evaluate whether codon choice had different effects in the signal peptide region, additional sequence designs were tested for hEpo, in which a leader region of 30 amino acids was encoded using two distinct codon sets: L1 (an AU-rich codon set) and L2 (a GC-rich codon set) (FIG. 1B).

All mRNAs were synthesized by T7 RNA polymerase in vitro transcription reaction (IVT) (New England Biolabs cat. no. M0251L) and purified using standard techniques. All nucleotides in the reaction were applied at final concentration of 100 mM. The following nucleotides were used: all unmodified nucleotides, or unmodified adenosine, cytidine, and guanosine with pseudouridine (Ψ, 1-methyl-pseudouridine (m¹Ψ, or 5-methoxy-uridine (mo⁵U), or unmodified adenosine and guanosine with pseudouridine and 5-methyl-cytidine (Ψ/m⁵C). DNA templates for IVT were generated by PCR amplification of codon-optimized sequences custom-ordered as plasmids from DNA2.0. All mRNAs were capped using the Vaccinia enzyme m⁷G capping system (New England Biolabs M2080S). All mRNA samples were analyzed for purity and cap content by capillary electrophoresis.

### Cell Culture Models

To maintain cells for all in vitro assays, HeLa (ATCC CCL-2), Vero (ATCC CCL-81), BJ (ATCC CRL-2522). HepG2 (ATCC HB-8065) and AML12 (ATCC CRL-2254) cells were maintained in Dulbecco's Modified Eagle's Medium (DMEM) supplemented with GlutaMAX, HEPES, high glucose (Life Technologies, cat. no. 10564-011), 10% fetal bovine serum (FBS) (Life Technologies, cat. no. 10082-147) and sodium pyruvate (Life Technologies, cat. no. 11360-070) at 37° C. in a humidified incubator at 5% CO₂ atmosphere. Cells were passaged every 3-4 days (2 times weekly) with 0.25% trypsin-EDTA solution (Life Technologies, cat. no. 25200-056) and washed with sterile PBS (Life Technologies, cat. no. 10010-049) under sterile aseptic conditions, for no more than 20 passages.

For all in vitro assays carried out in primary human hepatocytes, cryopreserved primary human hepatocytes (ThermoFisher cat. no. HMCPIS) were thawed and plated for use in CHRM (ThermoFisher cat. no. CM7000). Williams Medium E supplemented with Hepatocyte Plating Supplement Pack (Serum-Containing), immediately. Plates were incubated at 37° C. in a humidified incubator at 5% CO₂ atmosphere 5 hours before changing media to serum free media (William's E Maintenance Media—Without Serum). Plates were incubated at 37° C. in a humidified incubator at 5% CO₂ atmosphere for all periods between active uses.

mRNA Transfection

To measure protein expression and/or innate immune induction for all in vitro assays, HeLa, Vero, BJ, AML12 and primary hepatocytes were seeded in 100 uL per well of 96 well plate at a concentration of 2×10⁵ cells/mL one day prior to transfection and incubated overnight under standard cell culture conditions. For transfection, 50 ng of mRNA was lipoplexed with 0.5 uL Lipofectamine-2000 (ThermoFisher cat. no 11668027), brought to a volume of 20 uL with a quantity sufficient of Opti-MEM (ThermoFisher cat. no. 31985062) and directly added to cell media. All transfections were performed in duplicate.

### Expression Assays

To detect level of protein expression for all transfected Luciferase exogenous mRNA, single endpoint Luciferase expression assays were conducted 24 hours post transfection, unless otherwise specified. The Luciferase Assay System (Promega cat. no. E1501) was used as per manufacturer's suggested protocol with 100 uL lysis buffer at 1:10 dilution with Luciferase assay reagent and luminescence was measured on the Synergy H1 plate reader.

To detect level of protein expression for all transfected hEPO exogenous mRNA, single endpoint hEPO expression assays were conducted 24 hours post transfection, unless otherwise specified. The Human Erythropoietin Platinum ELISA kit (Affymetrix cat. no. BMS2035) was used as per manufacturer's suggested protocol.

To detect level of protein expression for all transfected cGFP exogenous mRNA, single endpoint eGFP expression assays were conducted 24 hours post transfection, unless otherwise specified. Cells were analyzed for fluorescence at an excitation wavelength of 488 nm and emission wavelength of 509 nm on the Synergy H1 plate reader.

To detect level of innate immune induction in BJ resulting from introduction of exogenous mRNA, single endpoint interferon-beta (IFN-β) expression assays were conducted on cell supernatant 48 hours post transfection. The human IFN-Beta ELISA kit (R&D Systems cat. no. 41410) was used as per manufacturer's suggested protocol.

### In Vivo Studies

To confirm expression levels observed in vitro translate to more advanced biological systems, reporter protein expression (hEpo and Luc) from exogenous mRNA in CD-1 and BALB/C mouse models was measured.

To detect level of protein expression for all Luciferase exogenous mRNAs, all mRNAs were formulated in MC3 lipid nanoparticles at a concentration of 0.03 mg/mL, administered intravenously to CD-1 mice at a dose of 0.15 mg/kg of body mass and measured for expression by whole body Bioluminescence Imaging (BLI) at 6 hours post injection.

To detect level of protein expression for all hEPO exogenous mRNAs, all mRNAs were formulated in MC3 lipid nanoparticles at a concentration of 0.01 mg/mL, administered intravenously to BALB/C mice at a dose of 0.05 mg/kg of body weight, and measured for serum hEPO concentration using Human Erythropoietin Quantikine IVD ELISA kit (R&D Systems cat. no. DEP00) at specified times (6 hours) post-injection.

### UV Melting

As RNA is heated, the normalized first derivative of UV absorbance is a measure of the amount of RNA structure that melts at a given temperature. To assess the global secondary structure of select mRNAs that displayed different levels of protein expression, UV absorbance was measured through multiple heat-cool cycles. Absorbance was measured at 260 nm on the Cary100 UV Vis Spectrometer as RNA, in 2 mM sodium citrate buffer (pH=6.5) was heated from 25° C. to 80° C. at a rate of 1° C./minute, and then cooled from 80° C. to 25° C. at a rate of 1° C./minute. This cycle was repeated three times in total. First derivative of absorbance values were then analyzed as a function of temperature to inform on changes in global secondary structure across changes in primary coding sequence and/or nucleotide chemistry.

### Determination of Nearest-neighbor Thermodynamic Parameters

To refine and extend analysis of the thermodynamics of RNA folding as a function of nucleotide chemistry. UV-melting experiments were performed on 39 synthetic RNA duplexes with Ψ, m¹Ψ, and mo⁵U instead of uridine. Synthetic duplexes were designed such that resulting data could subsequently be processed to obtain the full thermodynamic parameters for the nearest neighbor free energy contributions for each U-derivative tested using established methods (Xia et al., 1998).

Raw data for the determination of the modified RNA duplex nearest-neighbors was collected through absorbance versus duplex melting temperature profiles over six different synthetic oligonucleotide concentrations in 1M NaCl, 10 mM Na₂HPO₄, and 0.5 mM Na₂EDTA, pH 6.98 salt buffer. These data were then processed using Meltwin v.3.5 to obtain a full thermodynamic parameter set through two different methods, those methods being the LnCt/4 vs. Tm⁻¹ method and the Marquardt non-linear curve fit method.

### SHAPE-MaP

To investigate the impact of both primary coding sequence and nucleotide chemistry modification on the biophysical conformation of the mRNA, selective hydroxyl-acylation analyzed by primer extension and mutational profiling were used.

All purified IVT RNAs were denatured at 80° C. for 3 minutes prior to analysis. After denaturation, RNAs were folded in 100 mM HEPES. pH 8.0, 100 mM NaCl and 10 mM MgCl₂ for 15 minutes at 37° C. All RNAs were then selectively-modified with 10 mM 1-methyl-6-nitroisatoic anhydride (1M6) for 5 minutes at 37° C. Background (no SHAPE reagent) and denatured (SHAPE modified fully denatured RNA) controls were prepared in parallel.

After SHAPE modification. RNA was purified and fragmented using 15 mM MgCl₂ at 94° C. for 4 minutes. Purified fragments were then randomly primed with N₉ primer at 70° C. for 5 minutes. Primer extension was carried out in 50 mM Tris-HCl, pH 8.0, 75 mM KCl, 1 mM dNTPs, 5 mM DTT and 6.25 mM MnCl₂ with Superscript II reverse transcriptase (ThermoFisher cat. no. 10864014) for 3 hours at 45° C. The remaining RNA-seq library prep protocol was done with the NEBNext Ultra Directional RNA Library Prep Kit for Illumina (New England Biolabs cat. no. E7420) according to the manufacturer's standard protocol.

RNAseq libraries were sequenced on the Illumina MiSeq using 50 cycle sequencing kit. Ensuing raw sequencing data were analyzed using the publically available ShapeMapper software (Siegfried et al., 2014). Resulting reactivity data were analyzed using a sliding window (median SHAPE) approach to quantify the degree of structure at each position in the RNA as has previously been described (Watts et al., 2009).

### Polysome Profiling

Polysome profiling was used to determine changes in polysome association as a function of coding sequence and/or nucleotide chemistry modification. HepG2 and AML12 cells were pelleted and lysed 6 hours post-transfection. Lysed cells were again centrifuged to remove cell debris. Supernatants were then run on a 20%-55% sucrose gradient using the Gradient Master system and separated into 16 or 30 fractions. Absorbance at 254 nm was monitored to ensure fraction numbers represented increased ribosomal densities.

Fluorescent dye-labeled probes complementary to the Luc variants of interest were synthesized. To determine CT values for each variant in each fraction, qPCR was performed using the TaqMan RNA-to-CT 1-Step kit (ThermoFisher cat. no. 4392938) as per manufacturer's suggested protocol. As each fraction was tied to a ribosomal density. CT values across fractions were then analyzed to determine mean number of ribosomes associated with a variant as well as the percent of transcripts associated with polysomes.

### Ribosome Footprinting

Ribosome footprinting was used to determine changes in ribosome association as a function of coding sequence and/or nucleotide chemistry modification. HepG2 and AML12 cells were lysed post-transfection and centrifuged to remove cell debris. Supernatants were isolated then subjected to nuclease digestion with RNase T1, RNase A and RNAse I (Ambion cat. no. AM2294) at 22° C. for 1 hour. A 20-55% polysome gradient was run as previously described and monosome fraction was isolated. RNA from monosome fraction was then isolated by a phenol:chloroform extraction and treated with polynucleotide kinase (New England Biolabs cat. no. M0201).

Ribosome footprints were size-selected and purified on TBE/urea gel stained with SybrGold for 10 minutes. Upon UV illumination, gel slice from 20-34 nt was selectively enriched and placed in 400 mM NaOAc, pH 5.2. After extraction and isolation. RNA was precipitated in ethanol overnight at −20° C. overnight.

cDNA was generated using the SMARTER (Clontech cat. no. 634925) as per the manufacturer's suggested protocol, rRNA was depleted as previously described (Ignolia, 2012). Remaining RNA-seq library prep protocol was done with the NEBNext Ultra Directional RNA Library Prep Kit for Illumina (New England Biolabs cat. no. E7420) according to the manufacturer's standard protocol. RNAseq libraries were sequenced on the Illumina MiSeq using 50 cycle sequencing kit.

### Quantification and Statistical Analysis

### Comparison of Codon Effects on Translation

Luc expression values from 39 Luc variants were used in 865 pairwise comparisons between synonymous codons to yield p-value testing whether inclusion of specific codons impacted protein expression by ANOVA. Graph Pad software was used to determine p-values and p-values <0.05 were considered significant.

### Determination of Structure Function Relationship in SHAPE Data

The sliding window average of SHAPE reactivates from every position within the RNA were compared to the expression levels determined in HeLa cells. Linear regression was used to determine the degree of correlation between SHAPE and protein

### Example 13: Defining the Structure-Function Relationships within Modified RNAs

### Relationship Between RNA Structure and Function

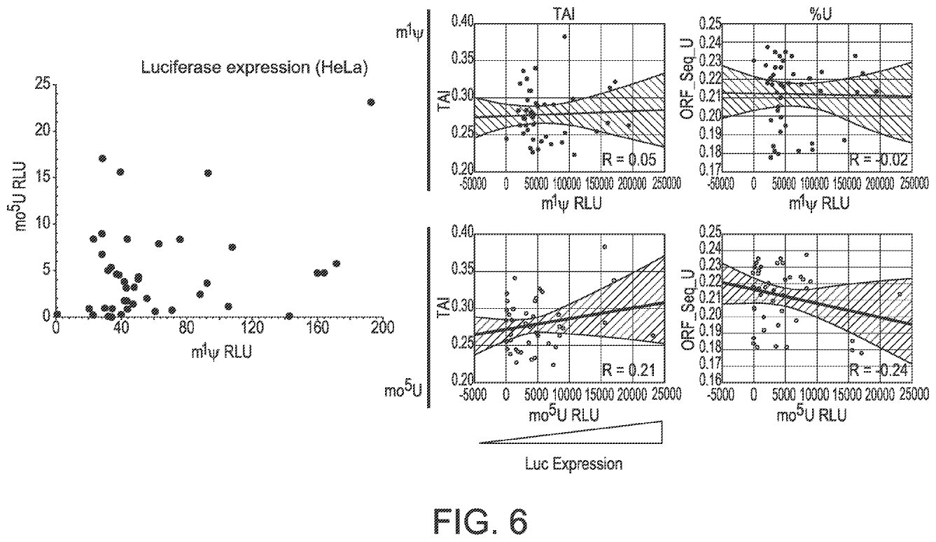

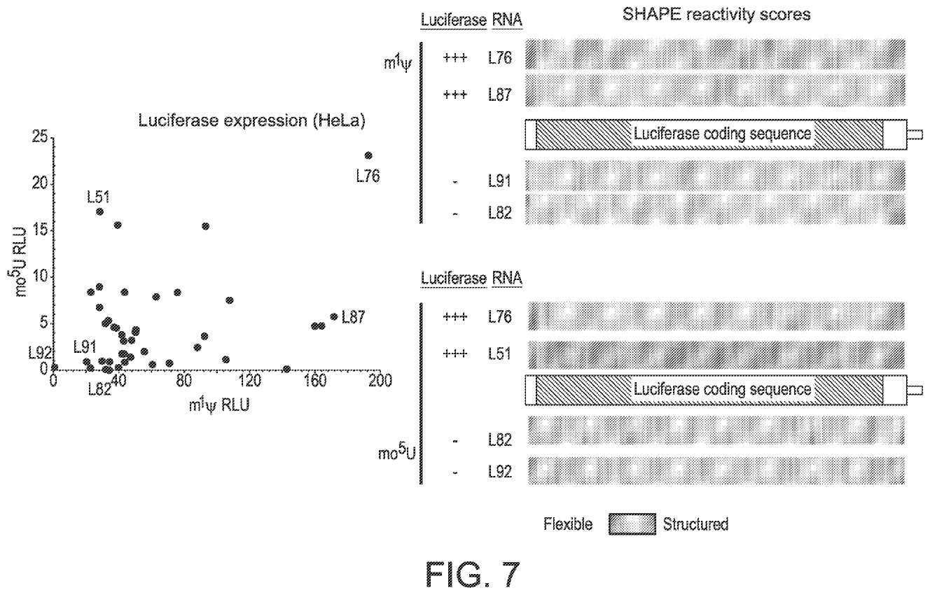

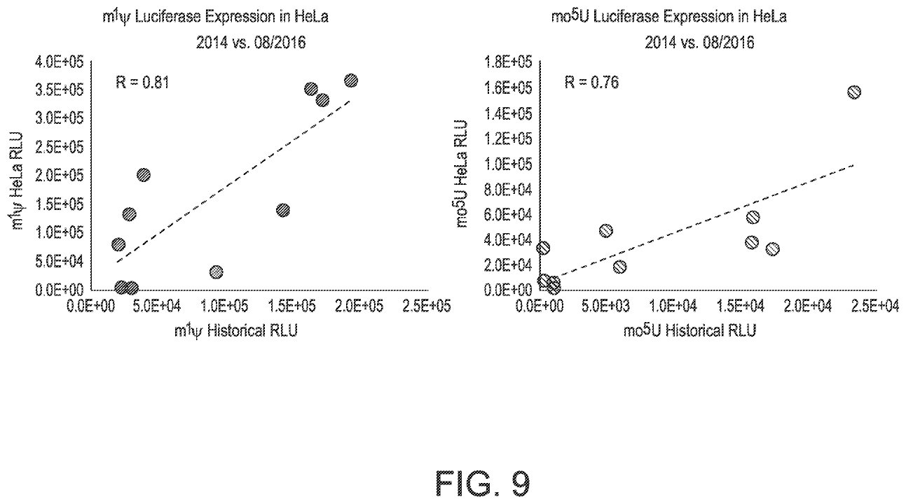

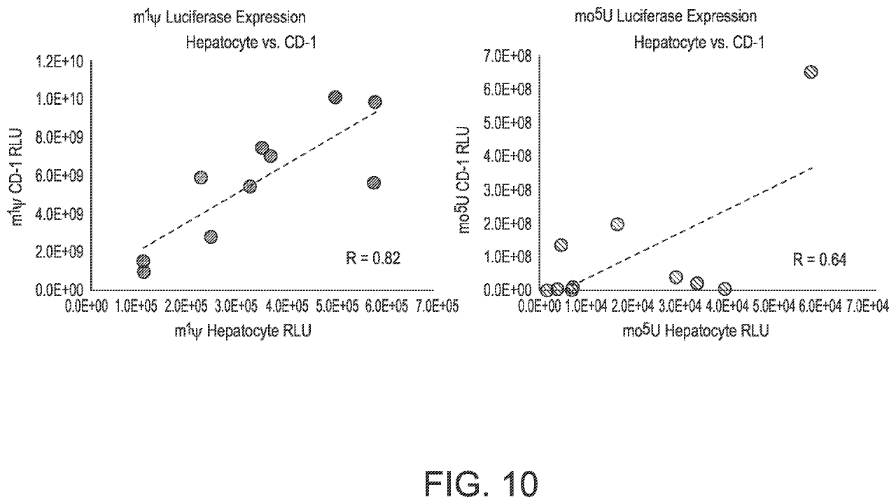

Traditional metrics of primary sequence are poor predictors of chemistry-specific expression, as shown in FIG. 6, whereas biochemical data reveal structure-function relationships (FIG. 7). FIG. 7 depicts SHAPE reactivity scores, showing the different between different luciferase variants (L76, L87, L91, and L82) and the effect of different chemistries (m¹Ψ and mo⁵U). Structure-function relationships have been found to be dependent on position within the RNA (FIG. 8). Flexibility in the 5′ region leads to higher expression, as does structure in the open reading frame (ORF). The expression patter of luciferase sequences were confirmed across production batches and processes (FIG. 9) and in vitro expression assays were found to be moderately predictive of expression in vivo. The in vivo study included the intravenous administration of 0.15 mg/kg of luciferase mRNA in MC4, and then assayed for expression at 6 and 24 hours (FIG. 10).

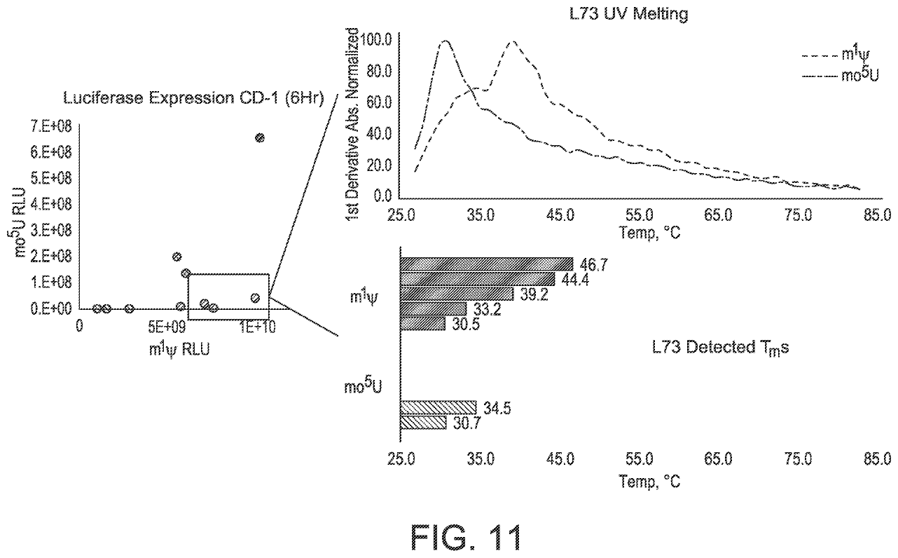

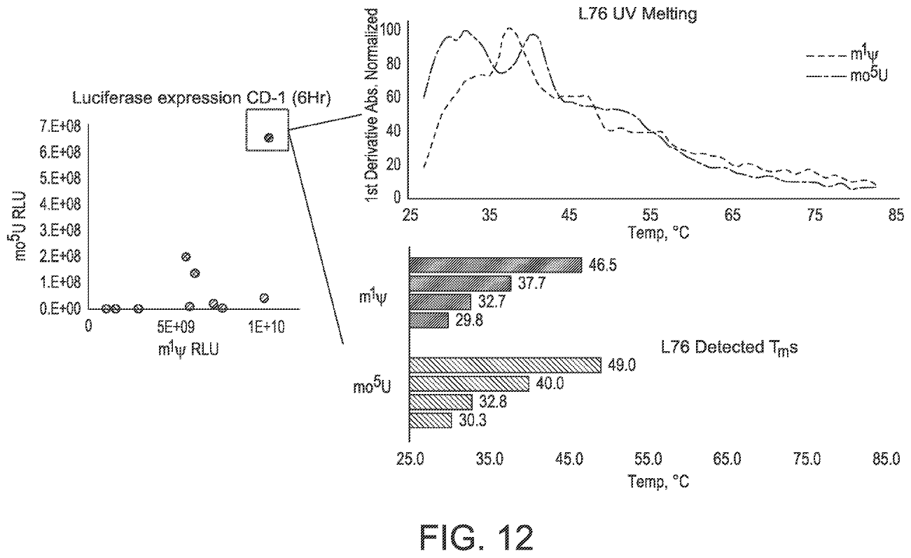

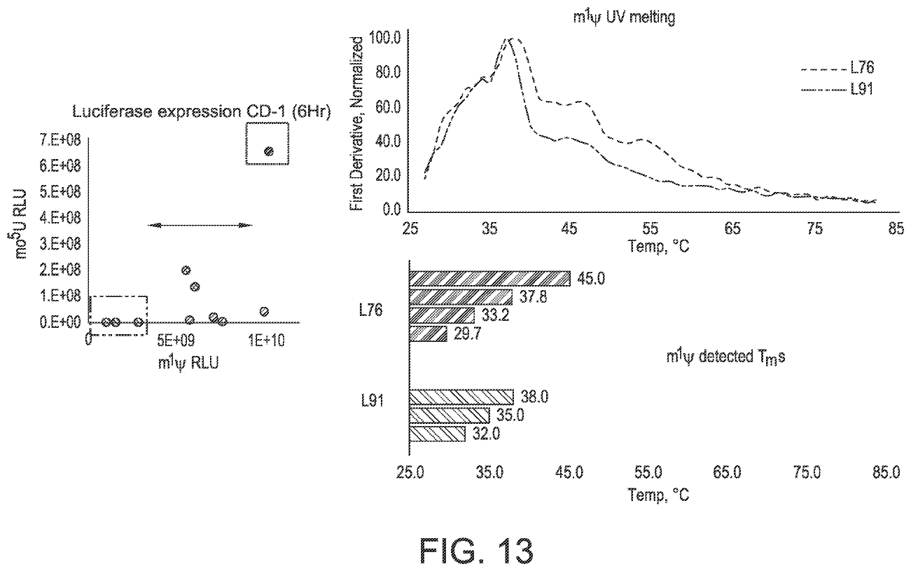

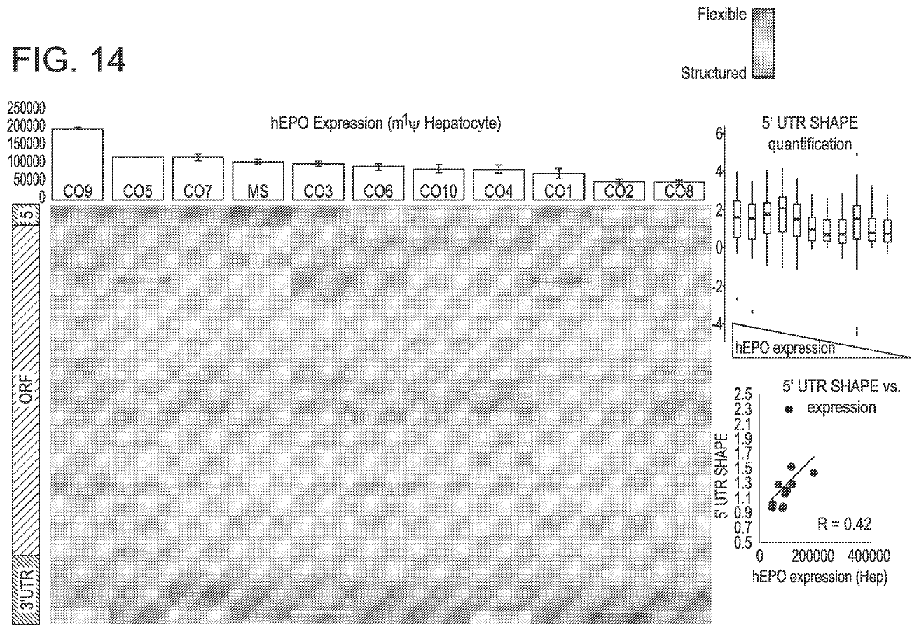

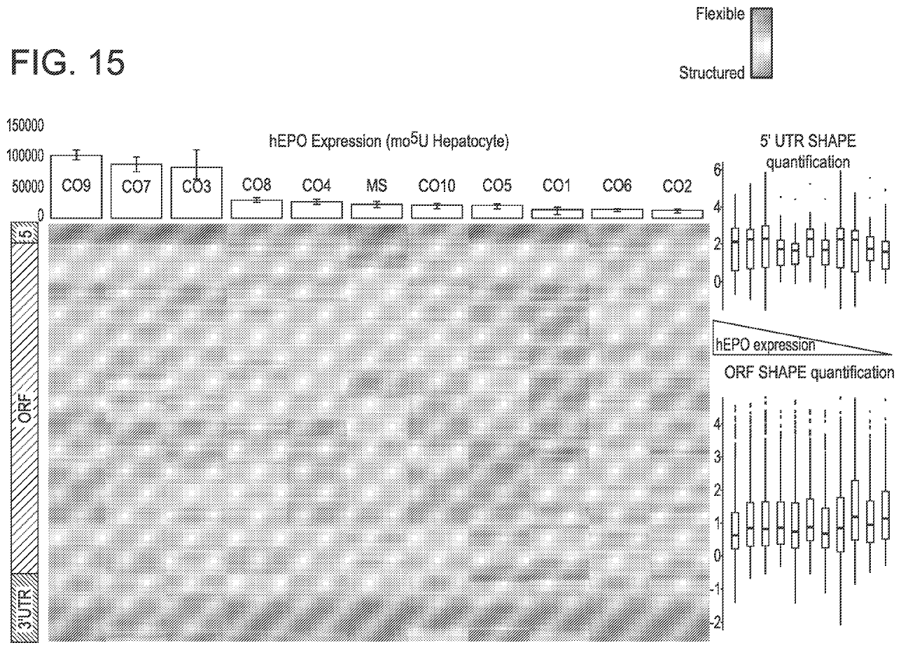

Sequences that display chemistry-dependent expression showed different UV melting profiles (FIG. 11); high-expressing mo⁵U sequences adopted a physical profile that was more similar to the m¹Ψ sequences (FIG. 12). Further, it was found that high- and low-expressing sequences of uniform chemistry can be differentiated by their melting profiles (FIG. 13). Structure-function relationships were found to be consistent across reporter proteins (FIGS. 14 and 15).

### Validation of Sequence-Dependent RNA Functional Expression

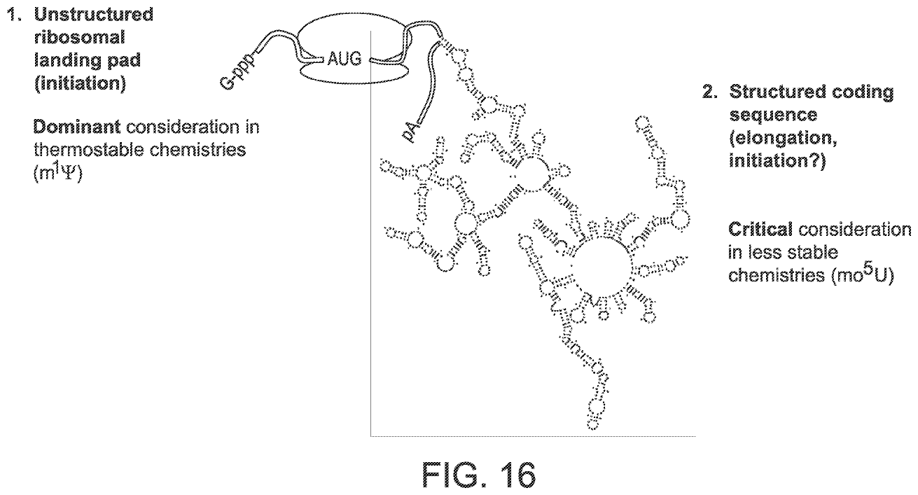

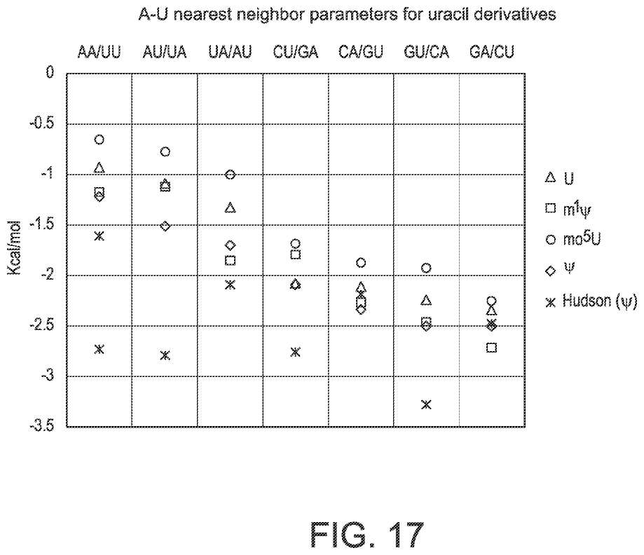

It was found that RNA structure is the product of its primary sequence and its nucleotide chemistry. In one thermostable chemistry (m¹Ψ), it was found that the “thumb” section of the RNA (the unstructured ribosomal landing pad and the site of initiation) was the dominant consideration, whereas in less stable chemistries (mo⁵U), the second section, as a structured coding sequence, was more important (FIG. 16).

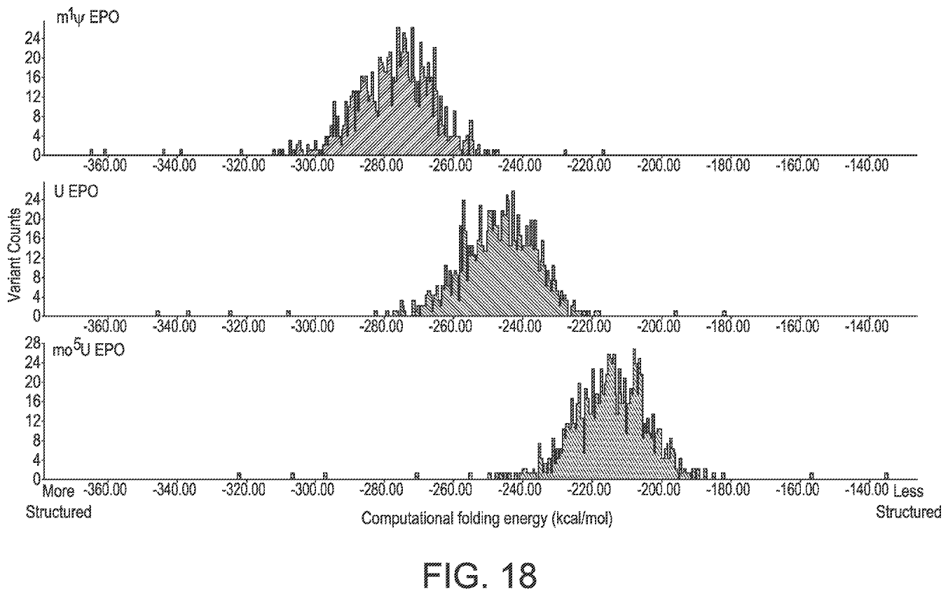

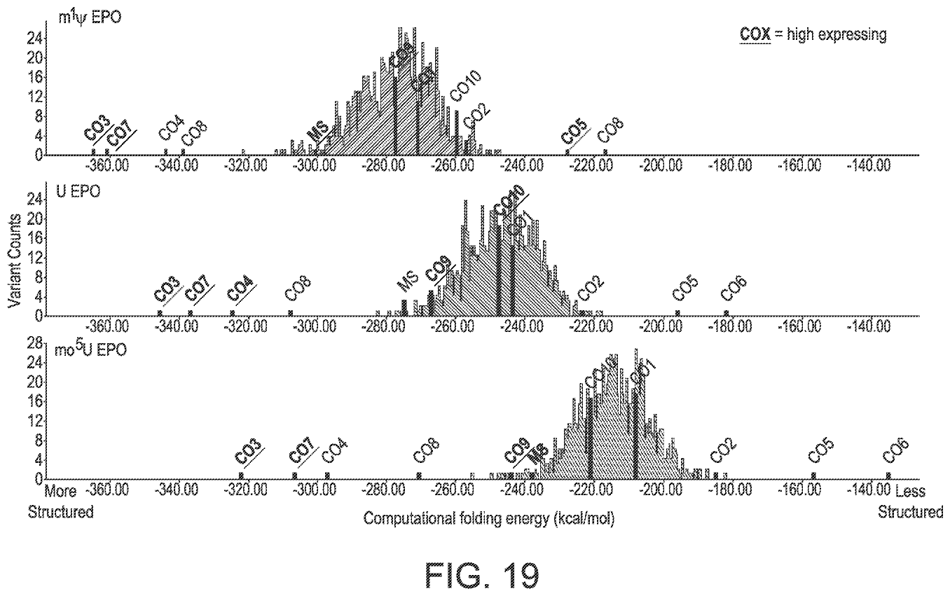

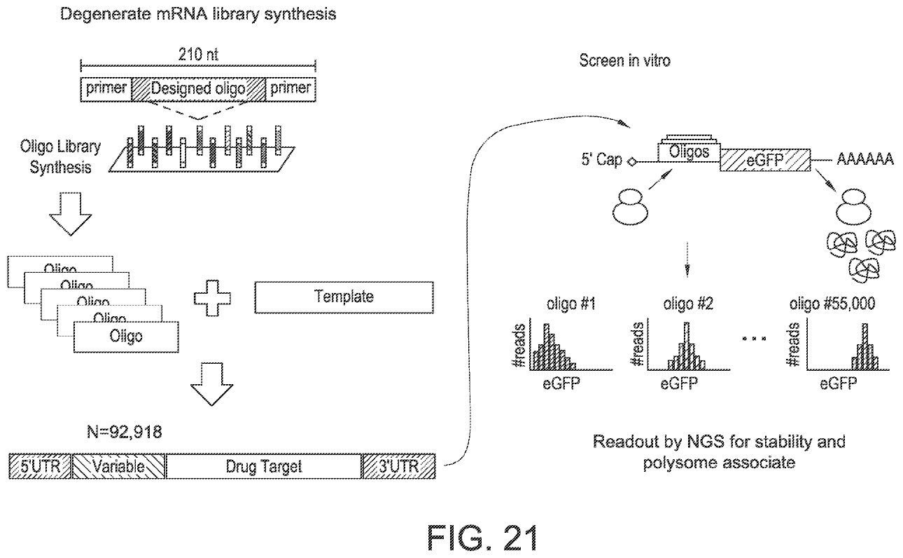

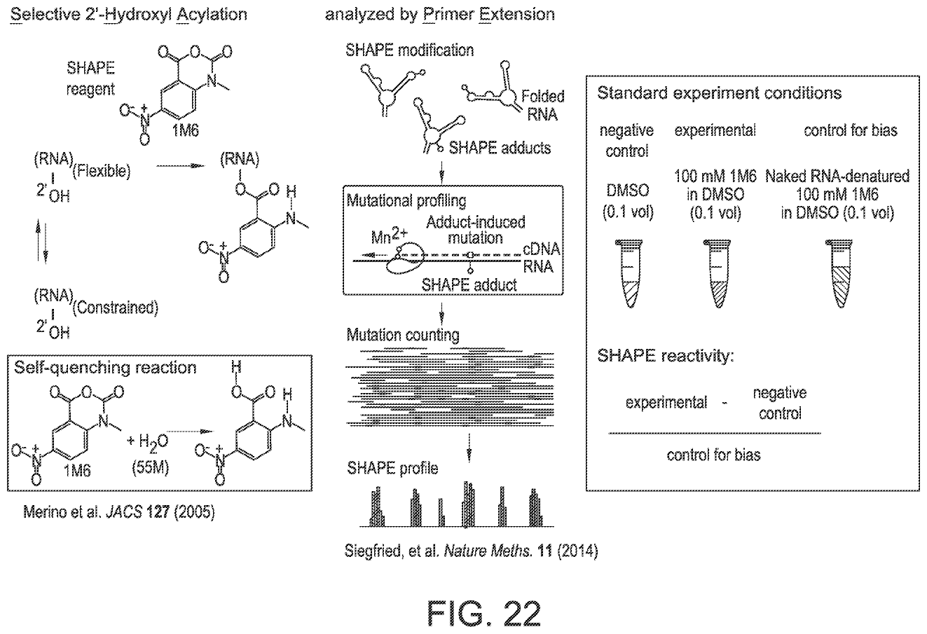

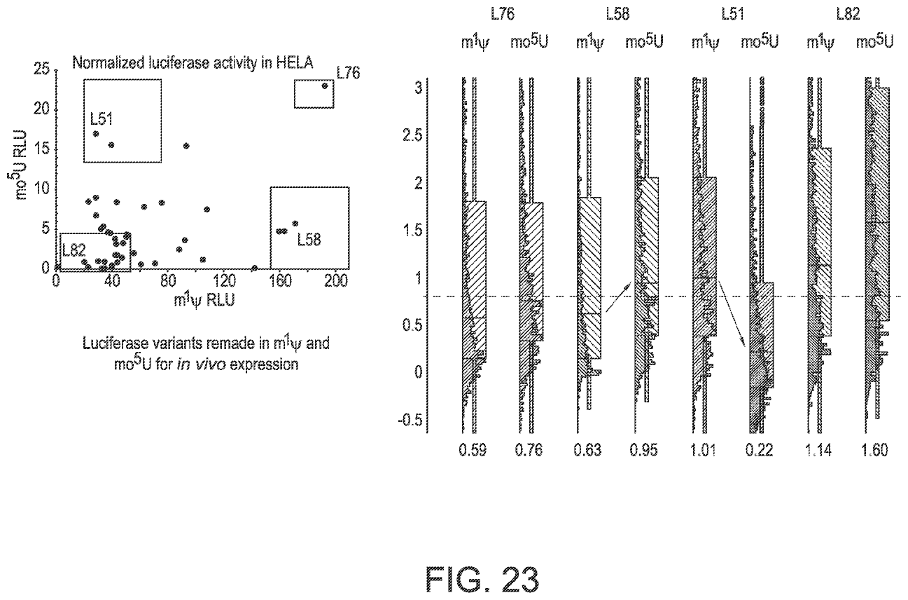

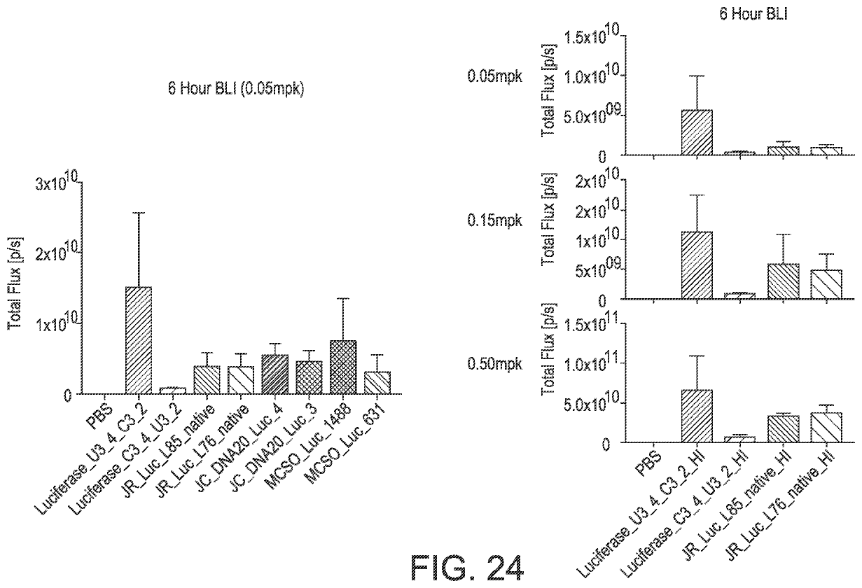

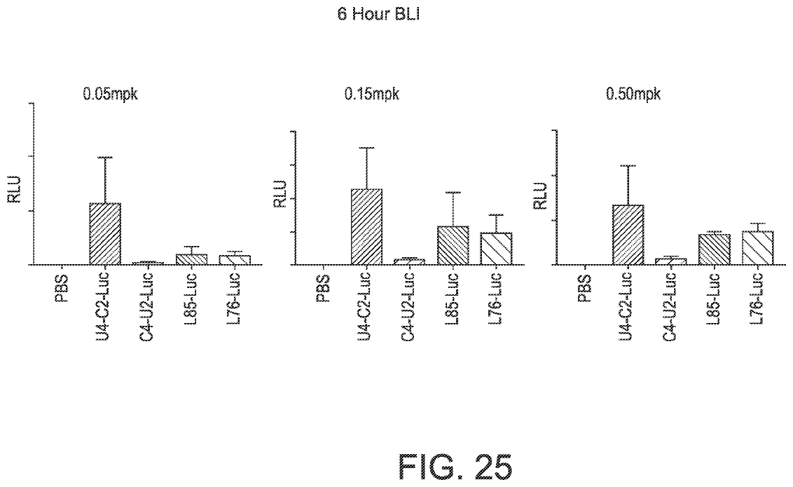

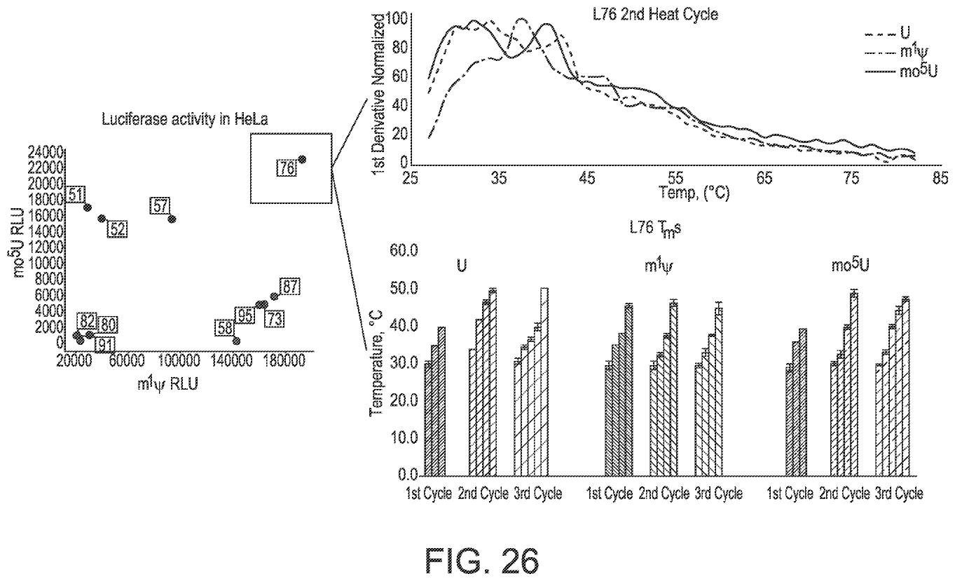

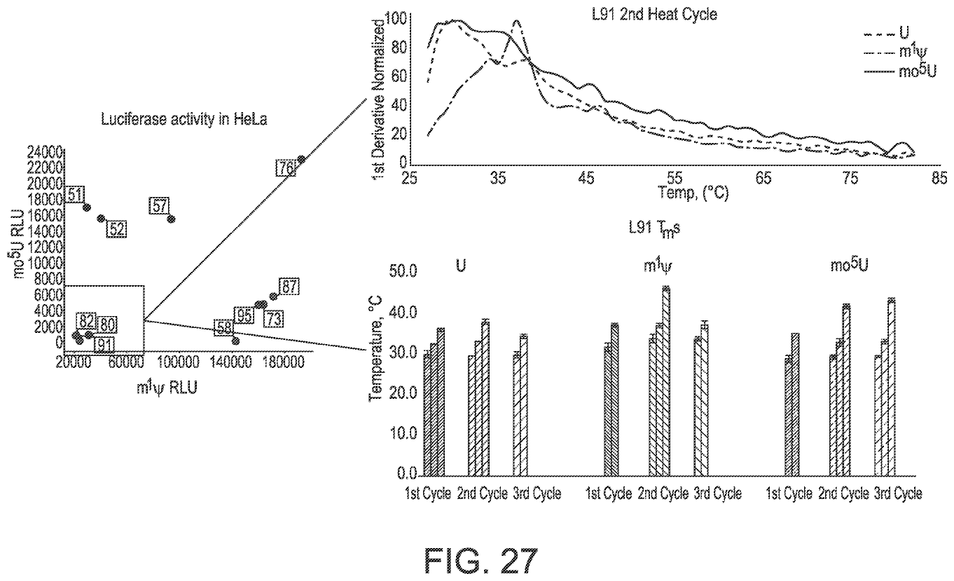

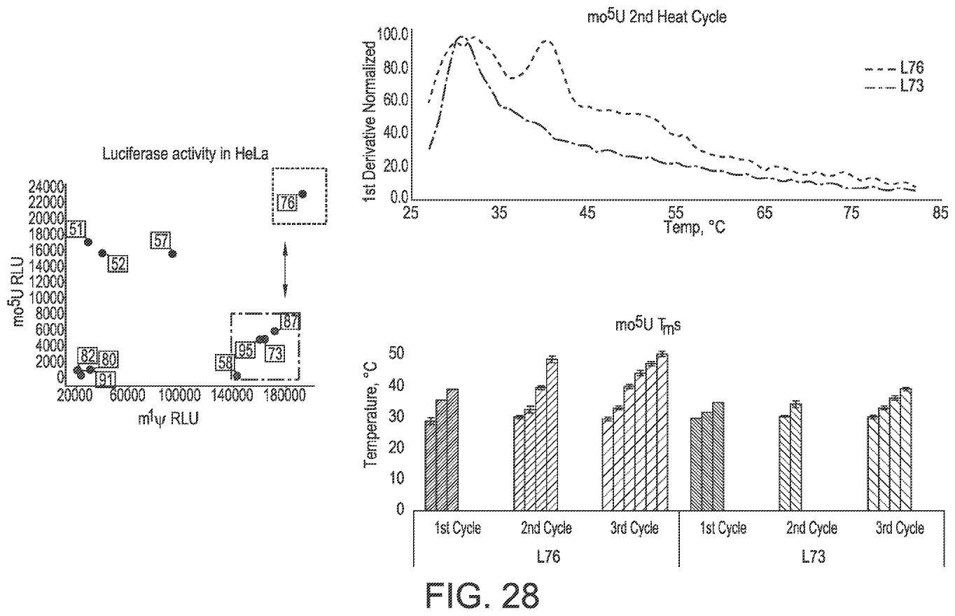

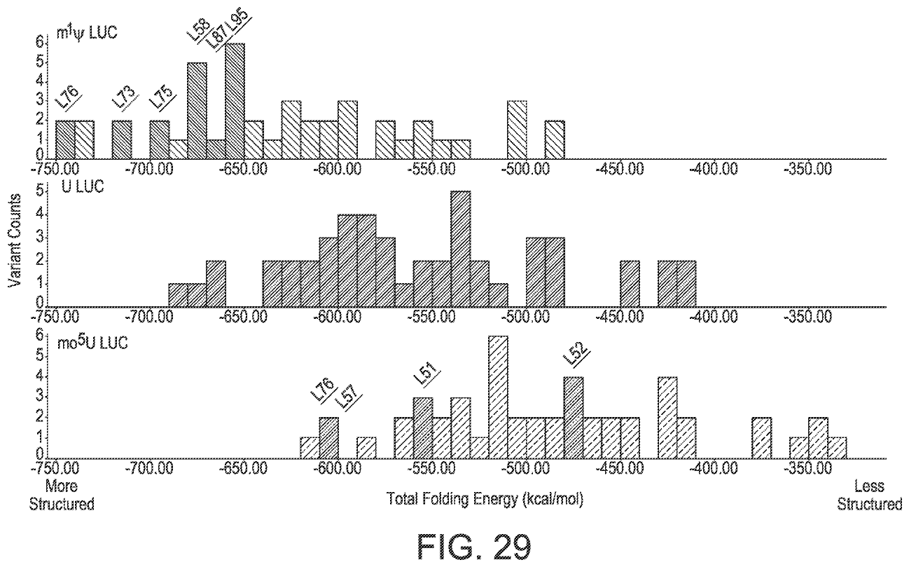

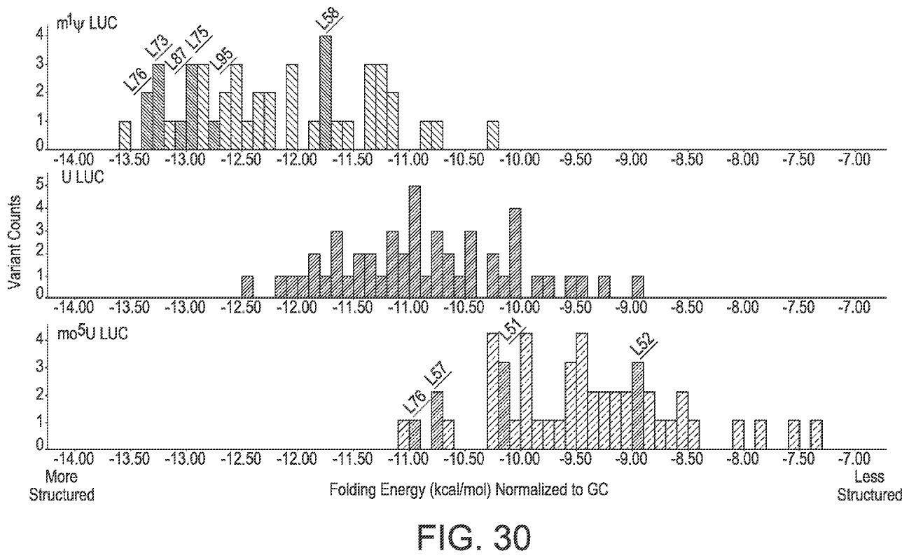

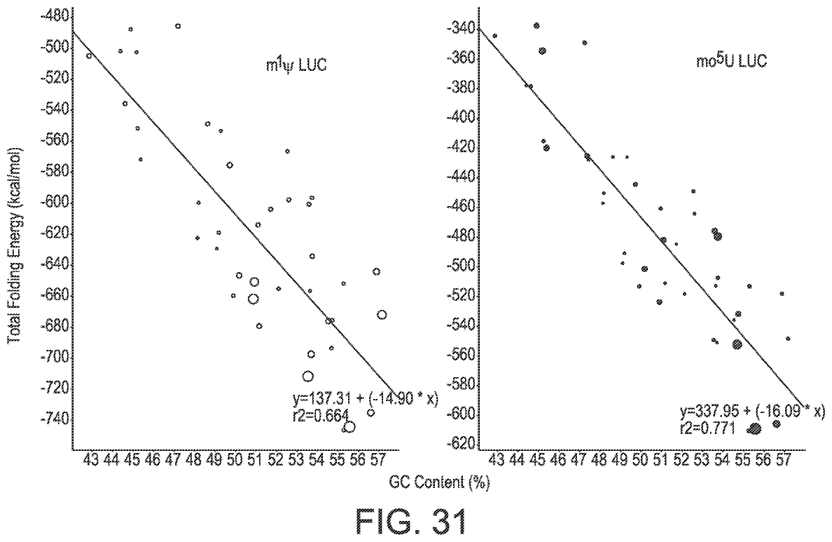

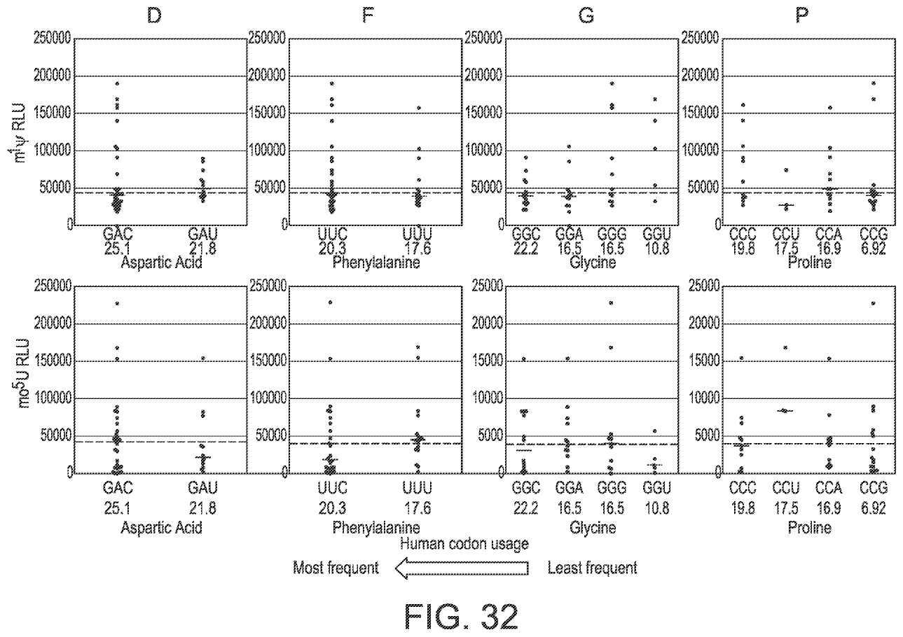

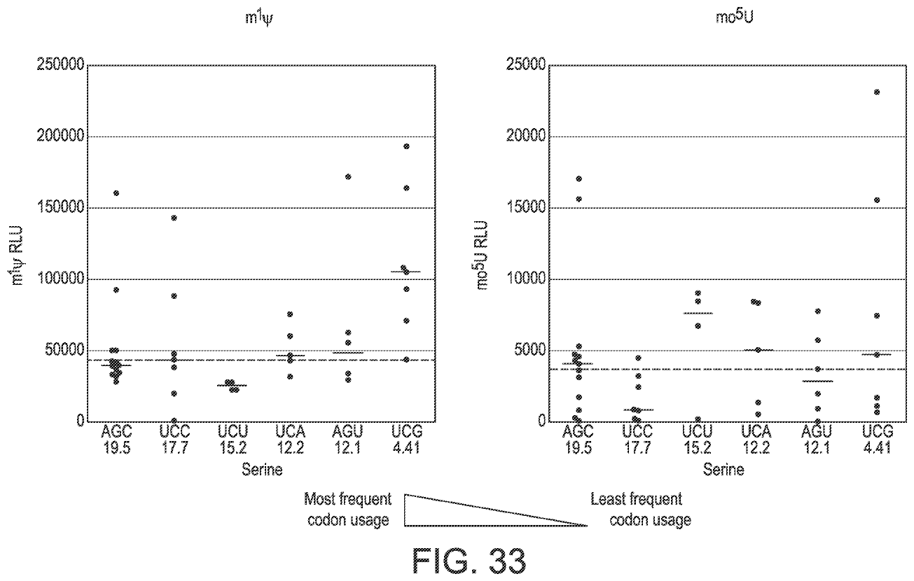

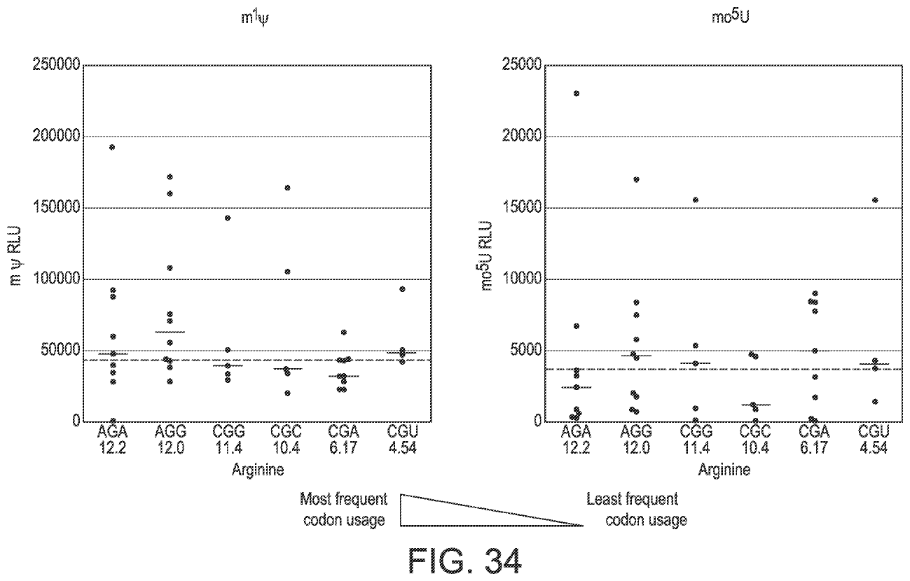

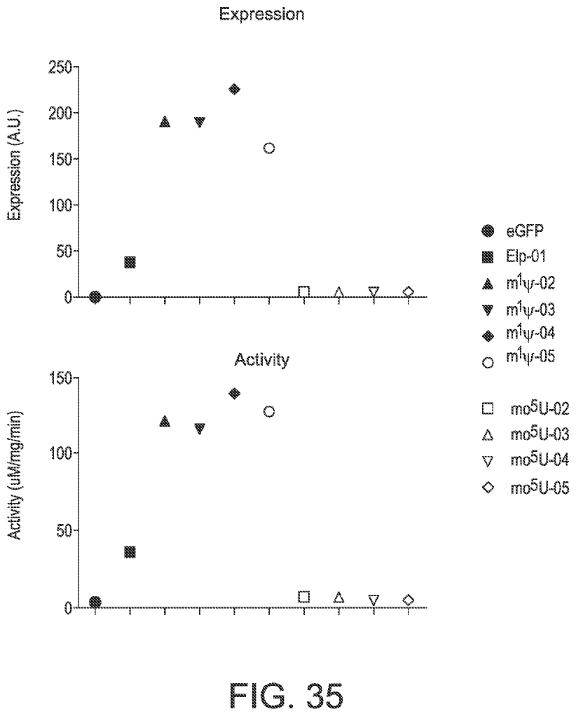

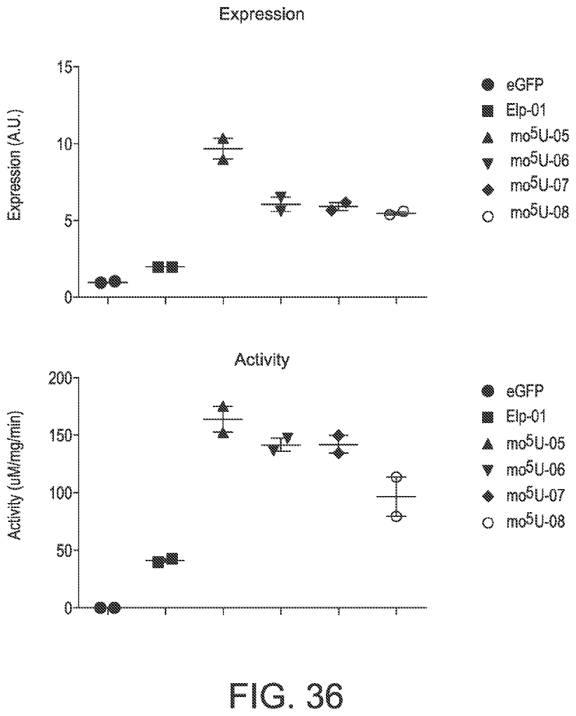

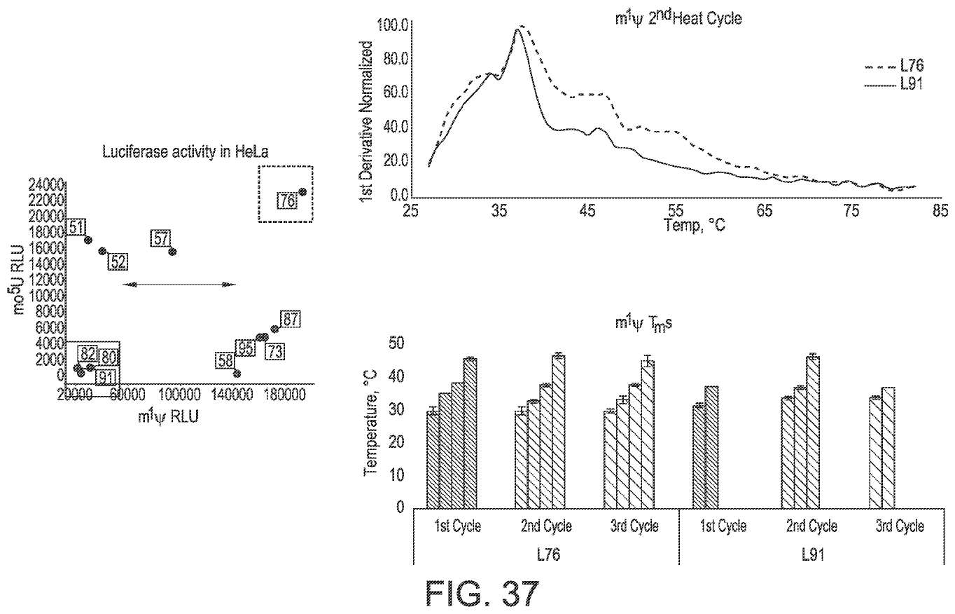

It was found that, with random hEPO sequences, the distribution of minimum free energy (MFE) shifts as a function of nucleotide chemistry (FIG. 18). The propensity for generating high expressing mRNA can then be explained by the distribution shift (FIG. 19); the lower the MFE, the more structured the mRNA, and the greater the resulting protein expression. The hypothesis was validated through a series of experiments rescuing expression (FIG. 20) and massively-parallel screening of ORF variants (FIG. 21).

The structure of mRNAs was found to contribute to potency and can account for much of the observed chemistry-specific differences in expression. Biochemical studies suggest a model for sequence engineering, wherein mRNA is split into two regions: a relatively unstructured “thumb” region followed by a structured ORF (coding) region. Chemistry-specific structure prediction enables tailored sequence engineering approaches, while NGS-based library screening approaches of thousands of sequence variants will enable further refinements to structure-driven sequence engineering.

### Metrics to Predict Increased Protein Expression

mRNA can be structurally engineered to express higher levels of a given protein. The design consists of two regions. In the first region, containing the 5′ UTR and the first 10 codons of the open reading frame, there is a computational prediction average pairing probability across of the region less than 30% and a SHAPE reactivity score of over 1.5, meaning the region is flexible and relatively unstructured. The second region, containing the remaining ORF and the 3′ UTR, has a relatively stable secondary RNA structure, as greater than 50% of the secondary structure is formed at 37° C. as defined by UV-melting analysis, its minimum free energy is within the top 0.1% as defined computationally of synonymous variants, and the median SHAPE reactivity score of the population is less than 0.8.

As described above, in sequences with m¹Ψ or mo⁵U chemistry, high expressing sequences were found to be more thermostable than their low expressing counterparts. It was found that the sequence variants were sensitive to different nucleotide chemistries, as there is a propensity for generating high expressing mRNA sequences can be explained by a distribution shift (FIG. 18). Further, high-expressing luciferase variants were found to have low MFE, independent of GC content.

### EQUIVALENTS

Those skilled in the art will recognize, or be able to ascertain using no more than routine experimentation, many equivalents to the specific embodiments of the disclosure described herein. Such equivalents are intended to be encompassed by the following claims.

All references, including patent documents, disclosed herein are incorporated by reference in their entirety.

1-25. (canceled)

26. A method for producing highly expressing mRNA, comprising:
(a) determining a flexibility value for each nucleotide within a population of synonymous RNA;
(b) determining a SHAPE reactivity for each RNA corresponding to the primary sequence and chemistry of the nucleotides based on the combined flexibility values of the nucleotides;
(c) selecting a RNA from the population having a SHAPE reactivity of less than 1.0; and
(d) synthesizing highly expressing mRNA based on the primary sequence and chemistry of the nucleotides of the selected RNA having a SHAPE reactivity of less than 1.0.

27. The method of claim 26, wherein the highly expressing mRNA is determined to be highly expressing relative to a corresponding wild type chemically unmodified RNA and the highly expressing mRNA produces more protein than the wild type RNA.

28. The method of claim 27, wherein the highly expressing mRNA produces at least 10% more protein than the wild type RNA.

29. The method of claim 26, wherein the highly expressing mRNA has a SHAPE reactivity of less than 0.8.

30. The method of claim 26, wherein the primary sequence of the RNA has a low U content, wherein less than 24% of the nucleotides are U.

31. The method of claim 26, wherein the primary sequence of the RNA is thermodynamically stable.

32. The method of claim 31, wherein at least some of the nucleotides have a 5-methoxy-uridine chemical modification.

33. The method of claim 26, wherein the primary sequence of the RNA is thermodynamically unstable.

34. The method of claim 33, wherein at least some of the nucleotides have a N1-methyl-pseudouridine or pseudouridine chemical modification.

35. The method of claim 26, wherein the highly expressing mRNA has a mRNA minimum free energy (MFE) value within a top 0.1% of low MFE as defined computationally of synonymous variants.

36. The method of claim 26, wherein the highly expressing mRNA has secondary structure capability and wherein greater than 50% of the mRNA forms secondary structure at 37° C. as defined by UV-melting analysis.

37. The method of claim 26, wherein the highly expressing mRNA has secondary structure capability and wherein greater than 70% of the thermostable mRNA forms secondary structure at 37° C. as defined by UV-melting analysis.

38. The method of claim 26, wherein the highly expressing mRNA has secondary structure capability and wherein greater than 90% of the thermostable mRNA forms secondary structure at 37° C. as defined by UV-melting analysis.

39-61. (canceled)

62. A method of synthesizing a thermostable mRNA, comprising:
(a) binding a first polynucleotide comprising a flexible region comprising a first set of nucleotides having a primary sequence and including a 5′ untranslated region (UTR), wherein the first set of nucleotides encoding the 5′ UTR have a first flexibility value based on folding conformation propensity of the primary sequence and thermodynamic stability of nucleotide chemistry, wherein the first polynucleotide is conjugated to a solid support, and a second polynucleotide comprising a thermostable region comprising a second set of nucleotides having a primary sequence and including at least a portion of an open reading frame (ORF), wherein the second set of nucleotides encoding the ORF have a second flexibility value;
(b) ligating the 3′-terminus of the first polynucleotide to the 5′-terminus of the second polynucleotide under suitable conditions, wherein the suitable conditions comprise a DNA Ligase, thereby producing a first ligation product;
(c) ligating the 5′ terminus of a third polynucleotide comprising a 3′-UTR to the 3′-terminus of the first ligation product under suitable conditions, wherein the suitable conditions comprise an RNA Ligase, thereby producing a second ligation product; and
(d) releasing the second ligation product from the solid support,
thereby producing the thermostable mRNA.

63-64. (canceled)
